SEQUENCE LISTING (44 sequences)

SEQ ID NO: 1 - protein, 287 aa, Homo sapiens
  REGION 1..287: note = misc_feature - UniProtKB - Q6GTX8 (LAIR1_HUMAN), Human LAIR-1a (isoform 1)
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 2 - protein, 144 aa, Homo sapiens
  REGION 1..144: note = misc_feature - human LAIR-1
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLY

SEQ ID NO: 3 - protein, 263 aa, Mus musculus
  REGION 1..263: note = misc_feature - UniProtKB - Q8BG84 (LAIR1_MOUSE), mouse LAIR-1 (mLAIR-1) consensus sequence for mLAIR-1a (isoform 1)
MSLHPVILLVLVLCLGWKINTQEGSLPDITIFPNSSLMISQGTFVTVVCSYSDKHDLYNMVRLEKDGSTFMEKSTEPYKTEDEFEIGPVNETITGHYSCIYSKGITWSERSKTLELKVIKENVIQTPAPGPTSDTSWLKTYSIYIFTVVSVIFLLCLSALLFCFLRHRQKKQGLPNNKRQQQRPEERLNLATNGLEMTPDIVADDRLPEDRWTETWTPVAGDLQEVTYIQLDHHSLTQRAVGAVTSQSTDMAESSTYAAIIRH

SEQ ID NO: 4 - protein, 32 aa, Mus musculus
  REGION 1..32: note = misc_feature - isoform 3
SLPDITIFPNSSLMISQGTFVTVVCSYSDKHD

SEQ ID NO: 5 - protein, 32 aa, Mus musculus
  REGION 1..32: note = misc_feature - mLAIR-1b (also known as isoform 2)
ELCLWFLLYPWATLELIMCTWDAWKETLEYFL

SEQ ID NO: 6 - protein, 123 aa, Mus musculus
  REGION 1..123: note = misc_feature - murine LAIR-1
QEGSLPDITIFPNSSLMISQGTFVTVVCSYSDKHDLYNMVRLEKDGSTFMEKSTEPYKTEDEFEIGPVNETITGHYSCIYSKGITWSERSKTLELKVIKENVIQTPAPGPTSDTSWLKTYSIY

SEQ ID NO: 7 - protein, 107 aa, synthetic construct
  REGION 1..107: note = Synthetic: 10D6 VL
DIQMTQSPASQSASLGESVTITCLASQTIGTWLAWFQQKPGKSPQLLIYAATSLADGVPSRFSGSGSGTKFSFKISSLQAEDFVSYYCQQLYSAPYTFGGGTKLEIK

SEQ ID NO: 8 - protein, 214 aa, synthetic construct
  REGION 1..214: note = Synthetic: c10D6 Light Chain (human kappa constant domain)
DIQMTQSPASQSASLGESVTITCLASQTIGTWLAWFQQKPGKSPQLLIYAATSLADGVPSRFSGSGSGTKFSFKISSLQAEDFVSYYCQQLYSAPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 117 aa, synthetic construct
  REGION 1..117: note = Synthetic: 10D6 VH
EVHLVETGGGLVQPKGSLKLSCAASGFNFNTNAMNWVRQAPGKGLEWVARIRTKSNNYATYYADSVKDRFTISRDDSQSMLYLQMNNLKTEDTAMYYCVSTPYFTYWGQGTLVTVSA

SEQ ID NO: 10 - protein, 446 aa, synthetic construct
  REGION 1..446: note = Synthetic: c10D6 Heavy Chain (human IgG1 constant domain)
EVHLVETGGGLVQPKGSLKLSCAASGFNFNTNAMNWVRQAPGKGLEWVARIRTKSNNYATYYADSVKDRFTISRDDSQSMLYLQMNNLKTEDTAMYYCVSTPYFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 443 aa, synthetic construct
  REGION 1..443: note = Synthetic: c10D6 Heavy Chain (human IgG4 constant domain)
EVHLVETGGGLVQPKGSLKLSCAASGFNFNTNAMNWVRQAPGKGLEWVARIRTKSNNYATYYADSVKDRFTISRDDSQSMLYLQMNNLKTEDTAMYYCVSTPYFTYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 107 aa, synthetic construct
  REGION 1..107: note = Synthetic: 11B3 VL
DIQMAQSSSSFSVSLGDRVTITCKASEDIYIRLAWYQQKPGNAPRLLISTATSLETGVPSRFSGSGSGKDYTLSITSLQTEDVATYYCQQYWSTPYTFGGGTRLEIK

SEQ ID NO: 13 - protein, 214 aa, synthetic construct
  REGION 1..214: note = Synthetic: c11B3 Light Chain (human kappa constant domain)
DIQMAQSSSSFSVSLGDRVTITCKASEDIYIRLAWYQQKPGNAPRLLISTATSLETGVPSRFSGSGSGKDYTLSITSLQTEDVATYYCQQYWSTPYTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 107 aa, synthetic construct
  REGION 1..107: note = Synthetic: h11B3 VL-1
DIQMTQSPSSLSASVGDRVTITCKASEDIYIRLAWYQQKPGKAPRLLISTATSLETGVPSRFSGSGSGTDYTFTISSLQPEDIATYYCQQYWSTPYTFGGGTRLEIK

SEQ ID NO: 15 - protein, 214 aa, synthetic construct
  REGION 1..214: note = Synthetic: h11B3 VL-1 Light Chain (kappa constant domain)
DIQMTQSPSSLSASVGDRVTITCKASEDIYIRLAWYQQKPGKAPRLLISTATSLETGVPSRFSGSGSGTDYTFTISSLQPEDIATYYCQQYWSTPYTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 107 aa, synthetic construct
  REGION 1..107: note = Synthetic: h11B3 VL-2
DIQMTQSPSSLSASLGDRVTITCKASEDIYIRLAWYQQKPGKAPRLLISTATSLETGVPSRFSGSGSGTDYTFTISSLQPEDIATYYCQQYWSTPYTFGGGTRLEIK

SEQ ID NO: 17 - protein, 214 aa, synthetic construct
  REGION 1..214: note = Synthetic: h11B3 VL-2 Light Chain (kappa constant domain)
DIQMTQSPSSLSASLGDRVTITCKASEDIYIRLAWYQQKPGKAPRLLISTATSLETGVPSRFSGSGSGTDYTFTISSLQPEDIATYYCQQYWSTPYTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 119 aa, synthetic construct
  REGION 1..119: note = Synthetic: 11B3 VH
EVQLVESGGGLVQPKGSLKLSCAASGFTFNTNAMYWVRQAPGKGLEWVARIRSKSSNYATYYADSVKDRFTISRDDSQSMLYLQMNNLKTEDTARYYCVRGGSGFFAYWGQGTLVTVSA

SEQ ID NO: 19 - protein, 445 aa, synthetic construct
  REGION 1..445: note = Synthetic: c11B3 Heavy Chain (human IgG1 constant domain)
EVQLVESGGGLVQPKGSLKLSCAASGFTFNTNAMYWVRQAPGKGLEWVARIRSKSSNYATYYADSVKDRFTISRDDSQSMLYLQMNNLKTEDTARYYCVRGGSGFFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 445 aa, synthetic construct; identical to SEQ ID NO: 19
  REGION 1..445: note = Synthetic: c11B3 Heavy Chain (human IgG4 constant domain)

SEQ ID NO: 21 - protein, 119 aa, synthetic construct
  REGION 1..119: note = Synthetic: h11B3 VH-1
EVQLVESGGGLVQPGGSLKLSCAASGFTFNTNAMYWVRQAPGKGLEWVARIRSKSSNYATYYAASVKGRFTISRDDSKNTLYLQMNSLKTEDTAVYYCVRGGSGFFAYWGQGTLVTVSS

SEQ ID NO: 22 - protein, 445 aa, synthetic construct
  REGION 1..445: note = Synthetic: h11B3 VH-1 Heavy Chain (IgG1 constant domain)
EVQLVESGGGLVQPGGSLKLSCAASGFTFNTNAMYWVRQAPGKGLEWVARIRSKSSNYATYYAASVKGRFTISRDDSKNTLYLQMNSLKTEDTAVYYCVRGGSGFFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 445 aa, synthetic construct; identical to SEQ ID NO: 22
  REGION 1..445: note = Synthetic: h11B3 VH-1 Heavy Chain (IgG4 constant domain)

SEQ ID NO: 24 - protein, 119 aa, synthetic construct
  REGION 1..119: note = Synthetic: h11B3 VH-2
EVQLVESGGGLVQPGGSLKLSCAASGFTFSTQAMYWVRQAPGKGLEWVARIRSKSSNYATYYAASVKGRFTISRDDSKNTLYLQMNSLKTEDTAVYYCVRGGSGFFAYWGQGTLVTVSS

SEQ ID NO: 25 - protein, 445 aa, synthetic construct
  REGION 1..445: note = Synthetic: h11B3 VH-2 Heavy Chain (IgG1 constant domain)
EVQLVESGGGLVQPGGSLKLSCAASGFTFSTQAMYWVRQAPGKGLEWVARIRSKSSNYATYYAASVKGRFTISRDDSKNTLYLQMNSLKTEDTAVYYCVRGGSGFFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 445 aa, synthetic construct; identical to SEQ ID NO: 25
  REGION 1..445: note = Synthetic: h11B3 VH-2 Heavy Chain (IgG4 constant domain)

SEQ ID NO: 27 - protein, 119 aa, synthetic construct
  REGION 1..119: note = Synthetic: h11B3 VH-3
EVQLVESGGGLVQPGGSLKLSCAASGFTFNTNAMYWVRQAPGKGLEWVGRIRSKSSNYATYYAASVKGRFTISRDDSKNTLYLQMNSLKTEDTAVYYCVRGGSGFFAYWGQGTLVTVSS

SEQ ID NO: 28 - protein, 445 aa, synthetic construct
  REGION 1..445: note = Synthetic: h11B3 VH-3 Heavy Chain (IgG1 constant domain)
EVQLVESGGGLVQPGGSLKLSCAASGFTFNTNAMYWVRQAPGKGLEWVGRIRSKSSNYATYYAASVKGRFTISRDDSKNTLYLQMNSLKTEDTAVYYCVRGGSGFFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 445 aa, synthetic construct; identical to SEQ ID NO: 28
  REGION 1..445: note = Synthetic: h11B3 VH-3 Heavy Chain (IgG4 constant domain)

SEQ ID NO: 30 - protein, 119 aa, synthetic construct
  REGION 1..119: note = Synthetic: h11B3 VH-4
EVQLVESGGGLVQPGGSLKLSCAASGFTFNTNAMYWVRQAPGKGLEWVARIRSKSSNYATYYADSVKGRFTISRDDSKNTLYLQMNSLKTEDTAVYYCVRGGSGFFAYWGQGTLVTVSS

SEQ ID NO: 31 - protein, 445 aa, synthetic construct
  REGION 1..445: note = Synthetic: h11B3 VH-4 Heavy Chain (IgG1 constant domain)
EVQLVESGGGLVQPGGSLKLSCAASGFTFNTNAMYWVRQAPGKGLEWVARIRSKSSNYATYYADSVKGRFTISRDDSKNTLYLQMNSLKTEDTAVYYCVRGGSGFFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 32 - protein, 445 aa, synthetic construct; identical to SEQ ID NO: 31
  REGION 1..445: note = Synthetic: h11B3 VH-4 Heavy Chain (IgG4 constant domain)

SEQ ID NO: 33 - protein, 119 aa, synthetic construct
  REGION 1..119: note = Synthetic: h11B3 VH-5
EVQLVESGGGLVQPGGSLKLSCAASGFTFNTNAMYWVRQAPGKGLEWVGRIRSKSSNYATYYADSVKGRFTISRDDSKNTLYLQMNSLKTEDTAVYYCVRGGSGFFAYWGQGTLVTVSS

SEQ ID NO: 34 - protein, 445 aa, synthetic construct
  REGION 1..445: note = Synthetic: h11B3 VH-5 Heavy Chain (IgG1 constant domain)
EVQLVESGGGLVQPGGSLKLSCAASGFTFNTNAMYWVRQAPGKGLEWVGRIRSKSSNYATYYADSVKGRFTISRDDSKNTLYLQMNSLKTEDTAVYYCVRGGSGFFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 35 - protein, 445 aa, synthetic construct; identical to SEQ ID NO: 34
  REGION 1..445: note = Synthetic: h11B3 VH-5 Heavy Chain (IgG4 constant domain)

SEQ ID NO: 36 - protein, 119 aa, synthetic construct
  REGION 1..119: note = Synthetic: h11B3 VH-6
EVQLVESGGGLVQPGGSLKLSCAASGFTFSTNAMYWVRQAPGKGLEWVGRIRSKSSNYATYYADSVKGRFTISRDDSKNTLYLQMNSLKTEDTAVYYCVRGGSGFFAYWGQGTLVTVSS

SEQ ID NO: 37 - protein, 445 aa, synthetic construct
  REGION 1..445: note = Synthetic: h11B3 VH-6 Heavy Chain (IgG1 constant domain)
EVQLVESGGGLVQPGGSLKLSCAASGFTFSTNAMYWVRQAPGKGLEWVGRIRSKSSNYATYYADSVKGRFTISRDDSKNTLYLQMNSLKTEDTAVYYCVRGGSGFFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 38 - protein, 445 aa, synthetic construct; identical to SEQ ID NO: 37
  REGION 1..445: note = Synthetic: h11B3 VH-6 Heavy Chain (IgG4 constant domain)

SEQ ID NO: 39 - protein, 119 aa, synthetic construct
  REGION 1..119: note = Synthetic: h11B3 VH-7
EVQLVESGGGLVQPGGSLKLSCAASGFTFNTQAMYWVRQAPGKGLEWVGRIRSKSSNYATYYADSVKGRFTISRDDSKNTLYLQMNSLKTEDTAVYYCVRGGSGFFAYWGQGTLVTVSS

SEQ ID NO: 40 - protein, 445 aa, synthetic construct
  REGION 1..445: note = Synthetic: h11B3 VH-7 Heavy Chain (IgG1 constant domain)
EVQLVESGGGLVQPGGSLKLSCAASGFTFNTQAMYWVRQAPGKGLEWVGRIRSKSSNYATYYADSVKGRFTISRDDSKNTLYLQMNSLKTEDTAVYYCVRGGSGFFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 445 aa, synthetic construct; identical to SEQ ID NO: 40
  REGION 1..445: note = Synthetic: h11B3 VH-7 Heavy Chain (IgG4 constant domain)

SEQ ID NO: 42 - protein, 119 aa, synthetic construct
  REGION 1..119: note = Synthetic: h11B3 VH-8
EVQLVESGGGLVQPGGSLKLSCAASGFTFSTQAMYWVRQAPGKGLEWVGRIRSKSSNYATYYADSVKGRFTISRDDSKNTLYLQMNSLKTEDTAVYYCVRGGSGFFAYWGQGTLVTVSS

SEQ ID NO: 43 - protein, 445 aa, synthetic construct
  REGION 1..445: note = Synthetic: h11B3 VH-8 Heavy Chain (IgG1 constant domain)
EVQLVESGGGLVQPGGSLKLSCAASGFTFSTQAMYWVRQAPGKGLEWVGRIRSKSSNYATYYADSVKGRFTISRDDSKNTLYLQMNSLKTEDTAVYYCVRGGSGFFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 445 aa, synthetic construct; identical to SEQ ID NO: 43
  REGION 1..445: note = Synthetic: h11B3 VH-8 Heavy Chain (IgG4 constant domain)

(19) United States
(12) Patent Application Publication
(10) Pub. No.: US 2024/0352121 A1
(43) Pub. Date: Oct. 24, 2024

(54) LAIR-1 AGONISTIC ANTIBODIES AND METHODS OF USE THEREOF

(71) Applicant: NEXTCURE, INC., Beltsville, MD (US)

(72) Inventors: Rustin LOVEWELL, Beltsville, MD (US); Dallas Benjamin FLIES, Beltsville, MD (US); Zachary CUSUMANO, Beltsville, MD (US); Solomon LANGERMANN, Beltsville, MD (US); Han MYINT, Beltsville, MD (US)

(21) Appl. No.: 18/444,472

(22) Filed: Feb. 16, 2024

Related U.S. Application Data
(60) Provisional application No. 63/485,848, filed on Feb. 17, 2023.

(51) Int. Cl. C07K 16/28; A61K 31/496; A61K 31/706; A61K 38/39; A61P 35/02

(52) U.S. Cl. CPC C07K 16/2803; A61K 31/496; A61K 31/706; A61K 38/39; A61P 35/02; C07K 2317/565

(57) ABSTRACT

A pharmaceutical composition for effectively depleting cancer cells while sparing normal, healthy cells. The pharmaceutical composition may include a humanized monoclonal antibody that binds LAIR-1 in an agonistic fashion. Said composition can also include additional therapeutic agents, which act synergistically to treat cancers including but not limited to acute myeloid leukemia (AML). Compositions can be administered through a variety of methods to treat a subject in need thereof.

### REFERENCE TO RELATED APPLICATIONS

This application claims the benefit of priority to U.S. Provisional Application Ser. No. 63/485,848, filed Feb. 17, 2023, which is hereby incorporated by this reference in its entirety.

### REFERENCE TO ELECTRONIC SEQUENCE LISTING

The application contains a Sequence Listing which has been submitted electronically in .XML format and is hereby incorporated by reference in its entirety. Said. XML copy, created on Apr. 24, 2024, is named “064467.083US1.xml” and is 68,839 bytes in size. The sequence listing contained in this .XML file is part of the specification and is hereby incorporated by reference herein in its entirety.

### TECHNICAL FIELD

This disclosure generally relates to the field of immunotherapy, and more particularly to compositions and methods of using LAIR-1 antibodies.

### BACKGROUND

Acute leukemias are characterized by uncontrolled production of malignant hematopoietic progenitors. Acute myeloid leukemia (AML) is the most common adult acute leukemia (ACS, Cancer Facts and Figures 2022). While extensive research has led to the recent approval of novel therapies for AML (Stanchina 2020), there remains significant unmet need for patients that fail to respond to or relapse after standard-of-care (SoC) treatments. This may be in large part due to the persistence of leukemia stem cells (LSCs) (van Gils et al., 2021).

LSCs are leukemia initiating cells at the apex of the hierarchy of leukemia cells in the bone marrow with the ability for self-renewal. LSCs give rise to daughter leukemic blasts, initiate leukemic disease when transplanted to immunodeficient animals, and propagate upon serial transplantation (Majeti et al 2017). The self-renewal capacity of LSCs leads to recurrence and relapse in 50% of all patients who achieved remission after initial treatment (Yilmaz et al., 2019), making LSCs a critical target for next generation therapeutics in AML.

Leukocyte Associated Immunoglobulin Like Receptor 1 (LAIR-1) is an immunoglobulin superfamily protein with two Immunoreceptor Tyrosine-based Inhibitory Motifs (ITIMs) that can recruit Src homology region 2 domain-containing phosphatase-1 (SHP-1) and C-terminal Src Kinase (CSK) (Meyaard 2006, Meyaard 2008). LAIR-1 is restricted to the hematopoietic compartment, particularly myeloid cells, but also T cells, B cells, and NK cells (Meyaard 2008). The LAIR-1 IgV domain is unique in its ability to specifically bind collagen domain-containing ligands, including collagens, complement protein C1q, Surfactant Protein-D (SP-D), Mannose Binding Lectin (MBL), and Collectin-12 (Meyaard, 2008, Keerthivasan S, 2021). When ligands bind to LAIR-1, receptor clustering results in phosphorylation of LAIR-1 ITIM domains which recruit and phosphorylate SHP-1 phosphatases to trigger downstream immune-inhibitory signaling (Meyaard 2008). LAIR-1 has limited or redundant function in healthy cells and homeostatic environments but dampens immune responses in non-homeostatic or disease environments (Meyaard 2010, Son and Diamond 2014, Jin et al., 2018).

More recently, LAIR-1 function has been linked to cell stemness and disease development in leukemia (Kang, Lu et al., 2015). Ligand independent LAIR-1 constitutive phosphorylation and signaling and SHP-1 phosphorylation independent pathways have been described (Kang, Lu et al., 2015), underscoring the importance of context for LAIR-1 function. Specifically, LAIR-1 on AML cells can lead to downstream signaling through Ca++/calmodulin-dependent protein kinase (CAMK1) and cAMP response element-binding protein (CREB) (Kang, Lu et al., 2015), which has been implicated in sustaining AML stem cell activity (Kang, Lu et al., 2015; Kang, Kim et al., 2016). While LAIR-1 is dispensable for normal hematopoiesis (Tang et al., 2012; Kang, Lu et al., 2015), knockdown of LAIR-1 in human leukemia cells increased apoptosis in vitro and reduced AML development in murine models (Kang, Lu et al., 2015).

In contrast, a separate study found that LAIR-1 ligation on leukemia cells inhibits IκBα activation to prevent nuclear factor kappa B (NF-κB) translocation into the nucleus, resulting in programmed cell death (Poggi et al., 2000). Indeed, follow-up studies showed that receptor clustering blocked proliferation of AML blasts and led to subsequent cellular apoptosis, an effect which was dependent on LAIR-1 ITIM signal transduction through SHP-1 (Zocchi et al., 2001).

There remains a need in the art to provide improved methods and compositions for selectively targeting LAIR-1 for treatment of various cancers.

### SUMMARY

It is to be understood that this summary is not an extensive overview of the disclosure. This summary is exemplary and not restrictive and it is intended to neither identify key or critical elements of the disclosure nor delineate the scope thereof. The sole purpose of this summary is to explain and exemplify certain concepts of the disclosure as an introduction to the following complete and extensive detailed description.

The present disclosure relates to a pharmaceutical composition containing an immunomodulatory agent. The immunomodulatory agent is capable of binding LAIR-1 as an agonist. In doing so, the composition effectively treats cancer by depleting cancer cells while sparing normal, healthy cells. As a non-limiting example, cancer cells include leukemia stem cells (LSCs) and leukemic blasts. As a further non-limiting example, healthy cells include normal hematopoietic stem cells. The agent can be used to treat all solid and hematologic tumors. The agent can be an antibody where the antibody can be a humanized monoclonal antibody. A humanized monoclonal antibody can be selected as LAIR-1 mAb.

The present disclosure relates to a humanized monoclonal antibody with a variable light chain comprising at least 95%, 96%, 97%, 98%, 99%, or 100% sequence identity to a variable light chain having an amino acid sequence selected from the group of variable light chains consisting of SEQ ID NOs: 6, 7, 11, 13, 14, 15, and 16. One aspect of the invention presents a LAIR-1 antibody comprising a variable light chain domain having 95%, 96%, 97%, 98%, 99% and 100% sequence identity to sequences selected from the group consisting of SEQ ID NOs: 14 and 16. Another aspect presents a LAIR-1 antibody comprising a light chain having 95%, 96%, 97%, 98%, 99% and 100% sequence identity to sequences selected from the group consisting of SEQ ID Nos: 15 and 17.

The present disclosure relates to a humanized monoclonal antibody with a variable heavy chain comprising at least 95%, 96%, 97%, 98%, 99%, or 100% sequence identity to a variable heavy chain having an amino acid sequence selected from the group of variable heavy chains consisting of SEQ ID NOs: 8, 17, 20, 21, 22, 23, 24, 25, 26, 27, 28, 29, 30, 31, 32, 33, 34, 35, 36, 37, 38, 39, 40, 41, 42, and 43. One aspect presents a LAIR-1 antibody comprising a variable heavy chain domain having 95%, 96%, 97%, 98%, 99% and 100% sequence identity to sequences selected from the group consisting of SEQ ID NOs: 21, 24, 27, 30, 33, 36, 39 and 42. Another aspect presents a LAIR-1 antibody comprising a heavy chain having 95%, 96%, 97%, 98%, 99% and 100% sequence identity to sequences selected from the group consisting of SEQ ID Nos: 22, 23, 25, 26, 28, 29, 31, 32, 34, 35, 37, 38, 40, 41, 43 and 44.

The present disclosure relates to a humanized monoclonal antibody with a variable light chain comprising at least 95%, 96%, 97%, 98%, 99%, or 100% sequence identity to a variable light chain having an amino acid sequence selected from the group of variable light chains consisting of SEQ ID NOs: 6, 7, 11, 13, 14, 15, and 16 and also with a variable heavy chain comprising at least 95%, 96%, 97%, 98%, 99%, or 100% sequence identity to a variable heavy chain having an amino acid sequence selected from the group of variable heavy chains consisting of SEQ ID NOs: 8, 17, 20, 21, 22, 23, 24, 25, 26, 27, 28, 29, 30, 31, 32, 33, 34, 35, 36, 37, 38, 39, 40, 41, 42, and 43.

One other aspect presents a LAIR-1 antibody comprising a variable light chain domain and a variable heavy chain domain, wherein the variable light chain domain and variable heavy chain domain have 95%, 96%, 98%, 99% and 100% sequence identity to sequences selected from the group consisting of SEQ ID NOs: 14 and 21, 14 and 24, 14 and 27, 14 and 30, 14 and 33, 14 and 36, 14 and 39, 14 and 42, 16 and 21, 16 and 24, 16 and 27, 16 and 30, 16 and 33, 16 and 36, 16 and 39, and 16 and 42.

Another aspects presents a LAIR-1 antibody comprising a light chain and a heavy chain, wherein the light chain and heavy chain have 95%, 96%, 98%, 99% and 100% sequence identity to sequences selected from the group consisting of SEQ ID NOs: 15 and 22, 15 and 23, 15 and 25, 15 and 26, 15 and 28, 15 and 29, 15 and 31, 15 and 32, 15 and 34, 15 and 35, 15 and 37, 15 and 38, 15 and 40, 15 and 41, 15 and 43, 15 and 44, 17 and 22, 17 and 23, 17 and 25, 17 and 26, 17 and 28, 17 and 29, 17 and 31, 17 and 32, 17 and 34, 17 and 35, 17 and 37, 17 and 38, 17 and 40, 17 and 41, 17 and 43, and 17 and 44.

The present disclosure relates to a humanized monoclonal antibody with a heavy chain comprising at least 95%, 96%, 97%, 98%, 99%, or 100% sequence identity to a heavy chain having an amino acid sequence selected from the group of heavy chains consisting of SEQ ID NOs: 9, 10, 18, and 19. In one aspect, the invention presents a LAIR-1 antibody comprising a variable heavy chain domain having 95%, 96%, 98%, 99% and 100% sequence identity to SEQ ID NO: 9. Another aspect presents a LAIR-1 antibody comprising the antigen binding domain having 95%, 96%, 98%, 99% and 100% sequence identity to SEQ ID NO: 9.

The present disclosure relates to a humanized monoclonal antibody with a light chain comprising at least 95%, 96%, 97%, 98%, 99%, or 100% sequence identity to a light chain having an amino acid sequence selected from the group of light chains consisting of SEQ ID NOs: 7 and 12.

The present disclosure relates to a humanized monoclonal antibody with a light chain comprising at least 95%, 96%, 97%, 98%, 99%, or 100% sequence identity to a light chain having an amino acid sequence selected from the group of light chains consisting of SEQ ID NOs: 7 and 12 and a heavy chain comprising at least 95%, 96%, 97%, 98%, 99%, or 100% sequence identity to a heavy chain having an amino acid sequence selected from the group of heavy chains consisting of SEQ ID NOs: 9, 10, 18, and 19.

One other aspect presents a LAIR-1 antibody comprising a variable light chain domain having 95%, 96%, 98%, 99% and 100% sequence identity to SEQ ID NO: 7. Another aspect presents a LAIR-1 antibody comprising the antigen binding domain having 95%, 96%, 98%, 99% and 100% sequence identity to SEQ ID NO: 7.

The present disclosure relates to a pharmaceutical composition including an immunomodulatory agent, as disclosed herein, in addition to one or more therapeutic agents. As non-limiting examples, therapeutic agents include venetoclax, azacytidine and CD47 antibodies. The combination of an immunomodulatory agent and one or more therapeutic agents has a synergistic effect in treating cancers including but not limited to acute myeloid leukemia (AML). On one embodiment, the invention presents a combination of therapeutic agents, wherein the therapeutic agents comprise any of the LAIR-1 antibodies of any one of the above sequences, in combination with venetoclax, azacytidine and CD47 antibodies.

The present disclosure relates to methods of treating a subject in need thereof by administering a pharmaceutical composition with an effective amount of an immunomodulatory agent for binding LAIR-1, as disclosed herein. One aspect presents a method of treating a subject in need thereof by administering a pharmaceutical composition comprising a LAIR-1 antibody and any one of the above sequences. In one embodiment, the subject to be treated has carcinoma, squamous cell carcinoma, leukemia, acute lymphocytic leukemia, acute lymphoblastic leukemia, B-cell lymphoma, T-cell lymphoma, Berketts lymphoma, acute myelogenous leukemia, chronic myelogenous leukemia, promyelocytic leukemia, fibrosarcoma, rhabdomyoscarcoma, melanoma, seminoma, tetratocarcinoma, neuroblastoma, glioma, astrocytoma, neuroblastoma, glioma, schwannomas, fibrosarcoma, rhabdomyoscarama, osteosarcoma, xenoderma pegmentosum, keratoactanthoma, seminoma, thyroid follicular cancer, or teratocarcinoma. In another embodiment, the subject to be treated has acute myeloid leukemia. Administration can occur through any method known in the art including but not limited to parenteral, oral, and topical administration.

The present disclosure relates to kits of compositions disclosed herein. Kits may include one or more agents including but not limited to a LAIR-1 antibody of any one of the sequences presented herein, venetoclax, azacytidine and anti-CD47, or the combination thereof.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIGS. 1A-1G display a target acute myeloid leukemia (AML) therapy. FIG. 1A displays LAIR-1 transcript levels, as measured by RNA-Seq and quantified by RSEM software, in AML patient samples clustered by French-American-British (FAB) classification or by FIG. 1B displays molecular mutation. UD=undiagnosed (non-AML), N=1-47 patient samples per group. FIG. 1C displays an illustration of leukemopoiesis from leukemic stem cell (LSC) precursors into granulocyte-monocyte progenitor (GMP-like) LSCs, lymphoid primed multipotent progenitor (LMP-like) LSCs, or multipotential progenitor (MPP-like) LSCs. FIG. 1D displays mean fluorescence intensity of LAIR-1 cell surface expression on the indicated LSC subpopulations. FIG. 1E displays an illustration of normal hematopoiesis from healthy stem cells (HSCs) into multipotential progenitors (MPPs), common lymphoid progenitors (CLPs), common myeloid progenitors (CMPs), or granulocyte-monocyte progenitors (GMPs). FIG. 1F displays mean fluorescence intensity of LAIR-1 cell surface expression on the indicated HSC subpopulations. FIG. 1G displays a comparison of LAIR-1 cell surface expression on cells from comparable compartments from AML patients or healthy donors (H). Each dot represents a unique donor/patient. n=7 healthy donors or 25 AML patients. P value determined by Student's/test. Data are shown as the mean±SEM.

FIGS. 2A-2C display LAIR-1 engagement effectively depletes LSCs while sparing normal hematopoiesis. FIG. 2A displays representative images of LSC ex vivo colony formation under the indicated treatment dosage. FIG. 2B displays colony forming units (CFUs) formed by ex vivo plating of LSCs from the indicated AML donor during titrated treatment with anti-LAIR-1 agonist mAb. N=3 technical replicates per group. P values determined by one-way ANOVA with multiple comparisons. FIG. 2C displays CFU formation from healthy donor bone-marrow or AML patient bone-marrow treated with 5 μg/mL LAIR-1 mAb. Values normalized to isotype control. N=5 healthy biological replicates or 14 AML biological replicates. P value determined by Student's T test. Error bars represent standard error of mean.

FIGS. 3A-3C display LAIR-1 engagement eradicating primary and secondary AML in patient derived xenograft models. FIG. 3A displays a schematic of AML patient derived xenograft (PDX) model and representative scatterplots of human (H) CD33 CD45 leukemic cells in circulation at the indicated time post-engraftment. FIG. 3B displays Leukemic growth, as measured by the percent of circulating _{H}CD33⁺ _{H}CD45⁺ cells, in PDX mice engrafted with bone-marrow from donors with normal karyotype AML, monocytic AML, acute myelomonocytic leukemia (AMML), Flt3 ITD⁺ AML, or uncharacterized AML. Engrafted mice were treated with 5 mg/kg IgG isotype control (gray) or LAIR-1 mAb (red). N=3-5 mice per group. FIG. 3C displays a schematic of PDX secondary transplant model, where bone-marrow from PDX mice engrafted and treated as above was harvested and secondarily transplanted into tumor and treatment naïve recipient mice. Graphs show leukemic growth in secondary recipient mice after receiving bone-marrow from AMML PDX animals (top) or normal karyotype AML PDX animals (bottom) that had been treated with 5 mg/kg IgG isotype control (gray) or LAIR-1 mAb (red). N=3 mice per group. P values calculated by two-way ANOVA. Error bars represent standard error of mean.

FIGS. 4A-4O show the collagen matrix is vital to LAIR-1 induced AML cell death. (FIGS. 4A-C). Flow cytometry quantification of primary live, dead, or apoptotic total blood leukocytes after ex vivo treatment with 10 μg/mL IgG isotype control (gray) or LAIR-1 mAb (red) in the presence (FIG. 4A) or absence (FIG. 4B) of exogenous collagen, or (FIG. 4C), CD451^{Lo} SSC^{Lo} blast cells in the presence of isotype control or LAIR-1 mAb. N=3 technical replicates. FIG. 4D shows percentage of dead cells from whole blood leukocytes (left) or CD45^{Lo} SSC^{Lo} blasts (right) after treatment with 10 μg/mL LAIR-1 mAb (values normalized to isotype control) in the presence of collagen, graphed as a function of LAIR-1 surface expression. Each dot represents an individual donor. Red shading highlights surface expression >20000 arbitrary units. Blue shading highlights expression <20000 units. Line represents simple linear regression. FIG. 4E shows total live cells (left) or CD45^{Lo} SSC^{Lo} Blast cells (right) from healthy blood donors or AML patient donors after ex vivo treatment with 10 μg/mL LAIR-1 mAb (values normalized to isotype control) in the presence of collagen. N=4-7 donors. FIG. 4F shows phosphorylated SHP-1 in AML patient PBMCs cultured under the indicated conditions. Mean pixel density from Western blotting normalized to isotype control condition and graphed relative to total SHP-1. N=3 technical replicates. FIGS. 4G and 4H show mean pixel density from phospho-array of AML patient PBMCs treated ex vivo with 10 μg/mL of IgG isotype control (gray), 50 μg/mL coated collagen (white), or 10 μg/mL LAIR-1 mAb+coated collagen (red) for G, MAPK activity or (FIG. 4H), mTOR and NF-kB activity. N=2 technical replicates. FIG. 4I shows schematic of LAIR-1 mAb crosslinking and LAIR-1 clustering using anti-IgG to crosslink the Fc-domain of LAIR-1 mAb when bound to cell surface LAIR-1. FIG. 4J shows in vitro growth of MV4-11-LAIR-1^{Overexpressing} cells treated with 10 μg/mL IgG isotype control (gray) or LAIR-1 mAb (red) in the absence or (FIG. 4K), presence of anti-IgG crosslinking. FIG. 4L shows Annexin V staining of cells treated as in FIGS. 4I-4K, with representative scatterplot of apoptotic annexin V+ Live-Dead Aqua− cells at day 3 of culture. FIGS. 4M and 4N) 4E-BP1 expression as measured by Lumit assay (FIG. 4M) or cleaved caspase-7 as measured by Western blot (FIG. 4N) of cells treated as shown in FIGS. 4I-4K. Mean pixel density normalized to histone-3 for triplicate samples. FIG. 4O shows the percentage TUNEL+MV4-11-LAIR-1^{overexpressing} cells at day 3 of treatment plus DMSO vehicle, 50 μM 220509-74-0 (caspase-3/7 inhibitor), or 50 μM MHY1485 (mTOR activator). Values normalized to isotype for each respective condition. n=3-4 technical replicates. P values calculated by Student's t test. Error bars represent SEM.

FIGS. 5A-5I display LAIR-1 engagement by LAIR-1 mAb systemically reduces AML growth that is dependent on LAIR-1 expression level, but does not require or effect immune cells. FIG. 5A displays a schematic of the MV4-11-luciferase or THP-1-luciferase cell derived xenograft (CDX) model of AML. FIG. 5B displays in vivo leukemic growth as measured by whole body luminescence of MV4-11-luciferase cells (left) or THP-1-luciferase cells (right) in CDX mice treated with 10 mg/kg IgG isotype control (gray) or LAIR-1 mAb (red). N=8 mice per group. P values determined by two-way ANOVA. FIG. 5C displays MV4-11 cell counts or (FIG. 5D), percent dead MV4-11 cells in the blood, spleen, or bone-marrow of CDX mice treated with vehicle control (gray) or 10 μg/mL LAIR-1 mAb (red). FIG. 5E displays total cell counts or mouse (_{M}) CD45 cell counts in the bone marrow of CDX mice treated with vehicle control (gray) or 10 μg/mL LAIR-1 mAb (red). N=9-10 mice per group. FIG. 5F displays representative histograms of LAIR-1 cell surface expression on the indicated cell lines. FIG. 5G displays a schematic of CDX model systems to test inhibition of leukemic growth as a function of LAIR-1 expression. FIG. 5H displays (left) percent inhibition of MV4-11-LAIR-1-knockout (green), MV4-11-LAIR-1-wildtype (purple), or MV4-11-LAIR-1-overexpression cell growth in vivo (normalized to the respective isotype controls) after treatment with 10 mg/kg mL LAIR-1 mAb and (right) plotted against LAIR-1 geometric mean fluorescence intensity. FIG. 5I displays percent inhibition of MV4-11 growth fit to a logarithmic regression curve of LAIR-1 expression. P values determined by Student's T test. Error bars represent standard error of mean.

FIGS. 6A-6D display LAIR-1 signaling restricts AML survival signaling pathways in vivo. FIG. 6A displays a schematic of in vivo MV4-11 subcutaneous model and line graph tumor growth in mice treated with 10 mg/kg isotype control (gray) or anti-LAIR-1 agonist mAb LAIR-1 mAb (red). N=3 mice per group. FIG. 6B displays a model schematic and a line graph MV4-11-luciferase growth in CDX mice used for digital spatial imaging and target protein quantification. N=6 mice per group. FIG. 6C is representative images of CDX mouse bones stained with DAPI and anti-human CD45 used to quantify the number of MV4-11 cells in the indicated region of interest (ROI) (highlighted). ROI area is equal between samples. FIG. 6D displays protein reads of human CD45, BCL-XL, or uncleaved PARP from bone or spleen harvested from CDX mice at day 22 post-engraftment. Read counts are normalized to Histone H3 and ribosomal protein S6. N=3-11 quantified tissue regions across 2 isotype-treated or 2 LAIR-1 mAb-treated mice. Error bars represent standard error of mean. P values determined by Student's T test.

FIGS. 7A-7G show that NC525 synergizes with AML standard-of-care therapy. Ex vivo killing of VEN/AZA-treated AML patient leukemic cells (FIG. 7A) or T cells or NK cells (FIG. 7B). Values normalized to vehicle control. Lines represent linear regression of NC525 concentration versus normalized cell killing. FIG. 7C shows LAIR-1 surface expression of VEN/AZA-treated AML patient leukemic cells, T cells, or NK cells. FIGS. 7D and 7E show in vivo leukemic growth as measured by whole-body luminescence of MV4-11-luciferase cells (FIG. 7D) or survival (FIG. 7E) of CDX mice treated with 10 mg/kg isotype control (gray) or NC525 (red) or 100 mg/kg VEN (pink) or 0.5 mg/kg AZA (blue) or combination therapy (green or yellow, respectively). BLoD, below limit of detection. n=9 mice per group. P values determined by 2-way ANOVA or log-rank (Mantel-Cox) test, respectively. FIGS. 7F and 7G show leukemic growth in the blood (FIG. 7F) and in the spleen and BM (FIG. 7G) at week 8 after transplant of AML PDX mice treated with vehicle (gray), VEN/AZA (blue), NC525 (red), or VEN/AZA plus NC525 (green). n=5-10 mice per group. AML cells in spleen and BM compared from 4-5 mice per group. Data are shown as the mean±SEM. P values determined by 2-way ANOVA or 1-way ANOVA with multiple comparisons.

FIG. 8 is a schematic of LAIR-1 induced cell death in leukemic cells. Engagement of LAIR-1 on AML cells by agonist mAb LAIR-1 mAb induces an inhibitory signal that blocks aberrant mTOR activity, leading to the suppression of constitutively active MAPK signaling and the self-renewal mechanisms promoted by AKT and NF-kB. This loss of proliferative signaling induces the de-activation of BCL-XL, which releases an apoptotic cascade through caspase-7 and PARP, culminating in programmed cell death.

FIG. 9 displays LAIR-1 cell surface expression analysis. Gating schematic for quantification of LAIR-1 on primary patient bone-marrow cell subpopulations.

FIGS. 10A-10F display characterization of LAIR-1 agonist monoclonal antibody. FIG. 10A displays (left) a schematic of human (_{H}) LAIR-1 reporter cell line UT-140. UT-140 cells that express GFP under the NFAT promoter were transduced with human LAIR-1 fused to the zeta chain of CD3. Upon LAIR-1 engagement, signal transduction activates GFP fluorescence. FIG. 10B displays a binding profile of LAIR-1 mAb to LAIR-1 UT-140 cells. FIG. 10C displays a binding profile of LAIR-1 mAb-Parent mAb to cell surface expressed mouse (M) LAIR-1. FIG. 10D displays a profile of LAIR-1 ligand collagen-1 blockade by LAIR-1 mAb measured by UT-140 reporter cell activation. Isotype treatment is indicated by gray circles; LAIR-1 mAb-parent mAb treatment is indicated by red squares.

FIG. 10E displays activation profiles of UT-140 LAIR-1 reporter cells by LAIR-1 mAb or collagen under the indicated conditions. FIG. 10F displays (left) a Western blot and (right) quantification (pixel density normalized to histone H3) for phosphorylated SHP-1 in healthy donor blood monocytes treated under the indicated conditions. Each line represents an individual donor. The collagen matrix is vital to LAIR-1 induced AML cell death.

FIGS. 11A-11C display representative flow cytometry gating and scatterplots of primary live, dead, or apoptotic total blood leukocytes after ex vivo treatment with 10 μg/mL IgG isotype control (gray) or LAIR-1 mAb (red) in the (FIG. 11A) presence or (FIG. 11B) absence of exogenous collagen, or (FIG. 11C) CD45^{Lo} SSC^{Lo} blast cells in the presence of isotype control or LAIR-1 mAb.

FIGS. 12A-12B display LAIR-1 mAb and collagen induced phosphorylation signaling. FIG. 12A displays human phospho-kinase array dot blots, and FIG. 12B displays human phospho-immunoreceptor array dot blots with the respective keys for AML patient PBMCs treated with 10 μg/mL isotype control, LAIR-1 agonist mAb, 50 μg/mL collagen-1 and isotype control, or collagen-1 and LAIR-1 mAb.

FIG. 13 displays LAIR-1 expression on AML cell lines. Histograms of LAIR-1 cell surface expression (blue) on the indicated AML cell lines relative to isotype control staining (red).

FIGS. 14A-14B display LAIR-1 monoclonal antibody does not impact healthy leukocytes. FIG. 14A displays a schematic of model system for defining LAIR-1 agonist mAb effects on human (_{H}) immune cells in vivo. FIG. 14B displays cell counts of human CD45 cells or human CD3 cells in the spleen or bone-marrow of engrafted mice treated with vehicle (gray) or 10 mg/kg anti-LAIR-1 agonist mAb. N=7 mice per group. P values determined by Student's T test. Error bars represent standard error of mean.

FIG. 15 shows LAIR-1 promotes cell survival in Acute Myeloid Leukemia (AML). U937-RF-luc LAIR1 wildtype or knockout and MV-4-11-RF-luc LAIR1 WT or KO cells were cultured at 1e5/well for 48 hrs. 10 μL of the XTT mix was added and cells were incubated for an additional 4 hrs. Absorbance was measured at 450 nm as measure or cell proliferation.

FIGS. 16A-16B display LAIR-1 mAb and anti-AML mechanisms. FIG. 16A displays LAIR-1-mediated LSC and blast survival where LAIR-1 is expressed on AML cells and interacts with natural ligands (C1Q, collagens) to promote the survival of AML LSC and blasts. FIG. 16B displays multimodal LAIR-1 mAb anti-leukemic activity where a LAIR-1 mAb blockade of natural ligand binding to AML cells disrupts survival signal and LAIR-1 mAb induces AML killing through Fc receptor dependent mechanisms including ADCP and ADCC.

FIGS. 17A-17C display aspects of LAIR-1 being highly expressed in AML blast and Leukemia Stem Cells (LSCs). FIG. 17B displays LAIR-1 mRNA expressions not varying among AML subsets with LAIR-1 mRNA expression in peripheral blood AML blasts according to disease subtypes defined by FAB classification, and mutation status. UD denotes undetermined. FIG. 17C displays LAIR-1 being highly expressed in blast/LSC but not in HSPC. FIG. 17C shows flow-cytometry based analysis of LAIR-1 protein expression on the cell surface of leukemic stem cells (LSCs; CD34+CD38−CD90−CD45RA+/− or CD34-CD117+CD244+/−) vs hematopoietic stem and progenitor cells (HSPCs; CD34+CD38-CD90+CD99−) derived from bone marrow aspirates, and leukemic blasts from the peripheral blood of AML patients.

FIGS. 18A-18B display LAIR-1 expression being lower in healthy donors than AML. FIG. 18A displays interactions with hematopoietic stem cells (HSCs). FIG. 18B displays that LAIR-1 is expressed in the AML blast gate (SSC^{lo} CD45^{lo}) in both the AML and heathy whole blood samples and that healthy donors' progenitors are mostly CD34− with less LAIR-1 than AML (MFI). LAIR1 protein levels were measured by flow cytometry.

FIGS. 19A-19B display LAIR-1 is expressed CD33+/− CD34+/− myeloid progenitors in AML bone marrow. In FIG. 19A, frozen AML patient bone marrow cells were thawed and stained with CD33, CD34 and LAIR-1 antibodies to determine expression using flow cytometry. All myeloid progenitors and leukemic stem cells from AML bone marrow CD33 and CD34 populations showed expression of LAIR-1. LAIR-1 was most highly expressed on CD33+CD34− population.

FIGS. 20A-20D display humanized 11B3 LAIR-1 mAbs exhibiting potent binding and signaling blockades. The h11B3 LAIR-1 mAbs include a variable light chain of SEQ ID NO: 16 and a variable heavy chain of SEQ ID NO:27, as shown for Anti-LAIR-1 Variant 6 in Table 1 herein. Such LAIR-1 mAbs display in vitro activity with K_{D}=2.1 nM, cross reactive to Cyno=1.2 nM, cell binding EC₅₀=0.15 nM, and blocks signaling IC₅₀=0.25 nM. In vivo activity includes efficacy in therapeutic NSG murine model challenged with AML cell lines MV-411 and THP-1, efficacy in therapeutic NSG-SGM3 murine model challenged with human primary AML cells, and selectivity for leukemic cells with no depletion, inhibition, or expansion of healthy human primary immune cells in NSG-SGM3 murine model. FIG. 20A displays binding to human LAIR-1 cells. FIG. 20B displays binding to cyno LAIR-1 cells. FIG. 20C displays a ligand-mediated signaling blockade. FIG. 20D displays a ligand-mediated collagen blocking blockade.

FIGS. 21A-21B display LAIR-1 binding properties including h11B3 LAIR-1 mAbs binding to cells expressing human LAIR-1 or cynomolgus LAIR-1 as measured by flow cytometry. The h11B3 LAIR-1 mAbs include a variable light chain of SEQ ID NO: 16 and a variable heavy chain of SEQ ID NO: 21, as shown for Anti-LAIR-1 Variant 6 in Table 1 herein. FIG. 21A shows h11B3 binding to a 293T cell line that overexpresses human LAIR-1. Cells were incubated for 30 min on ice with titrated concentrations of h11B3. AF647 conjugated secondary antibody was used at 1:2000 dilution. FIG. 21B shows h11B3 binding to a 293T cell line that overexpresses cynomolgus LAIR-1. Cells were incubated for 30 min on ice with titrated concentrations of h11B3. AF647 conjugated secondary antibody was used at 1:2000 dilution.

FIGS. 22A-22C display binding analysis of h11B3 LAIR-1 mAbs (FIG. 22A) compared to its parent mAb 11B3 (FIG. 22B). The h11B3 LAIR-1 mAbs include a variable light chain of SEQ ID NO: 16 and a variable heavy chain of SEQ ID NO:27, as shown for Anti-LAIR-1 Variant 6 in Table 1 herein. FIG. 22C displays cell binding analysis to MV4-11 cells. LAIR-1-expressing MV4-11 cells were blocked with 2% human serum+2% mouse serum+2% goat serum in FACs Buffer for 10 min on ice, stained with the indicated concentration of AF647-conjugated mAb for 30 min on ice, washed 3× with FACs Buffer, then analyzed by flow cytometry. FACs buffer=phosphate buffered saline+2% fetal bovine serum+1 mM EDTA. FC-G1 BioXcell Ref #BE0096.

FIGS. 23A-23B display h11B3 LAIR-1 mAbs, containing a variable light chain of SEQ ID NO:16 and a variable heavy chain of SEQ ID NO:27, molecular modeling and docking including in silico structural and interaction analysis of LAIR1 and h11B3 predicted amino acid residues of h11B3 binding on LAIR1. LAIR-1 amino acid residues including R28, S30, T37, R50, A77, S80, E81, D114, and Y115 were predicted to interact with h11B3. Analysis was performed using MOE software version 2020.09. FIG. 23B shows LAIR1 with predicted h11B3v6 epitope in yellow.

FIGS. 24A-24C display h11B3 LAIR-1 mAbs, containing a variable light chain of SEQ ID NO: 16 and a variable heavy chain of SEQ ID NO:27, binds LAIR-1 and blocks its functional signaling triggered by collagen-I in UT-140 reporter cells. Seed 1e4 UT140 reporter cells/100 μl in 96-well flat bottom plate. LAIR1 signaling was induced by 10 μg/ml coated collagen treatment. Blocking of LAIR1 signaling by such h11B3 LAIR-1 mAbs was determined by addition of different concentrations of such h11B3 LAIR-1 mAbs in 100 μL per well. Cultures were maintained for 20 hours, and the GFP was read on flow cytometry. FIG. 24A (left) displays reporter cell line UT140 mechanisms. FIG. 24B (middle) displays LAIR-1 binding on UT140. FIG. 24C (right) displays LAIR-1 signaling blocking.

FIGS. 25A-25B display h11B3 LAIR-1 mAbs, containing a variable light chain of SEQ ID NO: 16 and a variable heavy chain of SEQ ID NO:27, blocks collagen-1 signaling. FIG. 25A is a schematic illustration of LAIR-1-TCRζ reporter assay using UT140 NFκB-GFP cell line. FIG. 25B shows where such a human LAIR-1 mAb was competed against Collagen-1 in UT140-LAIR-1-NFAT-GFP reporter cells. The indicated concentration of protein was diluted in phosphate buffer saline and coated to 96-well round-bottom tissue culture plates over-night (O/N) at 4° C. UT140 reporter cells were suspended in complete RPMI media containing 10 μg/mL of the indicated soluble protein, then incubated in coated wells O/N at 37° C. Reporter activation was quantified by flow cytometry. FC-G1 BioXcell Cat #BE0096. Collagen-1 R&D Cat #6220-CL.

FIGS. 26A-26B display h11B3 LAIR-1 mAbs, containing a variable light chain of SEQ ID NO:16 and a variable heavy chain of SEQ ID NO:27, induces AML cell death. Whole blood from AML-diagnosed donors was RBC-depleted using StemCell RBC depletion kit following the manufacturer's instructions. Leukocytes were re-suspended in complete RPMI media containing 3 μg/mL of soluble mAb, then plated in 96-well round-bottom plates for 48 hours at 37° C. Cell death was analyzed by AnnexinV and Fixable Live-Dead staining following the manufacturer's instructions, quantified by flow cytometry. RBC depletion StemCell Ref #18170. Isotype=FC-G1 BioXcell Ref #BE0096. Live-Dead Stain Invitrogen Cat #L34966. AnnexinV-AF647 Biolegend Cat #640943. FIG. 26A displays results for the isotype. FIG. 26B displays results for the h11B3 LAIR-1 mAbs.

FIGS. 27A-27B display h11B3 LAIR-1 mAbs, containing a variable light chain of SEQ ID NO:16 and a variable heavy chain of SEQ ID NO:27, induces AML cell death through antibody dependent cytotoxicity (ADCC). FIG. 27A displays results using a lactate dehydrogenase (LDH) release assay. ADCC was measured using the CytoTox 96® Non-Radioactive Cytotoxicity Assay (Promega) following the manufacturer's instructions. Briefly, AML patient PBMCs were mixed with healthy donor NK cells at a 3:1 Target:Effector ratio in complete RPMI containing the indicated concentration of soluble h11B3 or Fc-G1 isotype control. Mixed cells were incubated for 4 hours at 37 deg C. Test wells were run in quadruplicate. AML PBMCs-Donor #200018616, newly diagnosed/untreated. NK cells-Healthy Donor #83, isolated from PBMCs via StemCell Kit #17955 following manufacturer's instructions. CytoTox 96® Non-Radioactive Cytotoxicity Assay Promega Cat #G1780. % Cytotoxicity quantified by target cell LDH release. % Cytotoxicity=((Test Well LDH Release−Effector Cell Spontaneous Release−Target Cell Spontaneous Release)/(Target Cell Max Release−Target Cell Spontaneous Release))*100. Target Cells=AML PBMCs Donor #200018616; Effector Cells=NK cells isolated from healthy Donor #83 PBMCs. FIG. 27B displays results using an adenylate kinase assay. ADCC was measured using the Toxilight Bioluminescent Cytotoxicity Assay (Lonza)) following the manufacturer's instructions. Briefly, AML patient PBMCs were mixed with healthy donor NK cells at a 3:1 Target:Effector ratio in complete RPMI containing the indicated concentration of soluble h11B3 or Fc-G1 isotype control. Mixed cells were incubated for 4 hours at 37 deg C. Test wells were run in sextuplicate. AML PBMCs-Donor #200003038, newly diagnosed/untreated. NK cells-Healthy Donor #120, isolated from PBMCs via StemCell Kit #17955 following manufacturer's instructions. Toxilight Bioluminescent Cytotoxicity Assay kit Lonza Cat #LT17-217. % Cytotoxicity quantified by target cell adenylate kinase release; ADCC calculated as described above. Target Cells=AML PBMCs Donor #200003038; Effector Cells=NK cells isolated from healthy Donor #120 PBMCs.

FIG. 28 displays h11B3 LAIR-1 mAbs, containing a variable light chain of SEQ ID NO: 16 and a variable heavy chain of SEQ ID NO:27, may promote antibody-dependent cellular phagocytosis (ADCP) of AML cells. LAIR-1 mAb induces ADCP activity in an in-vitro phagocytosis assay utilizing Cell-Trace violet labelled mouse bone marrow macrophages (BMM) cocultured with MV4-11-Luc cells. Mouse BMM was generated with 100 ng/ml M-CSF (Cat #416-ML-500) using seed BMM cells at 1e5/well in 24 well plate. Stain was done with Cell trace violet (Thermofisher). Target MV411 cells were labeled with PKH26 (Sigma #PKH26gl-1kt) according to manufacturer's instructions and cocultured with BMM at ratio of 5:1 in presence of 20 μg/ml h11B3 or control antibody for 16 hours. Phagocytosis was analyzed by flow cytometry.

FIGS. 29A-29C display h11B3 LAIR-1 mAbs, containing a variable light chain of SEQ ID NO: 16 and a variable heavy chain of SEQ ID NO:27, limits Leukemic cell growth in CDX Models. 2e6 luciferase-expressing MV4-11-Luc or THP-1-Luc cells were suspended in sterile PBS and implanted into NSG mice via tail-vein injection. Starting on day 8 post-challenge, mice were treated twice-weekly by intraperitoneal (i.p.) injection of 10 mg/kg of h11B3 LAIR-1 mAbs, containing a variable light chain of SEQ ID NO: 16 and a variable heavy chain of SEQ ID NO:27, or isotype (FIG. 29A). AML cell proliferation was monitored by weekly i.p. injection of D-Luciferin, immediately followed by IVIS bioluminescent imaging and quantification of luminescent signal. Mice NOD.Cg-Prkdcscid Il2rgtm1Wjl/SzJ (NSG) purchased from Jackson Labs strain #005557. Substrate Xenolight RediJect D-Luciferin Perkin Elmore Cat #770504. Imaging Perkin Elmore IVIS Lumina XRMS Series III. FIG. 29B displays MV-4-11 results. FIG. 29C displays THP-1 results.

FIGS. 30A-B display h11B3 LAIR-1 mAbs, containing a variable light chain of SEQ ID NO:16 and a variable heavy chain of SEQ ID NO:27, may promote Antibody-Dependent Cellular Phagocytosis (ADCP) of AML cells. On day 35 of MV411 tumor inoculation, spleen tissue was harvested (FIG. 30A). A portion of spleen was fixed in OCT block. The splenic tissue was mounted on the slides for immunofluorescence staining with hCD45 and mCD45 antibodies and nuclear staining with DAPI dye. Images were captured with NanoString DSP instrument (FIG. 30B). Remaining portion of spleen was subjected to flow cytometry analysis (FIG. 30B). Spleen was processed to produce single cell suspension and percent of dead MV411 cells were enumerated with annexinV and human CD45 antibody staining.

FIGS. 31A-31H shows h11B3 LAIR-1 mAbs, containing a variable light chain of SEQ ID NO:16 and a variable heavy chain of SEQ ID NO:27, reduce tumor burden and restores normal immune cells in AML MV411 using murine model (flow cytometry analysis). NSG-mice were treated with a biweekly dose of 10 mg/kg of such h11B3 LAIR-1 mAbs. Human and mouse CD45 antibodies were used to stain single cells isolated from spleen and bone marrow derived from murine NSG mouse model. FIGS. 31A-31C show a reduction in AML burden and activity in multiple compartments. FIGS. 31D-31F show an induction in AML cell death and specificity to AML cells. FIGS. 31G-31H show protection of normal immune cells and an advantage over chemotherapy.

FIGS. 32A-32C display results of a h11B3 LAIR-1 mAbs, containing a variable light chain of SEQ ID NO:16 and a variable heavy chain of SEQ ID NO:27, dosing Study in MV41-1 model. 2e6 luciferase-expressing MV4-11-Luc cells were suspended in sterile PBS and implanted into NSG mice via tail-vein injection (FIG. 32A). Starting on day 8 post-challenge, mice were treated twice-weekly by intraperitoneal (i.p.) injection of the indicated concentration of h11B3v6 or sterile PBS vehicle. AML cell proliferation was monitored by weekly i.p. injection of D-Luciferin, immediately followed by IVIS bioluminescent imaging and quantification of luminescent signal (FIGS. 32B-32C). Mice NOD.Cg-Prkdcscid Il2rgtm1Wjl/SzJ (NSG) purchased from Jackson Labs strain #005557. Substrate Xenolight RediJect D-Luciferin Perkin Elmore Cat #770504. Imaging Perkin Elmore IVIS Lumina XRMS Series III.

FIGS. 33A-33D display results of a treatment timing study in a MV4-11 model. 2e6 luciferase-expressing MV4-11-Luc cells were suspended in sterile PBS and implanted into NSG mice via tail-vein injection. Starting on the indicated day post-challenge, mice were treated twice-weekly by intraperitoneal (i.p.) injection of 10 mg/kg of h11B3 or sterile PBS vehicle. AML cell proliferation was monitored by weekly i.p. injection of D-Luciferin, immediately followed by IVIS bioluminescent imaging and quantification of luminescent signal. Mice NOD.Cg-Prkdcscid Il2rgtm1Wjl/SzJ (NSG) purchased from Jackson Labs strain #005557. Substrate Xenolight RediJect D-Luciferin Perkin Elmore Cat #770504. Imaging Perkin Elmore IVIS Lumina XRMS Series III.

FIG. 34 displays results of a h11B3 LAIR-1 mAbs, containing a variable light chain of SEQ ID NO: 16 and a variable heavy chain of SEQ ID NO:27, pharmacokinetic study in balb/c mice indicating Tₘₐₓ=8 Hours. Balb/c mice were intraperitoneally injected with 10 mg/kg h11B3. Mice were subsequently bled by cheek-vein puncture at the indicated time points. Blood serum was isolated by centrifugation at 2500 RCF in serum-collection tubes. Blood serum was tested for circulating h11B3 by Universal PK assay. Briefly, h11B3 in mouse serum was captured using a biotin-labeled anti-human IgG inside a nanoliter column of a Gyrolab CD. Alexa Fluor labeled anti-human IgG was used for detection. The quantitative dynamic range was (500-100,000) ng/ml (LLOQ-ULOQ). The standard curve and controls were prepared in pooled Balb/C serum (Biochemed). Standard curve, controls, and samples were diluted 1:10-fold (MRD) in buffer. Samples above the upper limit of quantitation (100,000 ng/mL) were brought within range by diluting them in Rexiip buffer. The laser-induced fluorescence detection technique detected the amount of analyte present in the sample. The standard curve signals were used to interpolate unknown sample concentrations. All samples were analyzed in duplicates and assigned an acceptance criteria (±20% CV and Bias). Gyrolab Evaluator software was used to perform 4PL with Y weighing non-linear regression to interpolate unknown sample concentrations.

FIGS. 35A-35I display a h11B3 LAIR-1 mAbs, containing a variable light chain of SEQ ID NO:16 and a variable heavy chain of SEQ ID NO:27, elicits no depletion, inhibition, or expansion of healthy human primary immune cells in a NSG-SGM3 murine model. NSG-SGM3 mice were purchased from Jackson Laboratory. Mice were engrafted with CD34+ cord blood cells donor #2523. Mice treated weekly beginning 18 weeks after engraftment with anti-human LAIR-1 mAb clone 11B3 in human G1 or G4P format, or control at 100 μg/mouse (5 mg/kg) for 4 weeks. Bleed at weeks 2 (1 day prior to 2nd dose) and 4 (one day prior to 4th dose) for circulating immune cells. Mice were sacrificed 1 week after final dose for analysis of spleen, LN and bone marrow. FIGS. 35A-35B show total body weight and survival of NSG-SGM3 mice reconstituted with human CD34+ cord blood cells and treated 11B3 antibodies. FIGS. 35C-35G show time-course follow-up of the changes in the immune cell numbers in the blood collected from NSG-SGM3 reconstituted with human CD34+ cord blood cells. FIGS. 35H-35I show Analysis of immune cell numbers in the spleen and bone marrow collected from NSG-SGM3 reconstituted with human CD34+ cord blood cells.

### DETAILED DESCRIPTION

The present disclosure can be understood more readily by reference to the following detailed description, examples, drawings, and claims, and their previous and following description. However, before the present compositions and/or methods are disclosed and described, it is to be understood that this disclosure is not limited to the specific compositions and/or methods disclosed unless otherwise specified, as such can, of course, vary. It is also to be understood that the terminology used herein is for the purpose of describing particular aspects only and is not intended to be limiting.

### I. Definitions

Unless defined otherwise, all technical and scientific terms used herein have the same meaning as commonly understood by one of ordinary skill in the art to which this disclosure belongs. Although any compositions and methods similar or equivalent to those described herein can be used in the practice or testing of the present invention. All publications mentioned are incorporated herein by reference in their entirety.

Unless defined otherwise, all composition percentage values used herein are given in terms of weight percentage.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “administration” as it applies to a human, primate, mammal, mammalian subject, animal, veterinary subject, placebo subject, research subject, experimental subject, cell, tissue, organ, or biological fluid, refers to contact of an exogenous ligand, reagent, placebo, small molecule, pharmaceutical agent, therapeutic agent, diagnostic agent, or composition to the subject, cell, tissue, organ, or biological fluid, and the like. “Administration” can refer to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

An “agonist,” as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a mutein or derivative of GM-CSF, a peptide mimetic of GM-CSF, a small molecule that mimics the biological function of GM-CSF, or an antibody that stimulates GM-CSF receptor.

As used herein, an “analog” or “derivative” with reference to a peptide, polypeptide or protein refers to another peptide, polypeptide or protein that possesses a similar or identical function as the original peptide, polypeptide or protein, but does not necessarily comprise a similar or identical amino acid sequence or structure of the original peptide, polypeptide or protein. An analog preferably satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the original amino acid sequence (b) a proteinaceous agent encoded by a nucleotide sequence that hybridizes under stringent conditions to a nucleotide sequence encoding the original amino acid sequence; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the original amino acid sequence.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, “antibody” refers to a peptide or polypeptide derived from, modeled after, or substantially encoded by an immunoglobulin gene or immunoglobulin genes, or fragments thereof, capable of specifically binding an antigen or epitope (Wilson, J. Immunol. Methods, 1994; Yarmush, J. Biochem. Biophys, 1992). The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward, et al., Nature, 1989), which consists of a VH domain; and (vi) an isolated complementarity determining region (CDR). Single chain antibodies are also included by reference in the term “antibody.”

As used herein, “antigen presenting cells” (APCs) are cells of the immune system used for presenting antigen to T cells. APCs include dendritic cells, monocytes, macrophages, marginal zone Kupffer cells, microglia, Langerhans cells, T cells, and B cells. Dendritic cells occur in at least two lineages. The first lineage encompasses pre-DC1, myeloid DC1, and mature DC1. The second lineage encompasses CD34⁺CD45RA⁻ early progenitor multipotent cells, CD34⁺CD45RA⁺ cells, CD34⁺CD45RA⁺CD4⁺ IL-3Rα⁺ pro-DC2 cells, CD4⁺CD11c⁻ plasmacytoid pre-DC2 cells, lymphoid human DC2 plasmacytoid-derived DC2s, and mature DC2s.

As used herein, the term “cancer” refers to a neoplasm or tumor resulting from abnormal uncontrolled growth of cells. As used herein, cancer explicitly includes, sarcoma, carcinoma, leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or web-like matrices in a three-dimensional basement membrane or extracellular matrix preparation. Non-cancer cells do not form colonies in soft agar and form distinct sphere-like structures in three-dimensional basement membrane or extracellular matrix preparations.

As used herein, the term “chimeric receptor” is defined as a cell-surface receptor comprising an extracellular ligand binding domain, a transmembrane domain and a cytoplasmic co-stimulatory signaling domain in a combination that is not naturally found together on a single protein. This particularly includes receptors wherein the extracellular domain and the cytoplasmic domain are not naturally found together on a single receptor protein. Further, the chimeric receptor is different from the TCR expressed in the native T cell lymphocyte.

As used herein, the “co-stimulatory” signals encompass positive co-stimulatory signals (e.g., signals that result in enhancing an activity) and negative co-stimulatory signals (e.g., signals that result in inhibiting an activity).

The term “derivative” refers to an antibody or antigen-binding fragment thereof that immunospecifically binds to the same target of a parent or reference antibody but which differs in amino acid sequence from the parent or reference antibody or antigen binding fragment thereof by including one, two, three, four, five or more amino acid substitutions, additions, deletions or modifications relative to the parent or reference antibody or antigen binding fragment thereof. In some embodiments such derivatives will have substantially the same immunospecificity and/or characteristics, or the same immunospecificity and characteristics as the parent or reference antibody or antigen binding fragment thereof. The amino acid substitutions or additions of such derivatives can include naturally occurring (i.e., DNA-encoded) or non-naturally occurring amino acid residues. The term “derivative” encompasses, for example, chimeric or humanized variants, as well as variants having altered CH1, hinge, CH2, CH3 or CH4 regions, so as to form, for example antibodies, etc., having variant Fc regions that exhibit enhanced or impaired effector or binding characteristics.

As used herein, “effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition.

As used herein, “epitope” refers to an antigenic determinant capable of specific binding to an antibody. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

As used herein, “extracellular fluid” encompasses serum, plasma, blood, interstitial fluid, cerebrospinal fluid, secreted fluids, lymph, bile, sweat, fecal matter, and urine. An “extracellular fluid” can comprise a colloid or a suspension, such as whole blood or coagulated blood.”

As used herein, “fragments” in the context of polypeptides include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a larger polypeptide.

As used herein, the term “humanized antibody” refers to an immunoglobulin including a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they should be substantially identical to human immunoglobulin constant regions, i.e., at least about 85-99%, or about 95% or more identical. Hence, all parts of a humanized immunoglobulin, except possibly the CDR's, are substantially identical to corresponding parts of natural human immunoglobulin sequences. A humanized antibody is an antibody including a humanized light chain and a humanized heavy chain immunoglobulin. For example, a humanized antibody would not encompass a typical chimeric antibody, because, e.g., the entire variable region of a chimeric antibody is non-human.

As used herein, an “immune cell” refers to any cell from the hemopoietic origin including, but not limited to, T cells, B cells, NK cell, monocytes, dendritic cells, and macrophages.

An “immunogenic agent” or “immunogen” is capable of inducing an immunological response against itself on administration to a mammal, optionally in conjunction with an adjuvant.

As used herein, the terms “immunologic,” “immunological” or “immune” response is the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, “inflammatory molecules” refer to molecules that result in inflammatory responses including, but not limited to, cytokines and metalloproteases such as including, but not limited to, IL-1β, TNF-α, TGF-beta, IFN-γ, IL-18, IL-17, IL-6, IL-23, IL-22, IL-21, and MMPs.

As used herein, “ligand” refers to a small molecule, peptide, polypeptide, or membrane associated or membrane-bound molecule, that is an agonist or antagonist of a receptor. “Ligand” also encompasses a binding agent that is not an agonist or antagonist, and has no agonist or antagonist properties. By convention, where a ligand is membrane-bound on a first cell, the receptor usually occurs on a second cell. The second cell may have the same identity (the same name), or it may have a different identity (a different name), as the first cell. A ligand or receptor may be entirely intracellular, that is, it may reside in the cytosol, nucleus, or in some other intracellular compartment. The ligand or receptor may change its location, such as from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

As used herein the term “modulate” relates to a capacity to alter an effect, result, or activity (e.g., signal transduction). Such modulation can be agonistic or antagonistic. Antagonistic modulation can be partial (i.e., attenuating, but not abolishing) or it can completely abolish such activity (e.g., neutralizing). Modulation can include internalization of a receptor following binding of an antibody or a reduction in expression of a receptor on the target cell. Agonistic modulation can enhance or otherwise increase or enhance an activity (e.g., signal transduction). In a still further embodiment, such modulation can alter the nature of the interaction between a ligand and its cognate receptor so as to alter the nature of the elicited signal transduction. For example, the molecules can, by binding to the ligand or receptor, alter the ability of such molecules to bind to other ligands or receptors and thereby alter their overall activity. In some embodiments, such modulation will provide at least a 10% change in a measurable immune system activity, at least a 50% change in such activity, or at least a 2-fold, 5-fold, 10-fold, or at least a 100-fold change in such activity.

As used herein, the terms “percent sequence identity” and “% sequence identity” refer to the percentage of sequence similarity found by a comparison or alignment of two or more amino acid or nucleic acid sequences. Percent identity can be determined by a direct comparison of the sequence information between two molecules by aligning the sequences, counting the exact number of matches between the two aligned sequences, dividing by the length of the shorter sequence, and multiplying the result by 100. An algorithm for calculating percent identity is the Smith-Waterman homology search algorithm (see, e.g., Kann and Goldstein (2002) Proteins 48:367-376; Arslan, et al. (2001) Bioinformatics 17:327-337).

As used herein, “peptide” refers to a short sequence of amino acids, where the amino acids are connected to each other by peptide bonds. A peptide may occur free or bound to another moiety, such as a macromolecule, lipid, oligo- or polysaccharide, and/or a polypeptide. Where a peptide is incorporated into a polypeptide chain, the term “peptide” may still be used to refer specifically to the short sequence of amino acids. A “peptide” may be connected to another moiety by way of a peptide bond or some other type of linkage. A peptide is at least two amino acids in length, wherein the maximal length is a function of custom or context.

As used herein, a “pharmaceutically acceptable excipient” or “diagnostically acceptable excipient” includes but is not limited to, sterile distilled water, saline, phosphate buffered solutions, amino acid based buffers, or bicarbonate buffered solutions. An excipient selected and the amount of excipient used will depend upon the mode of administration. Administration comprises an injection, infusion, or a combination thereof.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “protein” generally refers to the sequence of amino acids comprising a polypeptide chain. Protein may also refer to a three-dimensional structure of the polypeptide. “Denatured protein” refers to a partially denatured polypeptide, having some residual three-dimensional structure or, alternatively, to an essentially random three dimensional structure, as is the case in a totally denatured protein. Polypeptide variants can be produced by glycosylation, phosphorylation, sulfation, disulfide bond formation, deamidation, isomerization, cleaving points in signal or leader sequence processing, covalent and non-covalently bound cofactors, oxidized variants, and the like.

As used herein, “recombinant” when used with reference to a nucleic acid, cell, animal, virus, plasmid, vector, or the like, indicates modification by the introduction of an exogenous, non-native nucleic acid, alteration of a native nucleic acid, or by derivation in whole or in part from a recombinant nucleic acid, cell, virus, plasmid, or vector. Recombinant protein refers to a produced or secreted protein derived from a recombinant nucleic acid, virus, plasmid, vector, or the like.

As used herein, “sample” refers to a sample from a human, animal, placebo, or research sample, such as a cell, tissue, organ, fluid, gas, aerosol, slurry, colloid, or coagulated material. The “sample” may be tested in vivo, (i.e. without removal from the human or animal), or it may be tested in vitro. The sample may be tested after processing, such as by histological methods. “Sample” also refers to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

“Specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

As used herein, the term “subject” refers to a human or non-human organism. Thus, the methods and compositions described herein are applicable to both human and veterinary disease. In certain embodiments, subjects are “patients,” such as living humans that are receiving medical care for a disease or condition. This includes persons with no defined illness who are being investigated for signs of pathology.

The term “substantially,” as used in the context of binding or exhibited effect, is intended to denote that the observed effect is physiologically or therapeutically relevant. Thus, for example, a molecule is able to substantially block an activity of a ligand or receptor if the extent of blockage is physiologically or therapeutically relevant (for example if such extent is greater than 60% complete, greater than 70% complete, greater than 75% complete, greater than 80% complete, greater than 85% complete, greater than 90% complete, greater than 95% complete, or greater than 97% complete). Similarly, a molecule is said to have substantially the same immunospecificity and/or characteristic as another molecule, if such immunospecificities and characteristics are greater than 60% identical, greater than 70% identical, greater than 75% identical, greater than 80% identical, greater than 85% identical, greater than 90% identical, greater than 95% identical, or greater than 97% identical).

As used herein, the term “therapeutically effective amount” is defined as an amount of a reagent or pharmaceutical composition that is sufficient to induce a desired immune response specific for encoded heterologous antigens to show a patient benefit (e.g. to cause a decrease, prevention, or amelioration of the symptoms of the condition being treated). When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual (U.S. Pat. No. 5,888,530).

As used herein, “treatment” or “treating” (with respect to a condition or a disease) is an approach for obtaining beneficial or desired results including and preferably clinical results. For purposes of this disclosure, beneficial or desired results with respect to a disease include, but are not limited to, one or more of improving a condition associated with a disease, curing a disease, lessening severity of a disease, delaying progression of a disease, alleviating one or more symptoms associated with a disease, increasing the quality of life of one suffering from a disease, and/or prolonging survival. Likewise, for purposes of this disclosure, beneficial or desired results with respect to a condition include, but are not limited to, one or more of improving a condition, curing a condition, lessening severity of a condition, delaying progression of a condition, alleviating one or more symptoms associated with a condition, increasing the quality of life of one suffering from a condition, and/or prolonging survival.

As used herein, “tumor microenvironment” or “TME” refers to the normal cells, molecules, fibroblasts, immune cells, and blood vessels that surround and feed a tumor cell. The tumor microenvironment also includes proteins produced by all of the cells present in the tumor that support the growth of the cancer cells, including ECM.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

### II. LAIR-1

Leukocyte Associated Immunoglobulin Like Receptor 1 (LAIR-1) is a target highly expressed on leukemic stem and blast cells and mediates survival of these cells. The present disclosure relates to LAIR-1 antibodies that specifically kill leukemic stem and blast cells while preserving healthy hematopoietic stem cells.

LAIR-1 is the only know collagen receptor with inhibitory signaling capacity. This further implicates a critical role of LAIR-1 mediated inhibitory signaling in AML cells (Ricard-Blum, The Collagen Family Review, 2011; Bo An et al., Collagen Interactions: Drug Design and Delivery, 2015). Collagen and the ECM regulate a wide variety of cell membrane dynamics (reviewed in Hu et al., 2022), and ECM ligands can impact signaling dynamics and subsequent cell fate of leukemic cells, particularly in the bone marrow (Shin et al, 2016; Galán-Díez et al, 2018; Zanetti and Krause, 2020). These differential signaling dynamics present a possible reason for the discrepancy between reports showing LAIR-1 activation of cell survival mechanisms, such as CAMK1/CREB (Kang, Lu et al., 2015; Kang, Kim et al., 2016), and reports showing LAIR-1-mediated growth arrest and cell death (Poggi et al., 2000; Zhocchi et al., 2001).

Sequences for LAIR-1 are provided. In some embodiments, the leading methionine amino acid is cleaved in the post-translation form of the protein.

### A. Human LAIR-1

Sequences for human LAIR-1 are known in the art. For example, a consensus sequence for LAIR-1a (isoform 1) is:

MSPHPTALLGLVLCLAQTIHTQEEDLPRPSISAEPGTVIPLGSHVTFVC
RGPVGVQTFRLERESRSTYNDTEDVSQASPSESEARFRIDSVSEGNAGP
YRCIYYKPPKWSEQSDYLELLVKETSGGPDSPDTEPGSSAGPTQRPSDN
SHNEHAPASQGLKAEHLYILIGVSVVFLFCLLLLVLFCLHRQNQIKQGP
PRSKDEEQKPQQRPDLAVDVLERTADKATVNGLPEKDRETDTSALAAGS
SQEVTYAQLDHWALTQRTARAVSPQSTKPMAESITYAAVARH

(SEQ ID NO:1, UniProtKB-Q6GTX8 (LAIR1_HUMAN)), where amino acids 1-21 are a signal sequence, amino acids 22-165 (underlined) are an extracellular domain, amino acids 166-186 are a transmembrane domain, and amino acids 187-287 are a cytoplasmic domain. Amino acids 29-117 form an Ig-like C2-domain. Amino acids 249-254 and 279-284 form ITIM motif 1 and 2, respectively. LAIR-1b (also known as isoform 2) is missing amino acids 122-138 relative to SEQ ID NO:1. LAIR-1c (also known as isoform 3) is missing amino acids 23-23 and 122-138 relative to SEQ ID NO:1. LAIR-1d (also known as isoform 4) is missing amino acids 210-287 relative to SEQ ID NO:1.

As introduced above, an extracellular domain for human LAIR-1 can be:

QEEDLPRPSISAEPGTVIPLGSHVTFVCRGPVGVQTFRLERESRSTYND
TEDVSQASPSESEARFRIDSVSEGNAGPYRCIYYKPPKWSEQSDYLELL
VKETSGGPDSPDTEPGSSAGPTQRPSDNSHNEHAPASQGLKAEHLY

(SEQ ID NO:2), or a fragment thereof, for example, the Ig-like C2-domain (underlined amino acids 8-96 of SEQ ID NO:2), or the region framed by the cysteines that form the disulfide bond between amino acids 49-101 of SEQ ID NO: 1 (amino acids 28-80 of SEQ ID NO:2, illustrated in italics).

Known variants and mutants of LAIR-1 include E63D, Y251F, and Y251F, relative to SEQ ID NO:1. Evidence shows that Y215F reduced tyrosine phosphorylation and loss of binding to PTPN6 and CSK as well as complete loss of inhibitory activity, as well as loss of phosphorylation and of inhibition of calcium mobilization when associated with F-281 (Xu, et al., J. Biol. Chem. 275:17440-17446 (2000), Verbrugge, et al., Int. Immunol., 15:1349-1358 (2003), Verbrugge, et al., Eur. J. Immunol., 36:190-198 (2006)). Y281F shows reduced tyrosine phosphorylation and loss of binding to PTPN6, and partial inhibition of cytotoxic activity.

Meyaard, 2008, J. Leukoc. Biol. 83:799-803 indicates that LAIR-1 is broadly expressed on human immune cells. An examination of actual flow cytometry expression data in research papers shows that LAIR-1 is much more highly expressed on myeloid lineage cells such as monocytes, macrophages and dendritic cells, than on T cells and NK cells (Meyaard et al., 1997, Immunity 7:283-290). However, B cells differentially express high levels of LAIR-1 during differentiation (van der Vuurst de Vries et al., 1999, Eur. J. Immunol. 29:3160-3167). LAIR-1 has also been found to be expressed on acute myeloid leukemia cells, acute lymphoblastic leukemia cells and chronic lymphocytic leukemia cells (van der Vuurst de Vries et al., 1999, Eur. J. Immunol. 29:3160-3167; Poggi et al., 2000, Eur. J. Immunol. 30:2751-2758; Zocchi et al., 2001, Eur. J. Immunol. 31:3667-3675; Perbellini et al., 2014, Haematologica, 99:881-887; (Kang et al., 2015, Nat. Cell Biol. 17:665-677). Finally, LAIR-1 was shown be expressed on several human tumor cell lines (Meyaard et al., 1997, Immunity 7:283-290; Cao et al., 2015, Biochem. Biophys, Res. Commun. 458:399-404; (Kang et al., 2015, Nat. Cell Biol. 17:665-677).

In humans and mice, LAIR-1 binds several types of collagen with high affinity (Meyaard, 2008, J. Leukoc. Biol. 83:799-803 and Meyaard, 2010, Immunol. Lett. 128:26-28). In humans, LAIR-1 has also been shown to bind the complement component Clq (Son et al., 2012, Proc. Natl. Acad. Sci. USA 109: E3160-3167) and the collagenous C-type lectin, surfactant protein-D (SP-D), a collagenous carbohydrate binding glycoprotein (collectin) that plays important roles in the lung's innate immune response to microbial and antigenic challenge (Olde Nordkamp et al., 2014, J. Leukoc. Biol. 96:105-111). The ability of murine LAIR-1 to bind C1q and SP-D has not been examined.

### B. Mouse LAIR-1

Sequences for mouse LAIR-1 (mLAIR-1) are known in the art. For example, a consensus sequence for mLAIR-1a (isoform 1) is:

MSLHPVILLVLVLCLGWKINTQEGSLPDITIFPNSSLMISQGTFVTVVC
SYSDKHDLYNMVRLEKDGSTFMEKSTEPYKTEDEFEIGPVNETITGHYS
CIYSKGITWSERSKTLELKVIKENVIQTPAPGPTSDTSWLKTYSIYIFT
VVSVIFLLCLSALLFCFLRHRQKKQGLPNNKRQQQRPEERLNLATNGLE
MTPDIVADDRLPEDRWTETWTPVAGDLQEVTYIQLDHHSLTQRAVGAVT
SQSTDMAESSTYAAIIRH

(SEQ ID NO:3, UniProtKB-Q8BG84 (LAIR1_MOUSE)), where amino acids 1-21 are a signal sequence, amino acids 22-144 (underlined) are an extracellular domain, amino acids 145-165 are a transmembrane domain, and amino acids 166-263 are a cytoplasmic domain. Amino acids 27-115 form an Ig-like C2-domain. Amino acids 226-231 and 255-260 form ITIM motif 1 and 2, respectively. mLAIR-1b (also known as isoform 2) is missing amino acids 124-133 relative to SEQ ID NO:3. Isoform 3 has amino acids 25-56 [SLPDITIFPNSSLMISQGTFVTVVCSYSDKHD (SEQ ID NO:4) of SEQ ID NO: 3)] replaced with ELCLWFLLYPWATLELIMCTWDAWKETLEYFL (SEQ ID NO: 5) and is missing amino acids 57-263 relative to SEQ ID NO:3. mLAIR-1d (also known as isoform 5) is missing amino acids 24-172 relative to SEQ ID NO:3. mLAIR-1e (also known as isoform 6) is missing amino acids 134-172.

As introduced above, an extracellular domain for murine LAIR-1 can be

QEGSLPDITIFPNSSLMISQGTFVTVVCSYSDKHDLYNMVRLEKDGSTF
MEKSTEPYKTEDEFEIGPVNETITGHYSCIYSKGITWSERSKTLELKVI
KENVIQTPAPGPTSDTSWLKTYSIY

(SEQ ID NO:6), or a fragment thereof, for example, the Ig-like C2-domain (underlined amino acids 6-94 of SEQ ID NO:6), or the region framed by the cysteines that form the disulfide bond between amino acids 49-99 of SEQ ID NO:3 (amino acids 28-78 of SEQ ID NO:6, illustrated in italics). An exemplary alignment of the human and mouse extracellular domains is shown below:

Known variants and mutants of LAIR-1 include IYI→MYM at amino acid positions 143-145, V149G, L154P, and H263R relative to SEQ ID NO:3.

Meyaard (2008, J. Leukoc. Biol. 83:799-803) indicates broad expression of LAIR-1 on mouse immune cells, with one major difference being that LAIR-1 appears negative on B cells, as opposed to being highly expressed on subsets of human B cells. As with the human expression pattern, when examining the actual flow cytometry data of LAIR-1 expression, it is found that once again, LAIR-1 is highly expressed on monocytes, macrophages and DCs, while T cells, NK cells and Gr-1+ cells express LAIR-1 at relatively lower levels (Lebbink et al., 2007 Int. Immunol. 19:1011-1019; Tang et al., 2012, J. Immunol. 188:548-558).

Tang et al. (2012, J. Immunol. 188:548-558) investigated the phenotype of LAIR-1 deficient mice. KO mice are healthy and fertile, and display indications of altered immune function, but without gross autoimmunity or inflammation that is observed in CTLA-4 KO mice. LAIR-1 KO mice have increased numbers of dendritic cells, splenic B cells and regulatory T cells, as well as a higher frequency of activated and memory T cells, suggesting enhanced T cell reactivity. However, there was no difference in EAE and colitis disease models in LAIR-1 WT and KO mice. These disease models may not have been optimal for investigating LAIR-1 KO phenotype, and in vitro functional studies of LAIR-1 deficient immune cell subsets were not performed. It is also speculated that LAIR-1 KO mice may not be indicative of the role of LAIR-1 in humans due to differential expression and the presence of soluble LAIR-2 in humans. Differences between LAIR-1 genetic pathways in murine and human internal organs are discussed in Sun, et al., Gene, 552:14-145 (2014), and can be accounted for when designing and evaluating experiments utilizing a mouse model.

### III. Immunomodulatory Agents

The present disclosure relates to immunomodulatory agents including agonists of LAIR-1. As non-limiting examples, such immunomodulatory agents include anti-LAIR-1 antibodies, as described further below. An agonist of LAIR-1 typically induces, potentiates, or activates LAIR-1 negative signaling. The compositions and methods can be used to modulate LAIR-1 negative signaling on, for example, myeloid cells including antigen-presenting cells (e.g., monocyte, macrophage, or dendritic cell), T cells, Natural Killer (NK) cells, or a combination thereof. In some embodiments, the compositions are specifically targeted one or more cells types. Exemplary molecules that can be an agonist of LAIR-1 are discussed in more detail herein.

### A. Antibodies

The immunomodulatory agent can be an antibody. Suitable antibodies are described below. The sequences can be used, as discussed in more detail below, by one of skill in the art to prepare an antibody or antigen binding fragment thereof specific for LAIR-1. The antibody, or antigen binding fragment therefore, can be an agonist of LAIR-1.

The activity of an antibody or antigen binding fragment thereof that is specific for LAIR-1, can be determined using functional assays that are known in the art, and include the assays discussed below. Typically, the assays include determining if the antibody or antigen binding fragment thereof increases (i.e., agonist) signaling through LAIR-1. In some embodiments the assay includes determining if the antibody or antigen binding fragment thereof decreases (i.e., agonist) an immune response negatively regulated by LAIR-1.

In some embodiments, the disclosed antibodies and antigen binding fragments thereof immunospecifically bind to LAIR-1. In some embodiments, the antibody binds to an extracellular domain of LAIR-1.

For example, molecules are provided that can immunospecifically bind to LAIR-1:
(I) arrayed on the surface of a cell (especially a live cell);
(II) arrayed on the surface of a cell (especially a live cell) at an endogenous concentration;
(III) arrayed on the surface of a live cell, and modulates binding between LAIR-1 and a ligand thereof;
(IV) arrayed on the surface of a live cell, and reduces or inhibits immune suppression by LAIR-1;
(V) arrayed on the surface of a live cell, and induces or enhances immune suppression by LAIR-1;
(VI) arrayed on the surface of a live cell, wherein the cell is a myeloid cell including antigen-presenting cells (e.g., monocyte, macrophage, or dendritic cell), a T cell, a Natural Killer (NK) cell, or a combination thereof;
(VII) combinations of I-IV and VI;
(VIII) combinations of I-III and V-IV; and
(IX) arrayed on the surface of a live myeloid or lymphoid derived cancer cells (AML or ALL), and enhances apoptosis and differentiation resulting in reduced self-renewal of cancer stem cells.

In some embodiments, the molecules are capable of inducing antibody dependent cell cytotoxicity (ADCC), complement dependent cytotoxicity (CDC) or cellular apoptosis through other mechanisms, of LAIR-1 expressing cell.

To prepare an antibody or antigen binding fragment thereof that specifically binds to LAIR-1, purified proteins, polypeptides, fragments, fusions, or epitopes to LAIR-1, or polypeptides expressed from nucleic acid sequences thereof, can be used. The antibodies or antigen binding fragments thereof can be prepared using any suitable methods known in the art such as those discussed in more detail below.

i. Compositions of Humanized LAIR-1 Antibodies

Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also contain residues that are found neither in the recipient antibody nor in the imported CDR or framework sequences. In general, the humanized antibody will contain substantially all of at least one, and typically two, variable domains, in which all or substantially all, of the CDR regions correspond to those of a non-human immunoglobulin and all or substantially all of the FR regions are those of a human immunoglobulin consensus sequence. The humanized antibody optimally also will contain at least a portion of an immunoglobulin constant region (Fc), typically that of a human immunoglobulin.

Methods for humanizing non-human antibodies are well known in the art. See for example, Jones, P. T., et al. (1986). Replacing the complementarity-determining regions in a human antibody with those from a mouse. Nature 321, 522-525. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a nonhuman antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

The antibody can be bound to a substrate or labeled with a detectable moiety or both bound and labeled. The detectable moieties contemplated with the present compositions include fluorescent, enzymatic and radioactive markers.

The present disclosure relates to compositions comprising monoclonal antibodies for targeting LAIR-1. In leukemia, a significant unmet need remains for a therapeutic that selectively eradicates leukemia stem cells (LSCs) and leukemic blasts but spares normal hematopoietic stem cells. LAIR-1 is an immune receptor previously identified as a potential target for therapeutic intervention in AML.

The present disclosure displays that augmentation of LAIR-1 signaling in the physiological setting of natural collagens is critical to its anti-cancer function and that LAIR-1 signaling facilitated the induction of AML cell death. This function appears particularly in the context of collagen. Disclosed herein is a novel function of LAIR-1 in AML and a strategy for treating AML by targeting LAIR-1 with an agonist antibody.

The present disclosure relates to an agonist LAIR-1 monoclonal antibody, that effectively depletes LSCs and blasts both ex vivo and in vivo while sparing healthy cells. LAIR-1 mAb exhibits increased promotion of the death of LSCs and AML blasts both in vivo and ex vivo in the context of LAIR-1 clustering. LAIR-1 mAb exhibits minimal effects on healthy HSCs or immune cells.

ii. Exemplary Humanized Antibodies

One embodiment provides a humanized monoclonal antibody (mAb), LAIR-1 mAb. LAIR-1 mAb is produced by a hybridoma selected from the group consisting of 10D6 and 11B3.

Another embodiment provides a humanized monoclonal LAIR-1 antibody having at least one light chain or at least one heavy chain of the antibody produced by one or more of the hybridomas selected from the group consisting of 10D6 and 11B3.

Another embodiment provides a humanized monoclonal LAIR-1 antibody having a variable light chain having at least 50, 60, 70, 80, 85, 90, 95, 96, 97, 98, 99, or 100% sequence identity to a variable light chain having an amino acid sequence according to SEQ ID NO: 7, 8, 12, 14, 15, 16, or 17.

Another embodiment provides a humanized monoclonal LAIR-1 antibody having a variable heavy chain having at least 50, 60, 70, 80, 85, 90, 95, 96, 97, 98, 99, or 100% sequence identity to a variable heavy chain having an amino acid sequence according to SEQ ID NO: 9, 18, 21, 22, 23, 24, 25, 26, 27, 28, 29, 30, 31, 32, 33, 34, 35, 36, 37, 38, 39, 40, 41, 42, 43, or 44.

Another embodiment provides a humanized monoclonal LAIR-1 antibody having a variable light chain having at least 50, 60, 70, 80, 85, 90, 95, 96, 97, 98, 99, or 100% sequence identity to a variable light chain having an amino acid sequence according to SEQ ID NO: 7, 8, 12, 14, 15, 16, or 17, and a variable heavy chain having at least 50, 60, 70, 80, 85, 90, 95, 96, 97, 98, 99, or 100% sequence identity to an amino acid sequence according to SEQ ID NO: 9, 18, 21, 22, 23, 24, 25, 26, 27, 28, 29, 30, 31, 32, 33, 34, 35, 36, 37, 38, 39, 40, 41, 42, 43, or 44.

Another embodiment provides a humanized monoclonal LAIR-1 antibody having a heavy chain with an amino acid sequence at least 50, 60, 70, 80, 85, 90, 95, 96, 97, 98, 99, or 100% sequence identity to the amino acid sequence set forth in SEQ ID NO: 10, 11, 19, or 20 and/or a light chain with an amino acid sequence at least 50, 60, 70, 80, 85, 90, 95, 96, 97, 98, 99, or 100% sequence identity to the amino acid sequence set forth in SEQ ID NO: 8 or 13.

### IV. Methods of Use of Humanized LAIR-1

The present disclosure relates to methods of modulating the ability to specifically deplete cancer cells, excluding normal cells, as a key component of an effective cancer therapy. The present disclosure provides a novel understanding of LAIR-1 biology and a novel method for therapeutic intervention of AML through targeting of LAIR-1 with an agonist mAb including LAIR-1 mAb. Methods and compositions disclosed herein can be used to treat patients with AML, relapsed refractory Chronic Myelo-monocytic Leukemia (CMML) and high-risk myelodysplastic syndrome (MDS).

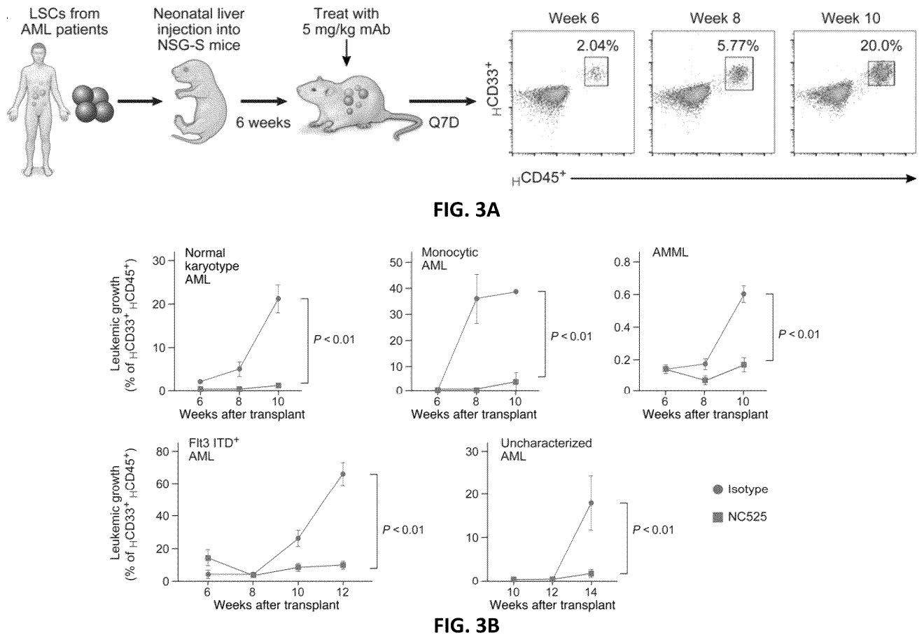

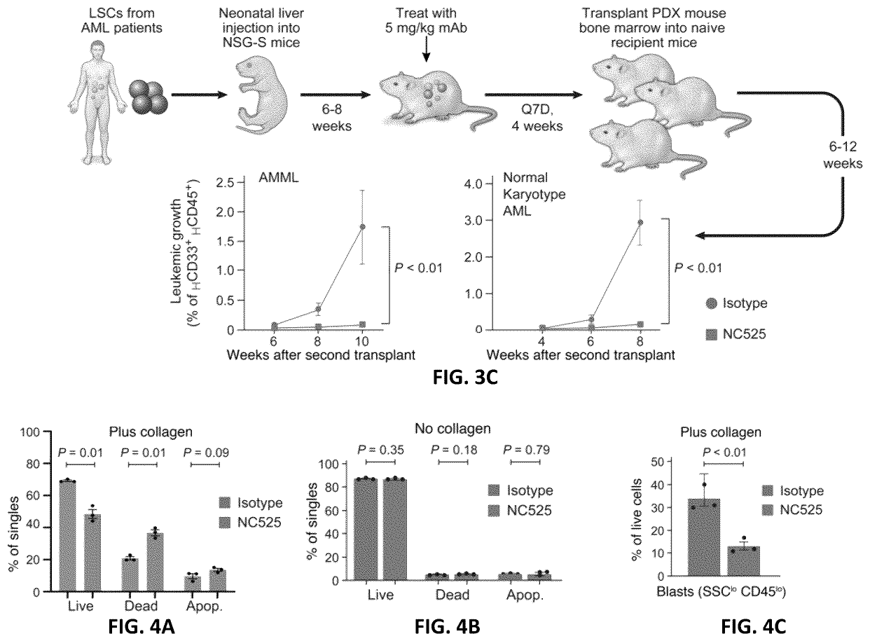

The present disclosure relates to methods of use of an agonist LAIR-1 monoclonal antibody, LAIR-1 mAb, that targets LAIR-1+ leukemic cells to induce strong SHP-1 signaling. This is a novel and effective approach for the inhibition and depleting of AML blast and LSC progenitors. As disclosed herein, engagement of LAIR-1 by the agonist mAb induces cell death in leukemic blasts and LSCs to sustain therapeutic efficacy and reduce relapse. This is demonstrated in a secondary transplant PDX model (FIG. 3C).

LAIR-1 mAb additionally inhibits AML in two CDX models and in more difficult to treat PDX models of disease in a robust manner, as shown in FIGS. 3A and 3B. The CDX models demonstrates systemic inhibition of AML growth that is dependent on LAIR-1 expression level and agnostic to adaptive immunity. The PDX models agree with in vitro findings that demonstrate LAIR-1 mAb mediated inhibition of LSC colony growth.

The unique activity of LAIR-1 mAb works in venetoclax/azacitidine (VEN/AZA) sub-responsive AML. Combinatorial treatment with VEN/AZA elicits an additive or synergistic therapeutic effect, opening the opportunity for several therapeutic options for LAIR-1 mAb treatment of patients with unmet needs.

The present disclosure provides a significant downregulation of B-Cell Lymphoma-Extra Large (BCL-XL) during LAIR-1 mAb treatment. As a non-limiting example, patients become resistant to VEN/AZA due to upregulation of BCL-XL despite the effective suppression of B-Cell Lymphoma 2 (BCL-2) and Myeloid Leukemia-1 (MCL-1) by VEN and AZA, respectively. A combination of VEN/AZA with LAIR-1 mAb effectively suppresses BCL-2/MCL-1 through BCL-XL to induce synergistic depleting of AML cells.

LAIR-1 mAb activity may be enhanced by antibody-dependent cell cytotoxicity (ADCC) and antibody-dependent cellular phagocytosis (ADCP). However, while it is believed the activity of LAIR-1 mAb is exerted by LAIR-1 mAb crosslinking through interaction with Fc receptors, the lack of activity of LAIR-1 mAb on healthy LAIR-1+ immune cells indicates that ADCC and ADCP are not primary mechanisms of action for LAIR-1 mAb. Beyond potential interactions in trans, intracellular signaling through Fc receptors has been shown to impact cell survival in a number of contexts, including leukemia (Parting et al, 2020). The interaction of the LAIR-1 mAb IgG domain with FcγRs could not only promote downstream signal transduction, but may also stabilize a focal synapse with LAIR-1 and ECM components in cis to promote crosslinking and overcome the signaling threshold necessary to initiate apoptosis.

Together, the study presented here points to LAIR-1 as a central inhibitory receptor regulating signaling in response to collagen and the ECM. This concept will be the major focus of future studies. A better understanding of LAIR-1 localization and communication with other collagen and ECM interacting proteins may allow for tuning of signaling patterns in both homeostatic and non-homeostatic cellular environments. Importantly, understanding the network that dictates cell fate will lead to novel target discovery, inform on ideal interventions for AML and other diseases, and generate optimized therapeutic combinatorial strategies.

The present disclosure additionally relates to methods of use of LAIR-1 mAb in additional leukemia models. As non-limiting examples, such models include but are not limited to VEN/AZA non-responder models from M5 AML patients, and further combinations known in the art.

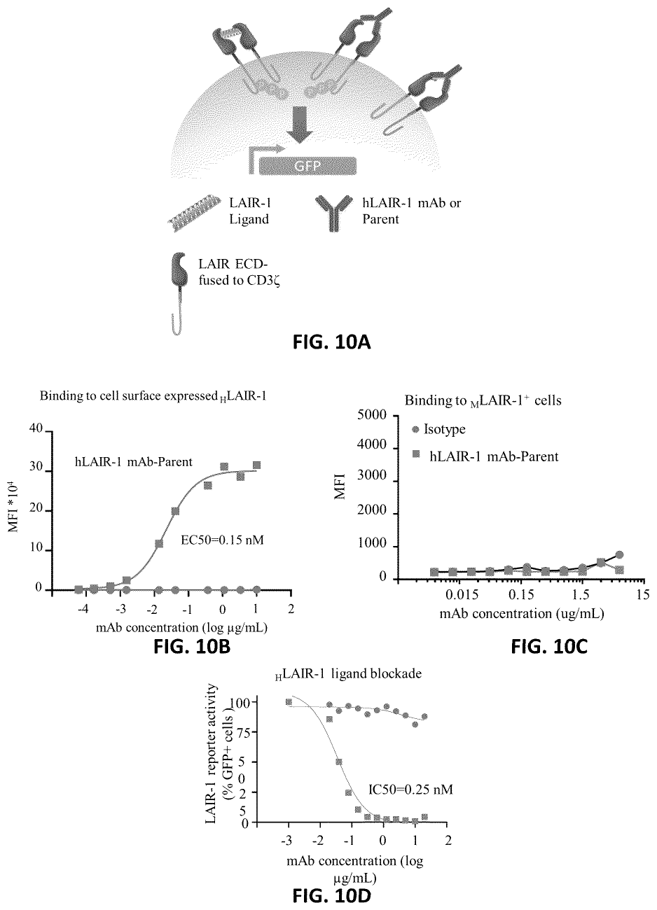

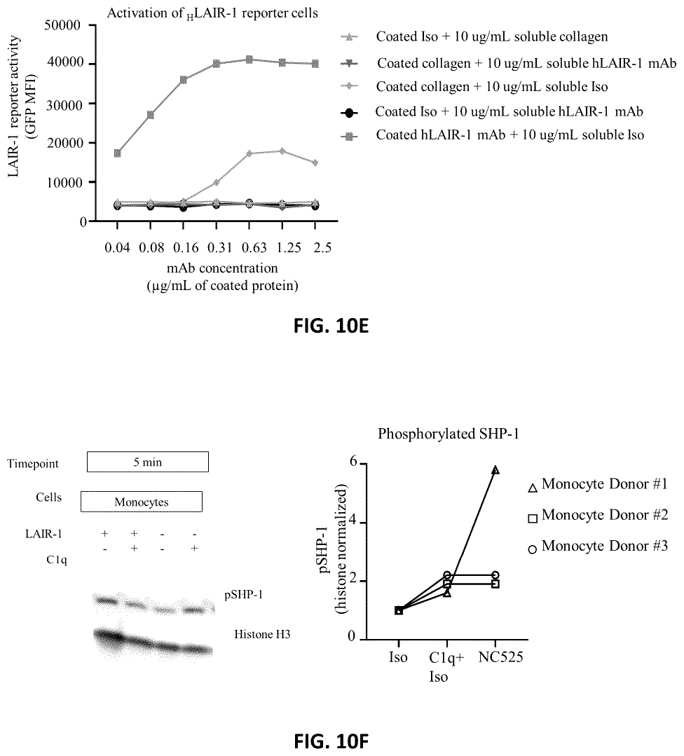

The present disclosure relates to detection of increased CAMK1/CREB activity in AML cells under LAIR-1 agonism (FIG. 10B). This may indicate that CAMK1/CREB is dispensable for AML cell survival in the context of collagen matrices, where the LAIR-1 signaling network suppresses alternative downstream mediators, such as NF-kB, MAPK, and Src kinases, to the point that the signaling threshold overcomes pro-survival mechanisms to instead result in programmed cell death.

### V. Pharmaceutical Compositions

Pharmaceutical compositions including the disclosed immunomodulatory agents are provided. Pharmaceutical compositions containing the immunomodulatory agent can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed immunomodulatory agents, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed immunomodulatory agents, generally dosage levels of 0.001 to 20 mg/kg of body weight daily are administered to mammals. Generally, for intravenous injection or infusion, dosage may be lower.

In certain embodiments, the immunomodulatory agent is administered locally, for example by injection directly into a site to be treated. Typically, the injection causes an increased localized concentration of the immunomodulatory agent composition which is greater than that which can be achieved by systemic administration. The immunomodulatory agent compositions can be combined with a matrix as described above to assist in creating an increased localized concentration of the polypeptide compositions by reducing the passive diffusion of the polypeptides out of the site to be treated.

### A. Formulations for Parenteral Administration

In some embodiments, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### B. Formulations for Oral Administration

In embodiments the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The agents can be chemically modified so that oral delivery of the derivative is efficacious. Generally, the chemical modification contemplated is the attachment of at least one moiety to the component molecule itself, where the moiety permits uptake into the blood stream from the stomach or intestine, or uptake directly into the intestinal mucosa. Also desired is the increase in overall stability of the component or components and increase in circulation time in the body. PEGylation is an exemplary chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The agent can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects.

For oral formulations, the location of release may be the stomach, the small intestine (the duodenum, the jejunem, or the ileum), or the large intestine. In some embodiments, the release will avoid the deleterious effects of the stomach environment, either by protection of the agent (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

### C. Formulations for Topical Administration

The disclosed immunomodulatory agents can be applied topically. Topical administration does not work well for most peptide formulations, although it can be effective especially if applied to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator.

Transdermal formulations may also be prepared. These will typically be ointments, lotions, sprays, or patches, all of which can be prepared using standard technology. Transdermal formulations may require the inclusion of penetration enhancers.

### D. Controlled Delivery Polymeric Matrices

The immunomodulatory agents disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of fusion polypeptides or nucleic acids encoding the fusion polypeptides, although in some embodiments biodegradable matrices are preferred. These may be natural or synthetic polymers, although synthetic polymers are preferred in some embodiments due to the better characterization of degradation and release profiles. The polymer is selected based on the period over which release is desired. In some cases, linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### VI. Therapeutic Compositions

The compositions below are to be understood as exemplary compositions related to the present disclosure. Such are not intended to be limiting of the scope of the present disclosure.

The compositions described herein can be administered to a host, either alone or in combination with a pharmaceutically acceptable excipient, in an amount sufficient to induce an appropriate anti-tumor response. The response can comprise, without limitation, specific immune response, non-specific immune response, both specific and non-specific response, innate response, primary immune response, adaptive immunity, secondary immune response, memory immune response, immune cell activation, immune cell proliferation, immune cell differentiation, and cytokine expression. LAIR-1 cell therapies can be of any of the following constructs: autologous, allogenic, universal, or armed.

An effective amount of the compositions described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the compositions. Where there is more than one administration in the present methods, the administrations can be spaced by time intervals of one minute, two minutes, three, four, five, six, seven, eight, nine, ten, or more minutes, by intervals of about 1, 2, 3, 4, 5, 6, 7, 8, 9, 10, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals, such as a priming schedule consisting of administration at 1 day, 4 days, 7 days, and 25 days, just to provide a non-limiting example. In such aspects, various compositions can be administered using different dosing and spacing regiments. In such aspects, a first composition may be administered in one or more doses spaced at certain time intervals while a second composition may be administered in a different number of doses spaced at different time intervals. In such an aspect, a first composition and second composition may differ in makeup.

The compositions of the present disclosure can be administered in a dose, or dosages, where each dose comprises at least 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 15 mg/kg, 20 mg/kg, 25 mg/kg, 30 mg/kg, 40 mg/kg, and 50 mg/kg body weight. In certain embodiments the dose preferably will comprise at least 2 mg/kg, 4.5 mg/kg, 10 mg/kg, 20 mg/kg and 30 mg/kg body weight. In yet other embodiments, the dose will most preferably comprise 10 mg/kg body weight.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

Methods for co-administration with an additional therapeutic agent are well known in the art (Hardman, et al. (eds.) (2001) Goodman and Gilman's The Pharmacological Basis of Therapeutics, 10th ed., McGraw-Hill, New York, NY; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA).

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

Formulations of therapeutic agents may be prepared for storage by mixing with physiologically acceptable carriers, excipients, or stabilizers in the form of, e.g., lyophilized powders, slurries, aqueous solutions or suspensions.

Although several aspects have been disclosed in the foregoing specification, it is understood by those skilled in the art that many modifications and other aspects will come to mind to which this disclosure pertains, having the benefit of the teaching presented in the foregoing description and associated drawings. It is thus understood that the disclosure is not limited to the specific aspects disclosed hereinabove, and that many modifications and other aspects are intended to be included within the scope of any claims that can recite the disclosed subject matter.

It should be emphasized that the above-described aspects are merely possible examples of implementations, merely set forth for a clear understanding of the principles of the present disclosure. Any process descriptions or blocks in flow diagrams should be understood as representing modules, segments, or portions of code which comprise one or more executable instructions for implementing specific logical functions or steps in the process, and alternate implementations are included in which functions may not be included or executed at all, can be executed out of order from that shown or discussed, including substantially concurrently or in reverse order, depending on the functionality involved, as would be understood by those reasonably skilled in the art of the present disclosure. Many variations and modifications can be made to the above-described aspect(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

### VII. Methods of Manufacture

### A. Methods of Making Antibodies

The antibodies can be generated in cell culture, in phage, or in various animals, including but not limited to cows, rabbits, goats, mice, rats, hamsters, guinea pigs, sheep, dogs, cats, monkeys, chimpanzees, apes. Therefore, in one embodiment, an antibody is a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); Current Protocols In Immunology (John Wiley & Sons, most recent edition).

The disclosed antibodies can be modified by recombinant means to increase greater efficacy of the antibody in mediating the desired function. Thus, it is within the scope of the invention that antibodies can be modified by substitutions using recombinant means. Typically, the substitutions will be conservative substitutions. For example, at least one amino acid in the constant region of the antibody can be replaced with a different residue. See, e.g., U.S. Pat. Nos. 5,624,821, 6,194,551, Application No. WO 9958572; and Angal, et al., Mol. Immunol. 30:105-08 (1993). The modification in amino acids includes deletions, additions, and substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to proteins, polypeptides, or fusion proteins of LAIR-1 or LAIR-2. See, e.g., Antibody Engineering: A Practical Approach (Oxford University Press, 1996).

For example, suitable antibodies with the desired biologic activities can be identified using in vitro assays including but not limited to: proliferation, migration, adhesion, soft agar growth, angiogenesis, cell-cell communication, apoptosis, transport, signal transduction, and in vivo assays such as the inhibition of tumor growth. The antibodies provided herein can also be useful in diagnostic applications. As capture or non-neutralizing antibodies, they can be screened for the ability to bind to the specific antigen without inhibiting the receptor-binding or biological activity of the antigen. As neutralizing antibodies, the antibodies can be useful in competitive binding assays.

Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic peptide. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

A monoclonal antibody is obtained from a substantially homogeneous population of antibodies, i.e., the individual antibodies within the population are identical except for possible naturally occurring mutations that may be present in a small subset of the antibody molecules. Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

Monoclonal antibodies can be made using any procedure which produces monoclonal antibodies. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

Antibodies may also be made by recombinant DNA methods. DNA encoding the disclosed antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

Methods of making antibodies using protein chemistry are also known in the art. One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, CA). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. Alternatively, the peptide or polypeptide is independently synthesized in vivo as described above. Once isolated, these independent peptides or polypeptides may be linked to form an antibody or antigen binding fragment thereof via similar peptide condensation reactions.

For example, enzymatic ligation of cloned or synthetic peptide segments allow relatively short peptide fragments to be joined to produce larger peptide fragments, polypeptides or whole protein domains. Alternatively, native chemical ligation of synthetic peptides can be utilized to synthetically construct large peptides or polypeptides from shorter peptide fragments. This method consists of a two-step chemical reaction. The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site.

### VIII. Subjects to be Treated

### A. Cancer

The disclosed compositions and methods can be used to treat cancer. Such compositions and methods can be used to treat all solid and hematologic tumors. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless explicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen (e.g., pan-carcinoma antigen (KS 1/4), ovarian carcinoma antigen (CA125), prostate specific antigen (PSA), carcinoembryonic antigen (CEA), CD19, CD20, HER2/neu, etc.).

The methods and compositions disclosed herein are useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias, chronic myelo-monocytic leukemia (CMML), high-risk myelodysplastic syndrome (MDS) and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xenoderma pegmentosum, keratoactanthoma, seminoma, thyroid follicular cancer and teratocarcinoma.

Cancers caused by aberrations in apoptosis can also be treated by the disclosed methods and compositions. Such cancers may include, but are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions in the ovary, bladder, breast, colon, lung, skin, pancreas, or uterus. In other specific embodiments, sarcoma, melanoma, or leukemia is treated or prevented by the methods and compositions.

The disclosed compositions and methods are particularly useful for the treatment of cancers that are associated with cells that express abnormally high levels of LAIR-1, high levels of LAIR-1 ligand, or a combination thereof.

Specific cancers and related disorders that can be treated or prevented by methods and compositions disclosed herein include, but are not limited to, leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK+ anaplastic large-cell lymphoma (ALCL), acute myeloid lymphoma (AML)); multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### B. Combination Therapies

The disclosed immunomodulatory agents can be administered to a subject in need thereof alone or in combination with one or more additional therapeutic agents. In some embodiments, the immunomodulatory agent and the additional therapeutic agent are administered separately, but simultaneously. The immunomodulatory agent and the additional therapeutic agent can also be administered as part of the same composition. In other embodiments, the immunomodulatory agent and the second therapeutic agent are administered separately and at different times, but as part of the same treatment regime.

The subject can be administered a first therapeutic agent 1, 2, 3, 4, 5, 6, or more hours, or 1, 2, 3, 4, 5, 6, 7, or more days before administration of a second therapeutic agent. In some embodiments, the subject can be administered one or more doses of the first agent every 1, 2, 3, 4, 5, 6 7, 14, 21, 28, 35, or 48 days prior to a first administration of second agent. The immunomodulatory agent can be the first or the second therapeutic agent.

The immunomodulatory agent and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

Exemplary molecules include, but are not limited to, cytokines, chemotherapeutic agents, radionuclides, other immunotherapeutics, enzymes, antibiotics, antivirals (especially protease inhibitors alone or in combination with nucleosides for treatment of HIV or Hepatitis B or C), anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatoires, ligands that bind to Toll-Like Receptors (including but not limited to CpG oligonucleotides) to activate the innate immune system, molecules that mobilize and optimize the adaptive immune system, other molecules that activate or up-regulate the action of cytotoxic T lymphocytes, natural killer cells and helper T-cells, and other molecules that deactivate or down-regulate suppressor or regulatory T-cells.

The additional therapeutic agents are selected based on the condition, disorder or disease to be treated. For example, the immunomodulatory agent can be co-administered with one or more additional agents that function to enhance or promote an immune response or reduce or inhibit an immune response.

### IX. Kits

The disclosed LAIR-1 immunomodulatory agents can be packaged in a hermetically sealed container, such as an ampoule or sachette, indicating the quantity. The agent can be supplied as a dry sterilized lyophilized powder or water free concentrate in a hermetically sealed container and can be reconstituted, e.g., with water or saline to the appropriate concentration for administration to a subject. For example, the agent can be supplied as a dry sterile lyophilized powder in a hermetically sealed container at a unit dosage of at least 5 mg, or at least 10 mg, at least 15 mg, at least 25 mg, at least 35 mg, at least 45 mg, at least 50 mg, or at least 75 mg. The lyophilized agent can be stored at between 2 and 8° C. in their original container and are typically administered within 12 hours, or within 6 hours, or within 5 hours, or within 3 hours, or within 1 hour after being reconstituted.

In an alternative embodiment, agent supplied in liquid form in a hermetically sealed container indicating the quantity and concentration. In some embodiments, the liquid form of the agent supplied in a hermetically sealed container including at least 1 mg/ml, or at least 2.5 mg/ml, at least 5 mg/ml, at least 8 mg/ml, at least 10 mg/ml, at least 15 mg/ml, at least 25 mg/ml, at least 50 mg/ml, at least 100 mg/ml, at least 150 mg/ml, at least 200 mg/ml of the agent.

Pharmaceutical packs and kits including one or more containers filled with agent are also provided. Additionally, one or more other prophylactic or therapeutic agents useful for the treatment of a disease can also be included in the pharmaceutical pack or kit. The pharmaceutical pack or kit can also include one or more containers filled with one or more of the ingredients of the disclosed pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

Kits designed for the above-described methods are also provided. Embodiments typically include one or more LAIR-1 immunomodulatory agents. In particular embodiments, a kit also includes one or more other prophylactic or therapeutic agents useful for the treatment of cancer, in one or more containers. In other embodiments, a kit also includes one or more anti-inflammatory agents useful for the treatment inflammatory and autoimmune diseases, in one or more containers.

### EXAMPLES

### Example 1. LAIR Antibodies and Heavy and Light Chains Sequences Thereof

### Materials and Methods

Mice were immunized with soluble human LAIR-1 (soluble LAIR-1 refers to the extracellular domain of LAIR-1) fused to a murine G2a Fc (SEQ ID NO:10). Mice were challenged with the same immunogen 2 weeks later. Mice received a 3^{rd} dose of antigen two weeks later. Three days after the final boost, mouse splenocytes were harvested and resuspended in RPMI supplemented with 10% FBS and glutamine, and later fused to form hybridomas.

RACE (Rapid Amplification of cDNA Ends) identification of the heavy and light chains was performed according to the following protocol: (1) mRNA denaturing, (2) cDNA synthesis, (3) 5′RACE Reaction, (4) analyzed PCR results (on an agarose gel to visualize the amplified DNA fragment—the correct antibody variable region DNA fragments should have a size between 500-700 base pairs, (5) TOPO cloned PCR positive bands; (6) PCR-amplified TOPO clones, followed by gel electrophoresis and recovery from agarose gel, (7) sequenced 218 clones in total, (8) performed CDR analysis using sequencing data (CDR regions were defined using VBASE2 available through vbase2.org).

### Results

In the following amino acid sequences, the underlined portions of the sequences are the complementarity-determining regions (CDRs).

10D6 SEQUENCES
10D6 VL
(SEQ ID NO: 7)
DIQMTQSPASQSASLGESVTITCLASQTIGTWLAWFQQKPGKSPQLLIYAA
TSLADGVPSRFSGSGSGTKFSFKISSLQAEDFVSYYCQQLYSAPYTFGGGTKLEIK*
c10D6 Light Chain (human kappa constant domain)
(SEQ ID NO: 8)
DIQMTQSPASQSASLGESVTITCLASQTIGTWLAWFQQKPGKSPQLLIYAA
TSLADGVPSRFSGSGSGTKFSFKISSLQAEDFVSYYCQQLYSAPYTFGGGTKLEIK
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC*
10D6 VH
(SEQ ID NO: 9)
EVHLVETGGGLVQPKGSLKLSCAASGFNFNTNAMNWVRQAPGKGLEWV
ARIRTKSNNYATYYADSVKDRFTISRDDSQSMLYLQMNNLKTEDTAMYYCVSTP
YFTYWGQGTLVTVSA*
c10D6 Heavy Chain (human IgG1 constant domain)
(SEQ ID NO: 10)
EVHLVETGGGLVQPKGSLKLSCAASGFNFNTNAMNWVRQAPGKGLEWV
ARIRTKSNNYATYYADSVKDRFTISRDDSQSMLYLQMNNLKTEDTAMYYCVSTP
YFTYWGQGTLVTVSAASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHE
ALHNHYTQKSLSLSPG*
c10D6 Heavy Chain (human IgG4 constant domain)
(SEQ ID NO: 11)
EVHLVETGGGLVQPKGSLKLSCAASGFNFNTNAMNWVRQAPGKGLEWV
ARIRTKSNNYATYYADSVKDRFTISRDDSQSMLYLQMNNLKTEDTAMYYCVSTP
YFTYWGQGTLVTVSAASTKGPSVFPLAPCSRSTSESTAALGCLVKDYFPEPVTVS
WNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDP
EVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALH
NHYTQKSLSLSLG*
11B3 SEQUENCES
11B3 VL
(SEQ ID NO: 12)
DIQMAQSSSSFSVSLGDRVTITCKASEDIYIRLAWYQQKPGNAPRLLISTAT
SLETGVPSRFSGSGSGKDYTLSITSLQTEDVATYYCQQYWSTPYTFGGGTRLEIK
c11B3 Light Chain (human kappa constant domain)
(SEQ ID NO: 13)
DIQMAQSSSSFSVSLGDRVTITCKASEDIYIRLAWYQQKPGNAPRLLISTAT
SLETGVPSRFSGSGSGKDYTLSITSLQTEDVATYYCQQYWSTPYTFGGGTRLEIKR
TVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC*
h11B3 VL-1
(SEQ ID NO: 14)
DIQMTQSPSSLSASVGDRVTITCKASEDIYIRLAWYQQKPGKAPRLLISTAT
SLETGVPSRFSGSGSGTDYTFTISSLQPEDIATYYCQQYWSTPYTFGGGTRLEIK*
h11B3 VL-1 Light Chain (kappa constant domain)
(SEQ ID NO: 15)
DIQMTQSPSSLSASVGDRVTITCKASEDIYIRLAWYQQKPGKAPRLLISTAT
SLETGVPSRFSGSGSGTDYTFTISSLQPEDIATYYCQQYWSTPYTFGGGTRLEIKRT
VAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC*
h11B3 VL-2
(SEQ ID NO: 16)
DIQMTQSPSSLSASLGDRVTITCKASEDIYIRLAWYQQKPGKAPRLLISTAT
SLETGVPSRFSGSGSGTDYTFTISSLQPEDIATYYCQQYWSTPYTFGGGTRLEIK*
h11B3 VL-2 Light Chain (kappa constant domain)
(SEQ ID NO: 17)
DIQMTQSPSSLSASLGDRVTITCKASEDIYIRLAWYQQKPGKAPRLLISTAT
SLETGVPSRFSGSGSGTDYTFTISSLQPEDIATYYCQQYWSTPYTFGGGTRLEIKRT
VAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC*
11B3 VH
(SEQ ID NO: 18)
EVQLVESGGGLVQPKGSLKLSCAASGFTFNTNAMYWVRQAPGKGLEWV
ARIRSKSSNYATYYADSVKDRFTISRDDSQSMLYLQMNNLKTEDTARYYCVRGG
SGFFAYWGQGTLVTVSA*
c11B3 Heavy Chain (human IgG1 constant domain)
(SEQ ID NO: 19)
EVQLVESGGGLVQPKGSLKLSCAASGFTFNTNAMYWVRQAPGKGLEWV
ARIRSKSSNYATYYADSVKDRFTISRDDSQSMLYLQMNNLKTEDTARYYCVRGG
SGFFAYWGQGTLVTVSAASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
c11B3 Heavy Chain (human IgG4 constant domain)
(SEQ ID NO: 20)
EVQLVESGGGLVQPKGSLKLSCAASGFTFNTNAMYWVRQAPGKGLEWV
ARIRSKSSNYATYYADSVKDRFTISRDDSQSMLYLQMNNLKTEDTARYYCVRGG
SGFFAYWGQGTLVTVSAASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-1
(SEQ ID NO: 21)
EVQLVESGGGLVQPGGSLKLSCAASGFTFNTNAMYWVRQAPGKGLEWV
ARIRSKSSNYATYYAASVKGRFTISRDDSKNTLYLQMNSLKTEDTAVYYCVRGG
SGFFAYWGQGTLVTVSS*
h11B3 VH-1 Heavy Chain (IgG1 constant domain)
(SEQ ID NO: 22)
EVQLVESGGGLVQPGGSLKLSCAASGFTFNTNAMYWVRQAPGKGLEWV
ARIRSKSSNYATYYAASVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-1 Heavy Chain (IgG4 constant domain)
(SEQ ID NO: 23)
EVQLVESGGGLVQPGGSLKLSCAASGFTFNTNAMYWVRQAPGKGLEWV
ARIRSKSSNYATYYAASVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-2
(SEQ ID NO: 24)
EVQLVESGGGLVQPGGSLKLSCAASGFTFSTQAMYWVRQAPGKGLEWV
ARIRSKSSNYATYYAASVKGRFTISRDDSKNTLYLQMNSLKTEDTAVYYCVRGG
SGFFAYWGQGTLVTVSS*
h11B3 VH-2 Heavy Chain (IgG1 constant domain)
(SEQ ID NO: 25)
EVQLVESGGGLVQPGGSLKLSCAASGFTFSTQAMYWVRQAPGKGLEWV
ARIRSKSSNYATYYAASVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-2 Heavy Chain (IgG4 constant domain)
(SEQ ID NO: 26)
EVQLVESGGGLVQPGGSLKLSCAASGFTFSTQAMYWVRQAPGKGLEWV
ARIRSKSSNYATYYAASVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-3
(SEQ ID NO: 27)
EVQLVESGGGLVQPGGSLKLSCAASGFTFNTNAMYWVRQAPGKGLEWV
GRIRSKSSNYATYYAASVKGRFTISRDDSKNTLYLQMNSLKTEDTAVYYCVRGG
SGFFAYWGQGTLVTVSS*
h11B3 VH-3 Heavy Chain (IgG1 constant domain)
(SEQ ID NO: 28)
EVQLVESGGGLVQPGGSLKLSCAASGFTFNTNAMYWVRQAPGKGLEWV
GRIRSKSSNYATYYAASVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-3 Heavy Chain (IgG4 constant domain)
(SEQ ID NO: 29)
EVQLVESGGGLVQPGGSLKLSCAASGFTFNTNAMYWVRQAPGKGLEWV
GRIRSKSSNYATYYAASVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-4
(SEQ ID NO: 30)
EVQLVESGGGLVQPGGSLKLSCAASGFTFNTNAMYWVRQAPGKGLEWV
ARIRSKSSNYATYYADSVKGRFTISRDDSKNTLYLQMNSLKTEDTAVYYCVRGG
SGFFAYWGQGTLVTVSS*
h11B3 VH-4 Heavy Chain (IgG1 constant domain)
(SEQ ID NO: 31)
EVQLVESGGGLVQPGGSLKLSCAASGFTFNTNAMYWVRQAPGKGLEWV
ARIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-4 Heavy Chain (IgG4 constant domain)
(SEQ ID NO: 32)
EVQLVESGGGLVQPGGSLKLSCAASGFTFNTNAMYWVRQAPGKGLEWV
ARIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-5
(SEQ ID NO: 33)
EVQLVESGGGLVQPGGSLKLSCAASGFTFNTNAMYWVRQAPGKGLEWV
GRIRSKSSNYATYYADSVKGRFTISRDDSKNTLYLQMNSLKTEDTAVYYCVRGG
SGFFAYWGQGTLVTVSS*
h11B3 VH-5 Heavy Chain (IgG1 constant domain)
(SEQ ID NO: 34)
EVQLVESGGGLVQPGGSLKLSCAASGFTFNTNAMYWVRQAPGKGLEWV
GRIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-5 Heavy Chain (IgG4 constant domain)
(SEQ ID NO: 35)
EVQLVESGGGLVQPGGSLKLSCAASGFTFNTNAMYWVRQAPGKGLEWV
GRIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-6
(SEQ ID NO: 36)
EVQLVESGGGLVQPGGSLKLSCAASGFTFSTNAMYWVRQAPGKGLEWV
GRIRSKSSNYATYYADSVKGRFTISRDDSKNTLYLQMNSLKTEDTAVYYCVRGG
SGFFAYWGQGTLVTVSS*
h11B3 VH-6 Heavy Chain (IgG1 constant domain)
(SEQ ID NO: 37)
EVQLVESGGGLVQPGGSLKLSCAASGFTFSTNAMYWVRQAPGKGLEWV
GRIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-6 Heavy Chain (IgG4 constant domain)
(SEQ ID NO: 38)
EVQLVESGGGLVQPGGSLKLSCAASGFTFSTNAMYWVRQAPGKGLEWV
GRIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-7
(SEQ ID NO: 39)
EVQLVESGGGLVQPGGSLKLSCAASGFTFNTQAMYWVRQAPGKGLEWV
GRIRSKSSNYATYYADSVKGRFTISRDDSKNTLYLQMNSLKTEDTAVYYCVRGG
SGFFAYWGQGTLVTVSS*
h11B3 VH-7 Heavy Chain (IgG1 constant domain)
(SEQ ID NO: 40)
EVQLVESGGGLVQPGGSLKLSCAASGFTFNTQAMYWVRQAPGKGLEWV
GRIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-7 Heavy Chain (IgG4 constant domain)
(SEQ ID NO: 41)
EVQLVESGGGLVQPGGSLKLSCAASGFTFNTQAMYWVRQAPGKGLEWV
GRIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-8
(SEQ ID NO: 42)
EVQLVESGGGLVQPGGSLKLSCAASGFTFSTQAMYWVRQAPGKGLEWV
GRIRSKSSNYATYYADSVKGRFTISRDDSKNTLYLQMNSLKTEDTAVYYCVRGG
SGFFAYWGQGTLVTVSS*
h11B3 VH-8 Heavy Chain (IgG1 constant domain)
(SEQ ID NO: 43)
EVQLVESGGGLVQPGGSLKLSCAASGFTFSTQAMYWVRQAPGKGLEWV
GRIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*
h11B3 VH-8 Heavy Chain (IgG4 constant domain)
(SEQ ID NO: 44)
EVQLVESGGGLVQPGGSLKLSCAASGFTFSTQAMYWVRQAPGKGLEWV
GRIRSKSSNYATYYADSVKGRFTISRDDSKNTLYLQMNSLKTEDTAVYYCVRGG
SGFFAYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEA
LHNHYTQKSLSLSLG*

A number of humanized anti-LAIR-1 immunomodulatory variants have been synthesized using combinations of the above provided sequences. Non-limiting examples of such synthesized variants, and sequence identifiers associated with such exemplary variants, are provided in Table 1 below.

TABLE 1
Anti-LAIR-1 Variants

| Anti-LAIR-1 | | |
|---|---|---|
| Variant | Variable Light Chain | Variable Heavy Chain |
| Anti-LAIR-1 | 11B3 VL (SEQ ID NO: 12) | 11B3 VH (SEQ ID NO: 18) |
| 1 | h11B3 VL-1 (SEQ ID NO: 14) | h11B3 VH-1 (SEQ ID NO: 21) |
| 2 | h11B3 VL-2 (SEQ ID NO: 16) | h11B3 VH-1 (SEQ ID NO: 21) |
| 3 | h11B3 VL-1 (SEQ ID NO: 14) | h11B3 VH-2 (SEQ ID NO: 24) |
| 4 | h11B3 VL-2 (SEQ ID NO: 16) | h11B3 VH-2 (SEQ ID NO: 24) |
| 5 | h11B3 VL-1 (SEQ ID NO: 14) | h11B3 VH-3 (SEQ ID NO: 27) |
| 6 | h11B3 VL-2 (SEQ ID NO: 16) | h11B3 VH-3 (SEQ ID NO: 27) |
| 7 | h11B3 VL-1 (SEQ ID NO: 14) | h11B3 VH-4 (SEQ ID NO: 30) |
| 8 | h11B3 VL-2 (SEQ ID NO: 16) | h11B3 VH-4 (SEQ ID NO: 30) |
| 9 | h11B3 VL-1 (SEQ ID NO: 14) | h11B3 VH-5 (SEQ ID NO: 33) |
| 10 | h11B3 VL-2 (SEQ ID NO: 16) | h11B3 VH-5 (SEQ ID NO: 33) |
| 11 | h11B3 VL-1 (SEQ ID NO: 14) | h11B3 VH-6 (SEQ ID NO: 36) |
| 12 | h11B3 VL-2 (SEQ ID NO: 16) | h11B3 VH-6 (SEQ ID NO: 36) |
| 13 | h11B3 VL-1 (SEQ ID NO: 14) | h11B3 VH-7 (SEQ ID NO: 39) |
| 14 | h11B3 VL-2 (SEQ ID NO: 16) | h11B3 VH-7 (SEQ ID NO: 39) |
| 15 | h11B3 VL-1 (SEQ ID NO: 14) | h11B3 VH-8 (SEQ ID NO: 42) |
| 16 | h11B3 VL-2 (SEQ ID NO: 16) | h11B3 VH-8 (SEQ ID NO: 42) |

### Example 2

### Methods and Materials

### Patient Samples

For ex vivo studies, AML patient or healthy donor whole blood was purchased through StemExpress (Folsom, CA). Whole blood was collected into EDTA Vacutainer tubes, kept chilled, and tested within 24 hours of draw.

### Animals

NSG (NOD-scid-IL2Rgammanull) and NSG-SGM3 (NOD.Cg-PrkdcscidIl2rgtm1WjlTg(CMV-IL3,CSF2,KITLG)1Eav/MloySzJ) mice were purchased from Jackson Laboratory.

### Flow Cytometry

All human cell preparations were more than 95% viable by trypan blue exclusion. 1e6 thawed or fresh bone-marrow mononuclear cells were stained with Zombie Aqua viability dye, Annexin-5-AlexaFlour647, and mAbs conjugated to FITC, PE-Cy7, BV785, BV421, APC-Cy7, BV605, PerCP-Cy5.5, or Alexa Flour647 specific for human lineage markers CD34, (BD Biosciences, San Diego, CA), CD38, CD90, CD45RA, CD123, CD117 (BioLegend, San Diego, CA), or human LAIR-1 (Clone DX26, BD Biosciences, San Diego, CA), respectively. After staining, cells were washed, re-suspended in phosphate-buffered saline (PBS) with 1-4% paraformaldehyde, and analyzed in a Celesta flow cytometer (BD Biosciences, San Diego, CA) or an Attune NXT flow cytometer (Thermofisher, Waltham, MA) and analyzed using FlowJo software (Treestar, San Carlos, CA).

For most of the analyses, at least 3e5 total events were analyzed, with sequential gating of bone-marrow mononuclear cells. For CD34⁺ AML cells, lineage negative cells were demarcated as CD34⁺CD38⁻CD45RA⁻CD90⁻(MPP-like leukemia stem cells (LSCs)), CD34⁺CD38⁻CD45RA⁺CD90⁻ (LMPP-like LSCs), CD34⁺CD38⁺CD45RA⁺CD123⁺ (GMP-like LSCs). For CD34⁻ AML cells, lineage negative cells were demarcated as CD34⁻ CD117⁺ (GM precursor-like LSCs) (Thomas and Majeti et al. Blood 2017; 129:1577). LAIR-1 expression was assessed in each LSC subset compared with isotype control. For healthy donor bone marrow, the same mAb-conjugates were used except PerCP-Cy5.5 CD127 (Biolegend, San Diego, CA) was substituted for PerCP-Cy5.5 CD117 in the AML panel. Healthy donor bone marrow cells were gated according to the recommendations from Pang et al. [REF: PNAS 2011; 108:20012; PMID: 22123971]. HSPCs were demarcated as CD34⁺CD38⁻CD45RA⁻CD90⁺ (HSCs), CD34⁺CD38⁻CD45RA⁻CD90⁻ (MPPs), CD34⁺CD38⁺CD127⁺ (CLPs), CD34⁺CD38⁺CD45RA⁻CD123⁺(CMPs), CD34⁺CD38⁺CD45RA⁻CD123⁻ (MEPs), and CD34⁺CD38⁺CD45RA⁺CD123⁺ (GMPs). LAIR-1 expression was assessed on each HSPC subset compared with isotype control. In both AML and healthy donor bone marrow cell analyses, fluorescence minus one was used to separate CD45RA⁺ from CD45RA⁻, CD90⁺ from CD90⁻, CD123⁺ from CD123⁻, and CD127⁺ from CD127− population.

MV4-11-LAIR-1^{KO} and MV-4-11^{WT} cells were used to validate the specificity of LAIR-1 mAbs. AML cell lines MV4-11, THP-1, HL-60 and U937 were purchased from ATCC; Kasumi1, NB4, HEL1 (gifts from Manoj Pillai, Yale University), and MOLM14 (gift from Martin Carroll, University of Pennsylvania) were used as described.

### TCGA Analysis

Normalized RNA-seq by Expectation and Maximization (RSEM) mRNA expression data on 162 samples from AML patients included in the Cancer Genome Atlas (TCGA) project were downloaded from www.cbioportal.org (hosted by Memorial Sloan Kettering Cancer Center) together with information on corresponding clinical, mutational, and cytogenetic parameters.

### Antibody Cell Binding and Reporter Cell Assays

For cell binding assays, 5e4 UT-140 cells or mouse LAIR-1-transfected 293T cells per well were plated in 200 μL PBS in a 96-well round bottom plate, centrifuged for 4 minutes at 500×g for a single wash, and blocked for 10 minutes on ice with flow cytometry buffer (PBS+2% FBS+0.1 mM EDTA) containing 1:50 dilution of TruStain™ Fc block (Biolegend, San Diego, CA). Cells were subsequently stained for 30 min on ice with titrated concentrations of soluble AF647-labeled LAIR-1 mAb or AF647-labeled isotype control. Cells were then washed 3 times as previously described and binding was measured by flow cytometry using an Attune NXT flow cytometer. For reporter cell assays, flat-bottom 96-well TC plates were coated with 10 μg/mL per well of the indicated antibody in PBS or 2 μg/mL of ligand in 0.01N HCl overnight at 4° C. Wells were washed once with 200 μL sterile PBS. 5e4-to-1e5 cells per well were plated in 100 μL RPMIc containing titrated concentrations of LAIR-1 mAb or isotype control, then incubated overnight at 37° C. The next day, cells were transferred to a 96-well round-bottom plate, centrifuged for 4 minutes at 500×g, and resuspended in 200 μL of flow cytometry buffer. Cells were then centrifuged and resuspended for a total of 3 washes. GFP expression was measured by flow cytometry using an Attune NXT Flow Cytometer.

### Colony Forming Unit Assay

Cryopreserved AML bone-marrow cells or healthy donor CD34 cells were thawed and plated in 96 wells plates. Cells were engaged with the indicated concentrations of LAIR-1 mAb or isotype control for 30 min at room temperature. Subsequently, cells were diluted in IMDM with 2% FBS and mixed with semisolid methylcellulose-based medium (MethoCult H4435 Enriched, StemCell Technologies, Vancouver, Canada) which contains human cytokines (stem cell factor, IL-3, IL-6, EPO, G-CSF, GM-CSF). Then, between 1e4 and 2.5e5 LSCs, or, between 1e3 and 5e3 healthy donor CD34 cells per well were plated on 6-well SmartDishes (StemCell Technologies, Vancouver, Canada). The cell-loaded plates were incubated at 37° C. with 5% CO2 in air and ≥95% humidity. On day 14, colony formation was counted by STEMvision (StemCell Technologies, Vancouver, Canada), an automated and standardized colony counting instrument. The automated results were confirmed and edited manually by the STEMvision Colony Marker software.

### Patient-Derived Xenograft (PDX) Models

Newborn (3-5 day from birth) NSG-SGM3 pups were sub-lethally (200 cGy) irradiated. 12 hours post-irradiation, mice were intra-hepatically transplanted with 0.2e6 AML bone-marrow cells or normal CD34 HSCs from cryopreserved stocks. 6 weeks post-transplant, blood was collected to assess engraftment of progenitor cells via flow cytometry, staining for human CD45, human CD33, and human CD3. Once engraftment of human cells was confirmed, 5 mg/kg of anti-human LAIR-1 mAb or isotype control was intraperitoneally injected weekly for a total of 4 doses. At the indicated timepoints post-treatment, blood was collected to assess the proliferation of AML cells as a measure of LAIR-1 mAb anti-leukemic activity as compared to isotype control. For standard-of-care combination studies, PDX mice were treated with 20 mg/kg venetoclax 5 days on and 2 days off for 5 weeks, and X 1.5 mg/kf-tiw azacytidine upfront one week only. LAIR-1 mAb was treated i.p. once per week for 5 weeks. Blood was harvested as described above to measure leukemic growth and spleens were harvested at endpoint and weighed for assessment of splenomegaly. For secondary transplant studies, mice were engrafted as above. Once engraftment of human cells was confirmed, 5 mg/kg anti-human LAIR-1 mAb or isotype control was intraperitoneally injected weekly for a total of 4 doses. Bone-marrow from treated mice was transplanted into naive, sub-lethally irradiated NSG-SGM3 recipient mice. No treatment was provided to recipient mice. Leukemia progression in recipient mice was measured by flow cytometry by quantifying the percent of human CD33⁺ human CD45⁺ cells in peripheral blood at weeks 4, 6, and 10 weeks post-transplant.

For assessment of mAb impacts on normal human immune cells, NSG-SGM3-CD34⁺ fully engrafted humanized mice (>25% human CD45+ leukocytes in circulation) were purchased from Jackson Laboratory (stock #2523, Bar Harbor, ME). Mice were received approximately 12 weeks after engraftment and experiment was initiated after 1 week acclimatization. Mice were treated with 5 mg/kg mAb by i.p. weekly for 4 weeks. One week after the final mAb dose, mice were euthanized and splenocytes, lymph nodes, and bone marrow cells were harvested for total cell counts, followed by analysis of CD45⁺, CD3⁺, CD4⁺, CD8⁺, CD14⁺, CD11b⁺ CD20⁺, CD56⁺ cells percent. Absolute numbers of cell subpopulations were calculated, as well as percentages of each population subset as a percentage of total CD45⁺ cells and total leukocyte gate.

### Cell-Derived Xenograft (CDX) Models

NSG mice were injected with 2e6 THP-1 luciferase cells or 2e6 MV4-11-luciferase cells via tail vein injection. Leukemia progression was quantified by i.p. injection of 100 μL bioluminescent substrate and IVIS imaging 8 minutes post injection. Progression was assessed weekly staring on Day 7 post-challenge. 10 mg/kg mab treatment was performed by i.p injection starting on day 8 and continuing twice a week until the end of the study. For the subcutaneous engraftment model, NSG mice were subcutaneously injected on the right flank with 5e6 MV4-11-Luc-LAIR-1^{OE} cells. Mice were treated by weekly i.p. injection of 10 mg/kg mAb. Tumor growth was measured by calipers twice weekly until end point.

### Ex Vivo Depleting Assay

Fresh whole blood was RBC-depleted using the EasySep™ RBC Depletion Reagent (StemCell, Vancouver, BC) following the manufacturer's protocol. Alternatively, frozen PBMCs were thawed in pre-warmed cRPMI media. Cell number was determined by VI-Cell XR cell counter. 2e5 cells per well were plated into 96-well plates lacking or containing 50 μg/mL pre-coated human Collagen I (StemCell, Vancouver, BC) in 200 μL of cRPMI containing 10 μg/mL of soluble LAIR-1 mAb or isotype control antibody (NP782 (NextCure, Beltsville, MD) or InVivoMAb recombinant human IgG1 Fc (BioXcell, Lebanon, NH)). Plates were centrifuged at 100×g for 2 minutes, then incubated for 20 hours at 37° C. in a TC incubator. At the end of incubation period, plates were centrifuged at 500×g for 4 min. Cells were resuspended and then transferred to a 96-well round-bottom plate for cell staining and flow cytometry as described above.

### Western Blots and Phospho-Arrays

For assessment of signaling in healthy cells, human monocytes were isolated from PBMCs from two donors using a StemCell monocyte isolation kit (StemCell, Vancouver, BC). Cells seeded in cRPMI at 2e6 cells per well of a 12-well plate were stimulated with 20 ng/mL C1q (CompTech) with or without 10 μg/mL of LAIR-1 mAb or isotype control antibody (NextCure). The plate was centrifuged at 350×g for 2 min. After a 5 minute incubation in a 37° C. TC incubator, cells were pelleted, lysed in the presence of phosphatase inhibitors, and processed for Western blotting. Equal amounts of protein from each sample were separated on a gradient gel, transferred to PVDF, blocked in 5% BSA, and probed with pSHP-1 or Histone H3 antibodies (D11G5 and D1H2, respectively, Cell Signaling Technology, MA). Data were quantified using FIJI (NIH, MD).

### Digital Spatial Imaging and Quantification

Protocols and reagents from NanoString were used for GeoMx digital spatial imaging (DSP). Briefly, formalin-fixed paraffin-embedded (FFPE) sections of tumor and spleen were deparaffinized and rehydrated, antigen-retrieved in citrate buffer, blocked, and stained with the Human Immune Profiling Core and Cell Death Panel overnight at 37 degrees C. Anti-HCD45-Alexaflour 647 antibody (NBP2-34528AF647 from Novus Biologicals, Centennial, CO) was used as a morphology marker at 5 μg/mL to identify MV4-11 cells. After washing, slides were fixed in 4% paraformaldehyde and nuclei were stained with Syto13. Slides were scanned and regions of interest (human CD45 regions) were acquired on a GeoMx DSP instrument (Nanostring, Seattle, WA). Approximately 5 regions of interest (ROIs) were collected per tissue, and samples were multi-plexed and processed on the nCounter Prep Station and Digital Analyzer as recommended. Data were analyzed on DSP Analysis software. Following quality control, data were normalized to housekeeping genes, and statistics were performed using a linear mixed model with a Benjamin-Hochberg correction.

### Results

### LAIR-1 Agonism Inhibits Growth of Bone Marrow LSC's

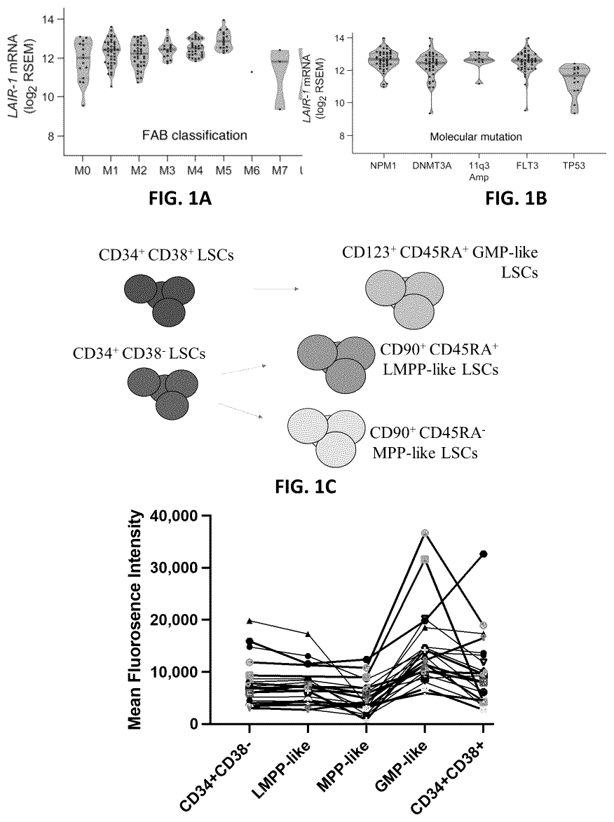

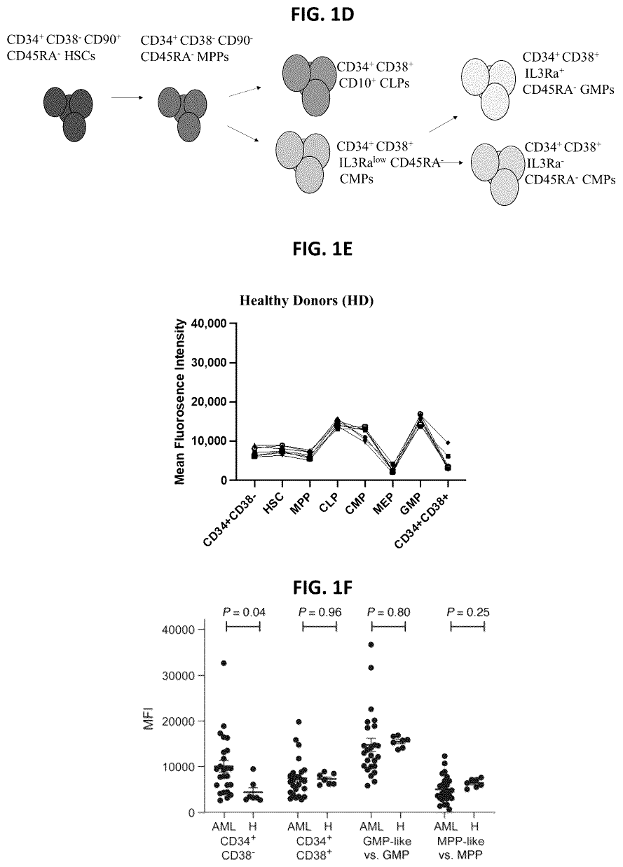

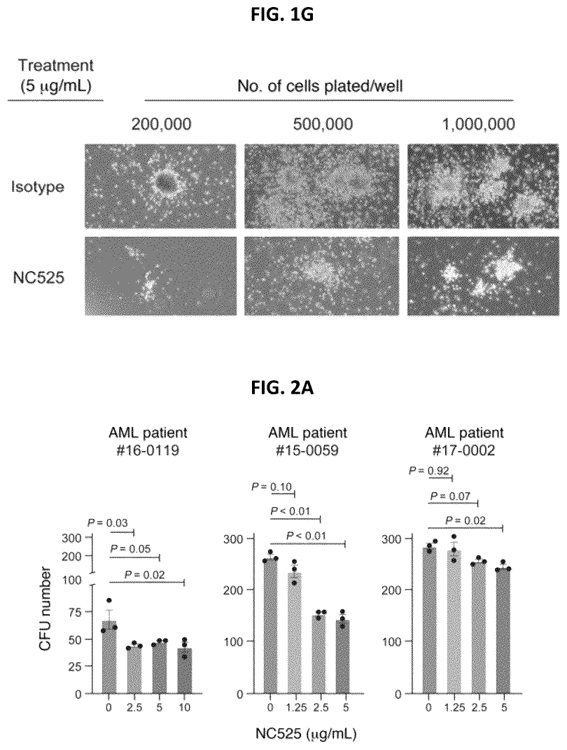

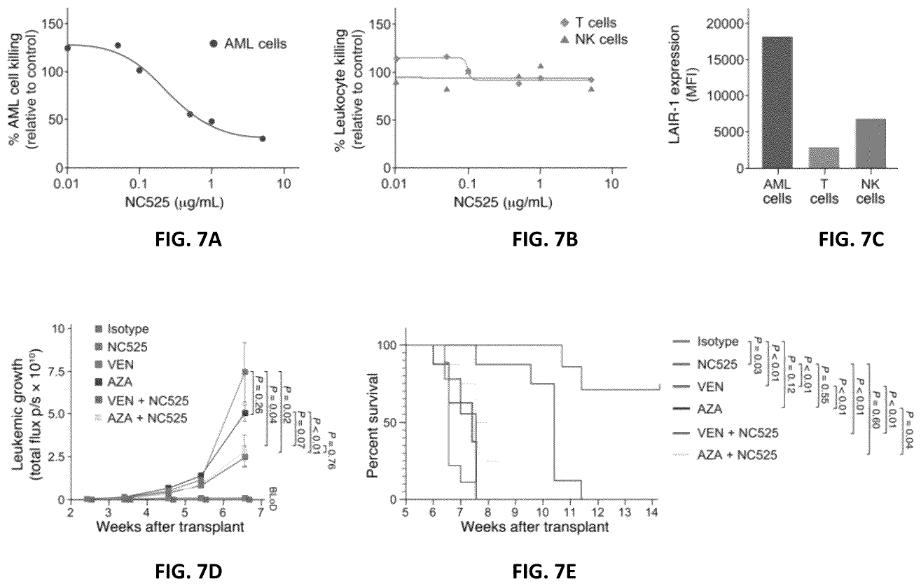

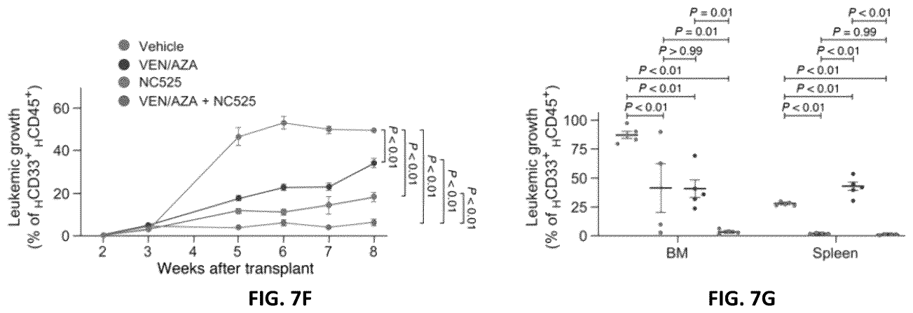

Studies have reported aberrantly elevated LAIR-1 expression on leukemic cells (Kang, Lu et al., 2015, Ramos et al., 2021). To extend this finding, the expression of LAIR-1 on human AML was assessed. The Cancer Genome Atlas was analyzed for LAIR1 mRNA levels from AML patients representing each disease subtype as described by the French-American-British (FAB) classification system (M0-M7), and likewise for expression of LAIR1 in patients with AML associated mutations. No difference was observed in LAIR1 between various subtypes, with each subtype except M7 having higher mean expression than undiagnosed donors (FIG. 1A). Similarly, LAIR1 mRNA levels did not correlate with any particular mutations (FIG. 1B). Because leukemic blasts in the peripheral blood arise from a pool of self-renewing LSCs within the bone-marrow, it was determined cell surface expression of LAIR-1 on different lineage subsets of human AML cells (Thomas and Majeti, 2017; Seita and Weissman, 2010) (FIG. 1C) using flow cytometry (FIG. 7C). LAIR-1 levels were variable across AML patients, with GMP-like and CD34 CD38 subsets expressing the highest overall levels of LAIR-1 receptor (FIG. 1D). Conversely, healthy donor HSC subsets (FIG. 1E) displayed very little variability (FIG. 1F). Consistent with the mRNA data, the CD34 CD38 subset from AML donors displayed higher LAIR-1 expression than healthy donors (FIG. 1G).

LAIR-1 mAb is a humanized mAb with a functional IgG1 backbone that specifically binds to human LAIR-1, but not mouse LAIR-1 (FIG. 10A-10C), and blocks collagen binding to LAIR-1 (FIG. 10D). LAIR-1 mAb is capable of inducing human LAIR-1 signaling (agonist) upon engagement and crosslinking (FIG. 10E-10F).

### LAIR-1 Agonism Inhibits Growth of Bone Marrow LSC's but not Healthy HSC's

To test the effect of LAIR-1 engagement on LSCs, ex vivo colony forming unit (CFU) assays were performed with AML bone-marrow recovered from multiple patients, as shown in Table 2 below.

TABLE 2
AML Patient Samples

| | Patient | Sample | Types | BM | | |
|---|---|---|---|---|---|---|
| | ID | ID | of AML | blast % | Karyotype | NGS data |
| AML01 | 14-0026 | 14-04-008 | NK | 89.5 | 46, XX[20] | Not done |
| AML02 | 19-0089 | 19-07-014F | FLT3 | 97.5 | 46, XY, t(1; 4)(p36.1; q31)[7]/46, | FLT3 ITD p.Val581_Phe594dup |
| | | | mutated | | idem, del(7)(q34)[3]/48, | (47%) |
| | | | | | XY, +8, +13[10]7 | |
| | | | | | | IDH2 p.Arg172Lys (96%) |
| AML03 | 17-0002 | 17-01-005 | CK | 97 | 47, XX, −8, −13, | FLT3 ITD |
| | | | without | | ?del(18)(q21), +21, +2mar[5]/46, | p.Arg595_Leu601dup, and splice |
| | | | monosomy | | XX, add(21)(q22)[2]/46, XX[14] | site variant, p.?. WT1: |
| | | | | | | p.Ala170tyrfs*70 and p.Ser169* |
| AML04 | 20-0164 | 20-12-004F | t(8; 21) | 62 | 46, XX, t(8; 21)(q22; q22)[20] | Tier II: Variants of Potential |
| | | | | | | Clinical Significance |
| | | | | | | EZH2 |
| | | | | | | p.Arg63Profs*10 (10%) |
| | | | | | | Tier III: Variants of Unknown |
| | | | | | | Clinical Significance |
| | | | | | | EZH2 |
| | | | | | | p.Gly743_Ile744insGlyGly (7%) |
| | | | | | | RUNX1 p.Ser172Asn (11%) |
| AML05 | 14-0016 | 14-03-015 | inv(16) | 63 | 46, XY, der(7)t(7; 16)(q22; | NGS not done. |
| | | | | | q22)inv(16)(p13.1q22), der(16)t(7; | |
| | | | | | 16)inv(16)(p13.1q22)[18]/46, | |
| | | | | | XY[2] | |
| | | | | | | AML Snapshot Panel: No |
| | | | | | | mutations detected in IDH1, |
| | | | | | | IDH2, FLT-3 (TKD), KIT |
| | | | | | | No mutations detected in FLT3, |
| | | | | | | NPM1, BEBPA |
| AML06 | 21-0043 | 21-04-006F | TP53 | 31.5 | 44, XX, del(5)(q22q35), | TP53 p.Cys242Ser (90%) |
| | | | mutated | | add(12)(p13), −13, | |
| | | | | | add(13)(p11.2), −16[cp9]/48, | |
| | | | | | idem, +X, +8, add(13)(p11.2), +22[10]/46, | |
| | | | | | XX[1] | |
| AML07 | 20-0052 | 20-03-018F | FLT3 | 77 | 46, XY, +1, der(1; 15)(q10; | CSF3R p.Thr618Ile (38%) |
| | | | mutated | | q10)[9]/43~46, XY, | |
| | | | | | i(1)(q10)[cp2]/46, XY[9] | |
| | | | | | | FLT3-TKD p.Asp835Tyr (43%) |
| | | | | | | SRSF2 p.Pro95Arg (50%) |
| | | | | | | TET2 |
| | | | | | | p.Leu1332_Met1333delinsProLeu(45%) |
| | | | | | | ZRSR2 p.Arg126* (81%) |
| AML08 | AAA154 | 110520B | Monocytic | 87.5 | 46, XX[20] | NGS not done. |
| | | | leukemia | | | No mutations found in FLT3, |
| | | | | | | CEBPA, cKIT |
| AML09 | 16-0119 | 16-05-012 | t(8; 21) | 35 | 45, X, −Y, t(8; 21)(q22; q22)[5]/45, | One insertion mutation in |
| | | | | | idem, del(9)(q21q22)[9]/46, | KIT(p.Tyr418_Asp419insPhePhe) |
| | | | | | XY[6] | was identified in this patient's |
| | | | | | | sample |
| AML10 | AAA295 | 120927B | inv(16) | 83.5 | 46, XX, del(7)(q22), del(9)(p22), | NGS not done. |
| | | | | | del(12)(p12), | |
| | | | | | inv(16)(p13.1q22)[cp20] | |
| | | | | | | No mutations found in FLT3, |
| | | | | | | NPM1, CEBPA. cKIT positive. |
| AML11 | 15-0059 | 15-05-008 | NK | 94 | 46, XY[20] | NGS not done. No mutations in |
| | | | | | | IDH1/2, FLT3-TKD, DNMT3A |
| | | | | | | or KIT. FLT3-ITD positive |
| | | | | | | mutant: wild type ratio is 0.15. |
| AML12 | AAA318 | 130321A | inv(16) | 90 | 46, XX, inv(16)(p13.1q22)[17]/46, | NGS not done. |
| | | | | | XX[3] | |
| | | | | | | Negative for mutations in FLT3, |
| | | | | | | NPM1, CEBPA, cKIT |
| AML13 | 15-0002 | 15-01-002 | t(8; 21) | 70 | 45, X, −X, t(8; 21)(q22; | NGS not done. |
| | | | | | q22)[19]/46, | |
| | | | | | XX[1] | |
| | | | | | | Negative for mutations in FLT3, |
| | | | | | | NPM1, CEBPA. |
| | | | | | | KIT (c.2446G > A and |
| | | | | | | c.2447A > T) D816I |
| | | | | | | (GAC > ATC)DETECTED |
| | | | | | | The presence of the double |
| | | | | | | mutation at c.2446 and c.2447 |
| | | | | | | in KIT was confirmed by |
| | | | | | | sequencing. |
| AML14 | 16-0120 | 16-05-013 | CK | 48.5 | 43~46, XY, add(2)(q31), add(3)(p21), | TP53 p.Leul14Cysfs*9 (44.9%) |
| | | | with | | add(4)(q21), | and p.? (40.6%) |
| | | | monosomy | | del(5)(q15q33), −7, +8, −12, | |
| | | | | | add(13)(q34), | |
| | | | | | add(15)(p11.2), −16, −18, +1~2mar[cp20] | |
| AML15 | 20-0152 | 20-01-008F | CK | 88.5 | 46~49, XY, +8, t(8; 16)(p11.2; p13.3), | TET2 (c.5081T > A; p.Leul694*; |
| | | | without | | add(22)(p 11.2), +mar[cp18]/46, | 41%) |
| | | | monosomy | | XY[2] | |
| | | | | | | TET2 (c.1936dup; |
| | | | | | | p.Thr646Asnfs*35; 43%) |
| | | | | | | VUS in ASXL1 (c.3538G > C; |
| | | | | | | p.Asp1180His; 49%) |
| AML16 | AAA251 | 120224B | Monocytic | 89 | 46, XX[20] | NGS not done. |
| | | | leukemia | | | Negative for mutations in FLT3, |
| | | | | | | NPM1, CEBPA, cKIT |
| AML17 | 14-0036 | 14-04-020 | CK | 70 | 40-42, XX, add(3)(q12), | Not performed |
| | | | with | | add(5)(q11.2), −7, −9, −12, | |
| | | | monosomy | | add(16)(q12), −17, | |
| | | | | | add(18)(q21), 19, −20, −22, +1-2mar[cp20] | |
| AML18 | 20-0166 | 20-12-013F | TP53 | 81 | 46, XY, t(11; 19)(q23; p13.1)[9]/47, | TP53 p.Arg248Trp (45%) |
| | | | mutated | | idem, +mar[11] | |
| | | | | | | Tier II: NRAS p.Gly12Cys |
| | | | | | | (88%) |
| AML19 | 14-0053 | 14-06-002 | NK | 70 | 46, XX[20] | NGS not done. |
| | | | | | | IDH2 mutation detected. No |
| | | | | | | mutations detected in FLT3, |
| | | | | | | NPM1, CEBPS |
| AML20 | 17-0095 | 17-05-007 | NK | 79 | 46, XX[20] | NPM1 p.Trp288Cysfs*12 |
| | | | | | | (38.6%) |
| | | | | | | BCOR p.Cys1606Asnfs*11 |
| | | | | | | (40.2%) |
| | | | | | | NRAS p.Gly13Asp (VAF |
| | | | | | | 43.2%) |
| AML21 | 17-0219 | 17-09-045 | TP53 | 90 | 42~46, XY, −Y, +8, | TP53 p.Cys275Tyr (77%) and |
| | | | mutated | | der(9)t(9; 11)(q34; q12), | TP53 p.Thr102Profs*21 (11%) |
| | | | | | der(12; 17)(q10; q10), | |
| | | | | | der(17)t(3; 17)(q13; p12)[cp20] | |
| AML22 | 18-0061 | 18-04-004 | TP53 | 71.5 | 45~47, XX, −5, | TP53 p.Phe113Val (45%) |
| | | | mutated | | add(7)(q11.2), +8, +11, | |
| | | | | | der(11)t(11; 11)(p15; q13), add(16)(q21), | |
| | | | | | psu did(17; 5)(p11.2; | |
| | | | | | q22), −17, −20, +1~2mar[cp16]/46, | |
| | | | | | XX[4] | |
| AML23 | AAA320 | 130403A | Monocytic | 48.5 | 46, XY[20] | NGS not done. |
| | | | leukemia | | | Negative for cKIT. |
| AML24 | AAA088 | 101116A | Monocytic | 40 | 46, XX[20] | NGS not done. |
| | | | leukemia | | | |
| AML25 | AAA255 | 120306A | Monocytic | 54.3 | 45, XY, inv(3)(q21q26.2), −7[17]/45, | NGS not done. |
| | | | leukemia | | idem, del(5)(q22q35)[3] | |
| | | | | | | Positive for cKIT mutation. |
| | | | | | | Negative for mutations in FLT3, |
| | | | | | | NPM1, CEBPA, BCR/ABL1 |

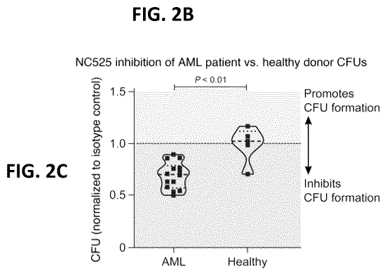

Colony formation of AML bone marrow cells identifies leukemic cells that are broadly defined as progenitors of leukemic blasts, and thus colony formation can be used to quantify LSCs (Sutherland et al. 2001). AML patient bone-marrow cells were cultured with titrated concentrations of LAIR-1 mAb to facilitate increasing levels of LAIR-1 engagement. Compared to isotype or vehicle controls, LAIR-1 by LAIR-1 mAb engagement significantly decreased CFU formation in bone-marrow from AML patients in a dose-response manner (FIG. 2A, 2B). However, LAIR-1 engagement by LAIR-1 mAb on bone-marrow cells from healthy control donors elicited no change in CFU formation (FIG. 2C), suggesting that LAIR-1 uniquely regulates atypical self-renewal in LSCs.

### LAIR-1 Engagement Eradicates Primary and Secondary AML in Patient Derived Xenograft Models

To test the in vivo impact of LAIR-1 on LSCs, AML patient-derived xenograft (PDX) modeling. LSCs from AML patients were engrafted into non-lethally irradiated neonatal mice and human cell proliferation was subsequently measured by quantifying the percent of circulating leukemic cells (FIG. 3A). PDX mice treated with LAIR-1 agonist mAb did not develop disease, with <10% human CD45 CD33 cells in circulation at any time point, while control mice had up to 70% leukemic cells in circulation by 12 weeks post-engraftment (FIG. 3B). LAIR-1-mediated AML suppression was observed across multiple donors and AML subtypes, including normal karyotype AML, monocytic AML, acute myelomonocytic leukemia (AMML), and FLT3⁺ ITD AML, and uncharacterized AML (FIG. 3B). To delineate if the in vivo suppressive effect was due to removal of circulating blasts or eradication of LSCs in the bone-marrow, secondary transplant experiments were performed from PDX donor mice that had been engrafted with either AMML patient LSCs or normal karyotype LSCs. Mouse bone-marrow harvested from PDX animals that had been treated for 4 weeks with either LAIR-1 mAb or isotype control was transplanted into tumor naïve mice (secondary transplant) (FIG. 3C). No further treatment was given after secondary transplant. Mice that received bone-marrow from isotype control-treated donors developed AML. LAIR-1 mAb developed no disease, indicating that LAIR-1 engagement by LAIR-1 mAb agonist mAb eradicated LSCs within the bone-marrow of the PDX donor animals (FIG. 3C).

### A Collagen Matrix is Vital to LAIR-1 Induced AML, Cell Death

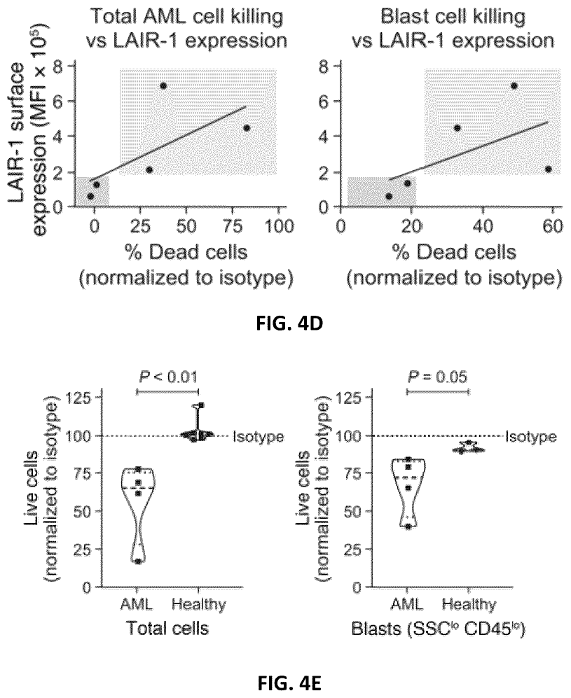

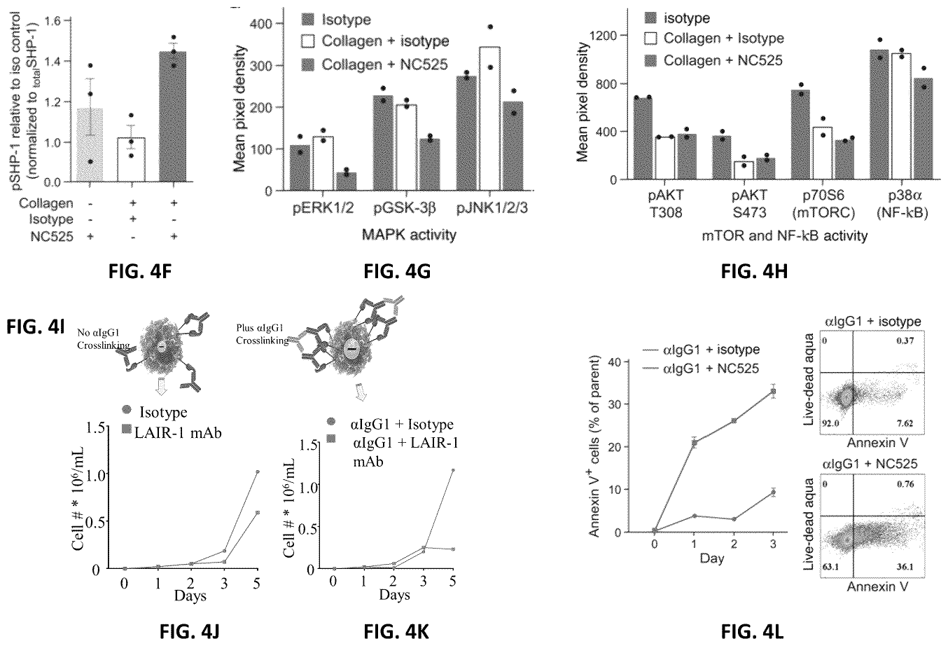

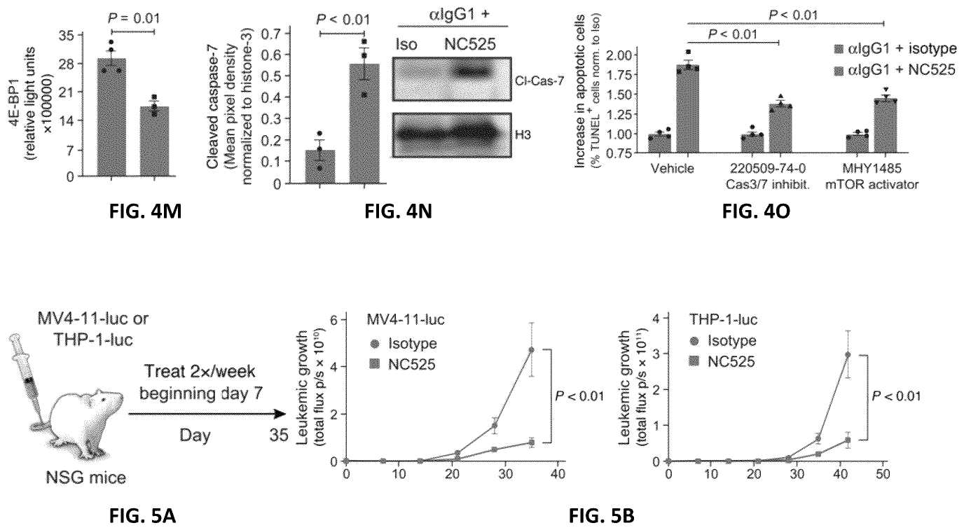

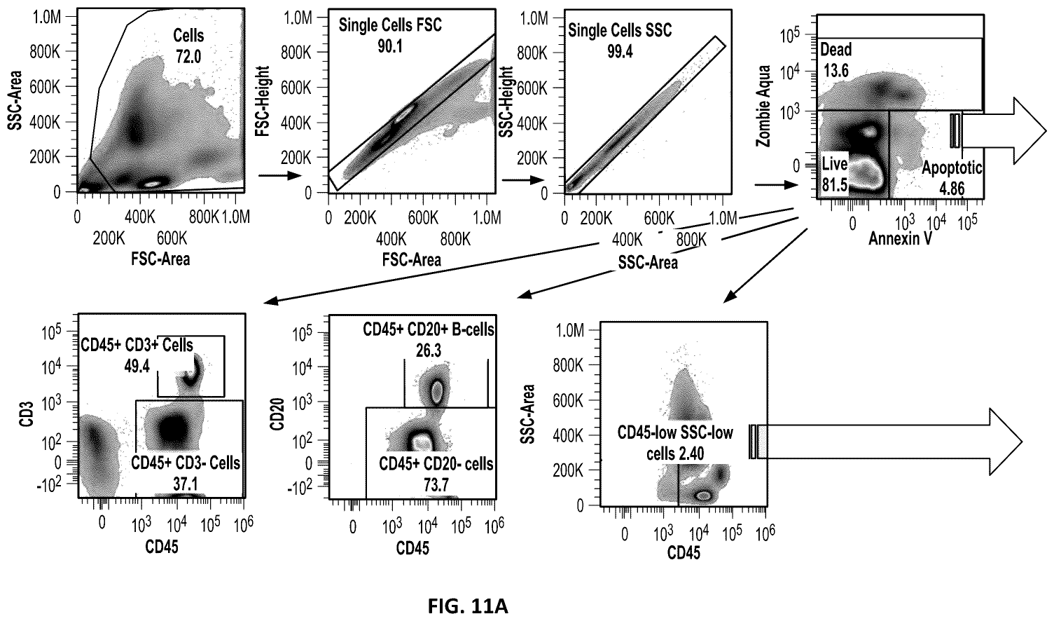

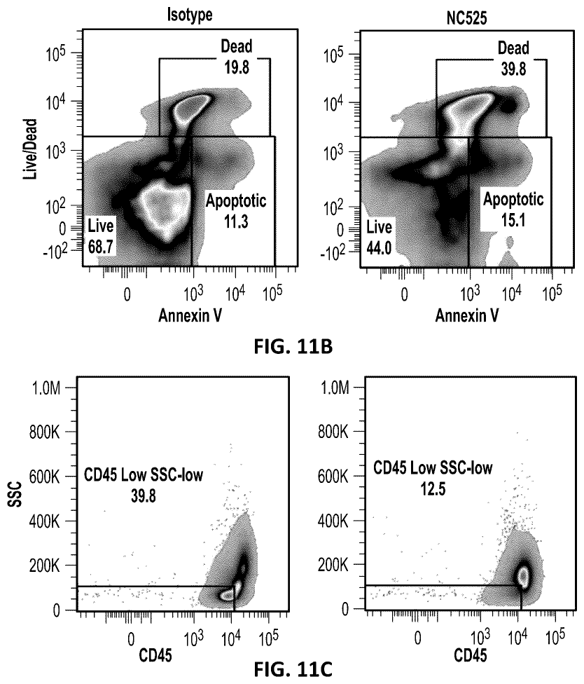

To define the mechanism of LAIR-1-mediated leukemia growth arrest and cell death, AML patient blood samples were tested ex vivo. First, the degree of cell death induced through LAIR-1 ligation was quantified by performing ex vivo culture of red blood cell (RBC)-depleted AML patient whole blood in the presence of LAIR-1 mAb, then measuring live and dead cell populations by flow cytometry (FIG. 11A). In support of the in vivo data, LAIR-1 engagement elicited significant cell death as measured by total AML patient cells (FIG. 11B) or gating on the CD45^{Low} side scatter (SSC) Low blast population (FIG. 11C). Surprisingly, LAIR-1 mediated cell death was dependent on the presence of plate-coated collagen to mimic an extracellular matrix (ECM) (FIG. 4A-C). This was surprising because LAIR-1 agonist mAb blocks collagen binding to LAIR-1 (FIG. 10A). This intriguing finding suggested that leukemic cell fate is dictated by LAIR-1 signal coordination from a collagen matrix. Indeed, some studies have suggested that AML cells can undergo collagen-dependent reprogramming within the bone-marrow niche (Galan-Diez et al, 2018). It was observed that ex vivo leukemic cell death in the presence of collagen was dependent on a threshold of LAIR-1 expression, as patient samples displaying LAIR-1 mean fluorescence intensity above 20000 arbitrary fluorescence units were more receptive to LAIR-1 mAb induced cell death (FIG. 4D). Importantly, LAIR-1 engagement by LAIR-1 mAb did not deplete blood leukocytes from healthy (non-AML) donors, even in the presence of collagen ECM, indicating that LAIR-1 regulation of programmed cell death is specific to AML cell types (FIG. 4E).

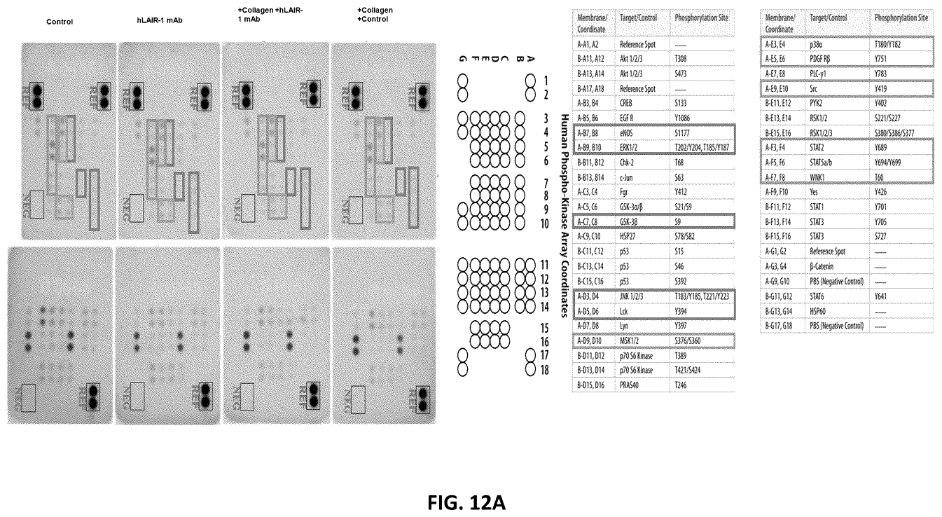

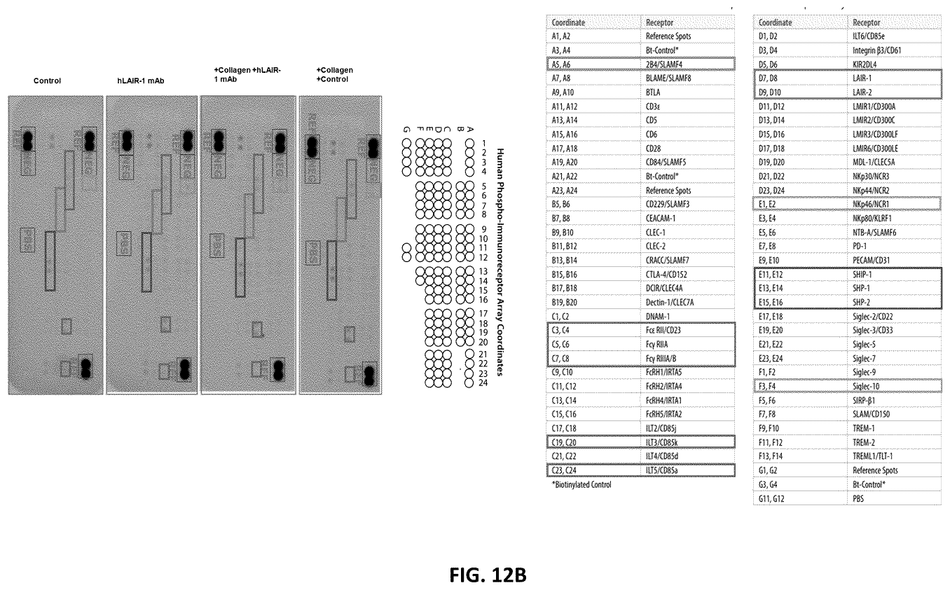

Due to these findings, attempts were made to delineate the phosphorylation network responsible for LAIR-1 mAb-induced cell death in the presence and absence of collagen. Because SHP-1 has been reported as the major adaptor molecule associated with LAIR-1 signal transduction (Zocchi et al., 2001), primary patient AML blasts were first treated with LAIR-1 agonist mAb ex vivo in the presence or absence of collagen and then measured the phosphorylation status of SHP-1. Relative to control, collagen alone elicited a limited increase in pSHP-1, while LAIR-1 mAb increased phosphorylation by 25%, and cotreatment of cells with LAIR-1 mAb and collagen elicited an additive effect, increasing phosphorylation levels by 50% (FIG. 4F). To further extrapolate the signaling axis regulated by LAIR-1 engagement, phospho-immunoreceptors and downstream kinase molecules on PBMCs from a separate AML patient were probed using dot blot arrays. LAIR-1 mAb in conjunction with native collagen increased phosphorylation of multiple ITIM-containing receptors and adaptor molecules, including SHP-1 and LAIR-1 itself (FIG. 12A-12B). Quantification of downstream intracellular phospho-activity during LAIR-1 crosslinking and collagen treatment revealed a pattern of decreased phosphorylation in protein species important for cell growth and survival, including ERK1/2, GSK-3β, and JNK (FIG. 4G and FIG. 12B). Moreover, strong LAIR-1 agonism in the context of collagen decreased activation of major leukemia cell survival factors AKT, mTORC, and NF-kB (FIG. 4H and FIG. 12B). These data illustrate a differential pattern of signaling that occurs with enhanced LAIR-1 signaling in the context of the collagen that is otherwise absent without a collagen matrix.

Based on the data and previous studies suggesting differential LAIR-1 signaling dynamics on AML cells (Poggi et al., 2000; Zocchi et al., 2001), it was hypothesized that a major role of collagen in LAIR-1-mediated cell death is to promote the accumulation of localized LAIR-1 receptors capable of overcoming a signaling threshold that dictates leukemic cell fate. As such, strong LAIR-1 clustering, even in the absence of a collagen matrix would overcome this threshold to result in AML inhibition. To test this, an MV4-11-LAIR-1^{Overexpressing} cell line was utilized. Crosslinking and LAIR-1 clustering on the cell surface were induced by culturing cells with LAIR-1 mAb plus an anti-human IgG antibody that binds to the Fc domain of the agonist LAIR-1 mAb (FIG. 4I). Growth of LAIR-1^{Overexpressing} cells cultured with LAIR-1 mAb alone was only moderately suppressed compared to isotype control (FIG. 4J). However, proliferation of cells cultured under crosslinking conditions was completely inhibited after 3 days of culture (FIG. 4K), with a commensurate increase in annexin V+ cells (FIG. 4L). Because growth was completely arrested by day 5 under crosslinking conditions, it was hypothesized that strong LAIR-1 agonist signaling induced cell death programming. To test this, caspase-3/7 assays were performed on anti-IgG-crosslinked cells at day 5 post-treatment and observed a significant increase in activated caspease-7 activity in the LAIR-1 mAb treatment group (FIG. 4M), indicating that induced clustering and signaling of LAIR-1 by LAIR-1 mAb, as may occur in vivo via Fc receptor enhancement of clustering (Gogesch et al., 2021), can recapitulate collagen-mediated LAIR-1 clustering in vitro to induce a discontinuation signal to leukemic growth processes. We observed significant suppression of the mTORCI target protein 4E-BP1 (FIG. 4M) as well as a substantial increase in activated caspase-7 (FIG. 4N) in the respective NC525 treatment groups. Moreover, NC525-induced apoptosis of AML cells could be partially but significantly reversed by addition of a small-molecule activator of mTOR or a small-molecule inhibitor of caspase-3/7 (FIG. 4O). Meanwhile, NC525 clustering of LAIR-1 in healthy CD34+ cells induced minimal changes in signaling activity. These data further support that NC525 clustering of LAIR-1 induces signaling pathways specific to leukemic cells that inhibit growth processes and promote cell death.

Collectively, these results detail a novel mechanism of control over AML growth, with the LAIR-1 receptor acting as a central collagen response element determining AML fate decisions between growth tolerance or suppression.

LAIR-1 Engagement by LAIR-1 mAb Systemically Reduces AML Growth that is Dependent on LAIR-1 Expression Level, but does not Require or Effect Immune Cells

To evaluate the in vivo dynamics of LAIR-1-mediated suppression of leukemic cell growth, a more tractable cell-derived xenograft (CDX) of AML was used, which allows for mechanistic studies that cannot be readily performed in PDX models. LAIR-1 expression on several AML cell lines was first validated, as shown in Table 3 below.

TABLE 3
Leukemic Cell Lines Tested for LAIR-1 Expression

| Cell | Cell | | FAB | Molecular | |
|---|---|---|---|---|---|
| Line | Type | Morphology | Classification | Mutation | Disease |
| MV4-11 | Macrophage | Lymphoblast | M5 | FLT3 ITD+ | Biphenotypic B |
| | | | | | Myelomonocytic |
| | | | | | Leukemia |
| THP-1 | Monocyte | Monocyte | M5 | NA | Acute monocytic |
| | | | | | leukemia |
| Kasumi | Myeloblast | Myeloblast | M2 | RUNX1- | Acute myeloblastic |
| | | | | RUNX1T1 | leukemia |
| HL-60 | Promyeloblast | Lymphoblast-like | M3 | PML-RARa | Acute |
| | | | | | promyelocytic |
| | | | | | leukemia |
| NB-4 | Myeloblast | Myeloblast | M3 | PML-RARa | Acute |
| | | | | | promyelocytic |
| | | | | | leukemia |
| MOLM-13 | Monocyte | Lymphoblast-like | M5 | FLT3 ITD+ | Acute myeloid |
| | | | | | leukemia |
| MOLM-14 | Monocyte | Lymphoblast-like | M5 | FLT3 ITD+ | Acute myeloid |
| | | | | | leukemia |
| U-937 | Monocyte | Monocyte | M4/5 | NA | Histiocytic |
| | | | | | Lymphoma |
| TF-1 | Erythroblast | Lymphoblast | M6 | NA | Erythroleukemia |
| HEL | Erythroblast | Lymphoblast | M6 | NA | Erythroleukemia |

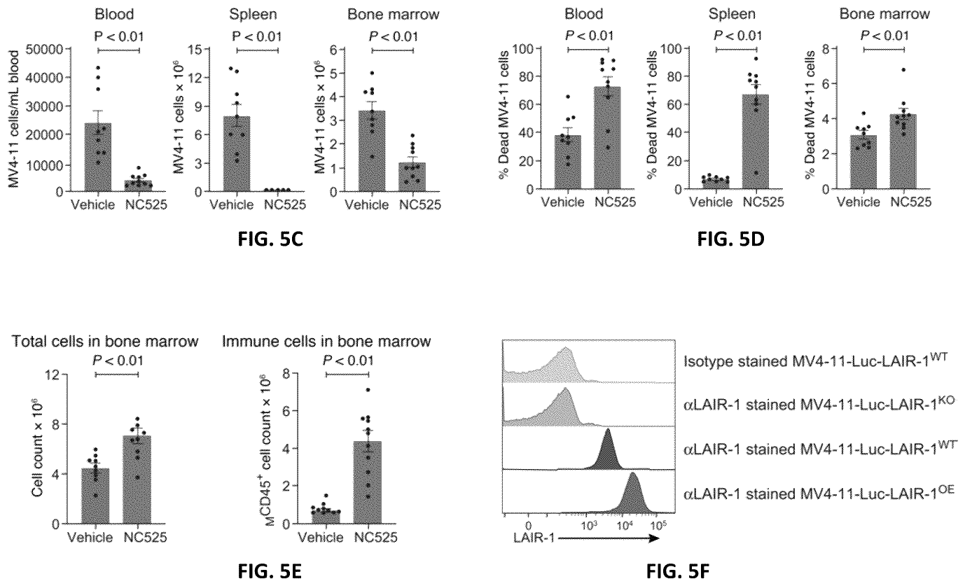

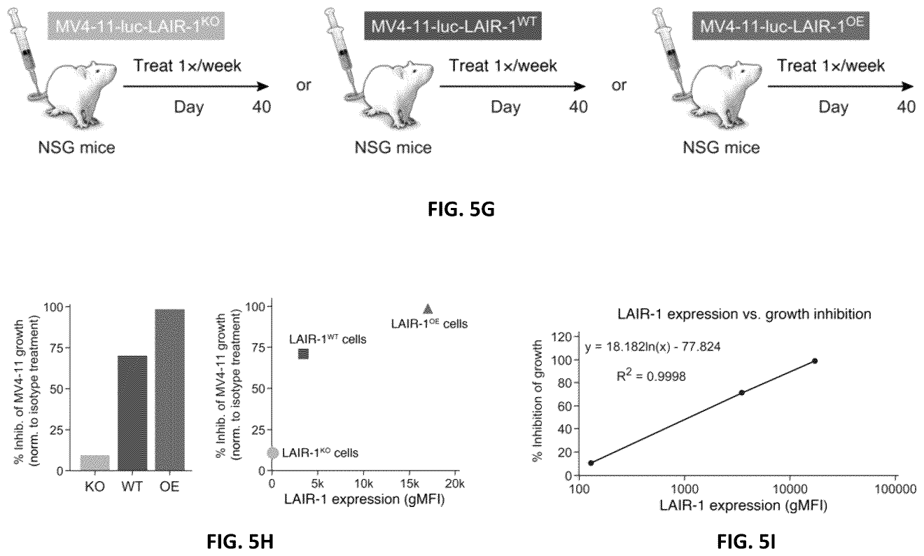

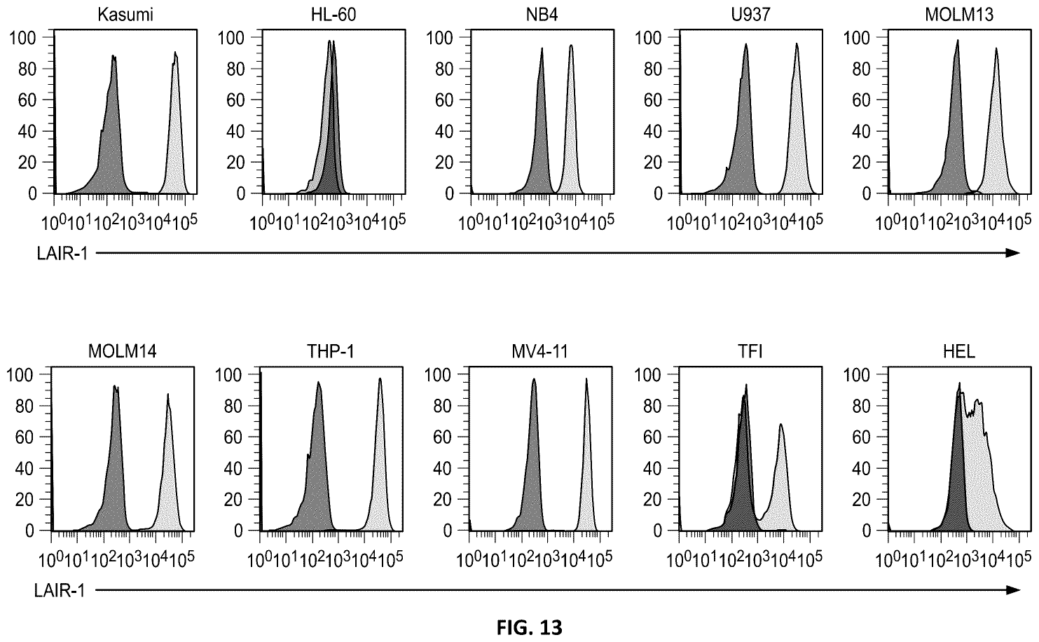

It was shown that most AML cell lines express abundant LAIR-1 (FIG. 13). MV4-11 and THP-1 cells were selected for CDX modeling based on established protocols (Etchin et al., 2013; Cantilena et al., 2022), where naïve NSG mice that lack T or B cells were engrafted with human LAIR1⁺ MV4-11 cells or LAIR-1⁺ THP-1 cells that had been transduced to constitutively express RedFluc luciferase reporter (FIG. 5A). These mice were then treated with LAIR-1 mAb, which engages human LAIR-1 on engrafted leukemic cells but does not engage intrinsically expressed mouse LAIR-1 on murine cells (FIG. 10A-10B). Similar the PDX studies, LAIR-1 engagement by LAIR-1 mAb inhibited leukemic cell growth in vivo in both the MV4-11 CDX model (FIG. 5B-left) and the THP-1 model (FIG. 5B-right).

Focusing on the MV4-11 model, it was found that AML cells were nearly absent in the blood, spleen, and bone marrow of LAIR-1 mAb treated mice (FIG. 5C). Concomitantly, the percentage of dead MV4-11 cells was increased in blood, spleen, and bone-marrow (FIG. 5D), supporting the hypothesis that LAIR-1 engagement actively induced cell death of circulating and tissue-resident AML cells in vivo. Importantly, the inhibition of AML growth in the bone-marrow allowed healthy mouse immune cells to be retained (FIG. 5E). While the MV4-11 CDX model provides a powerful tool to elucidate cell intrinsic effects of LAIR-1 signal transduction on leukemic cells, the model does not extend to potential immune impacting factors because NSG mice do not have an intact human immune compartment.

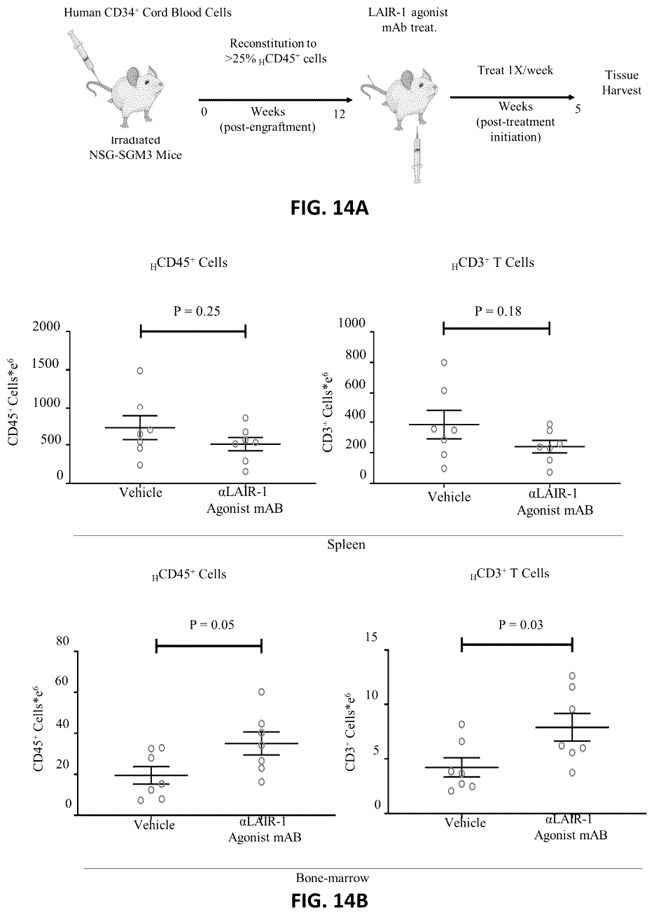

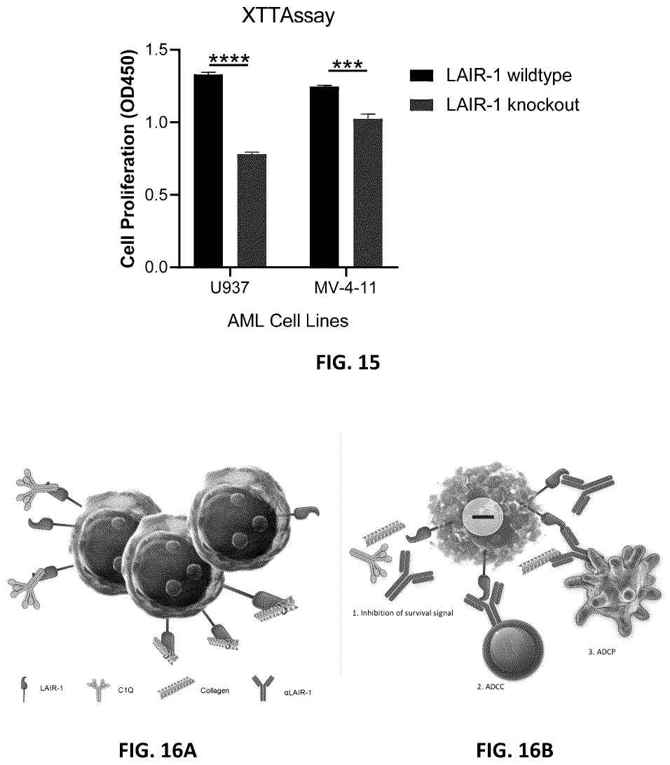

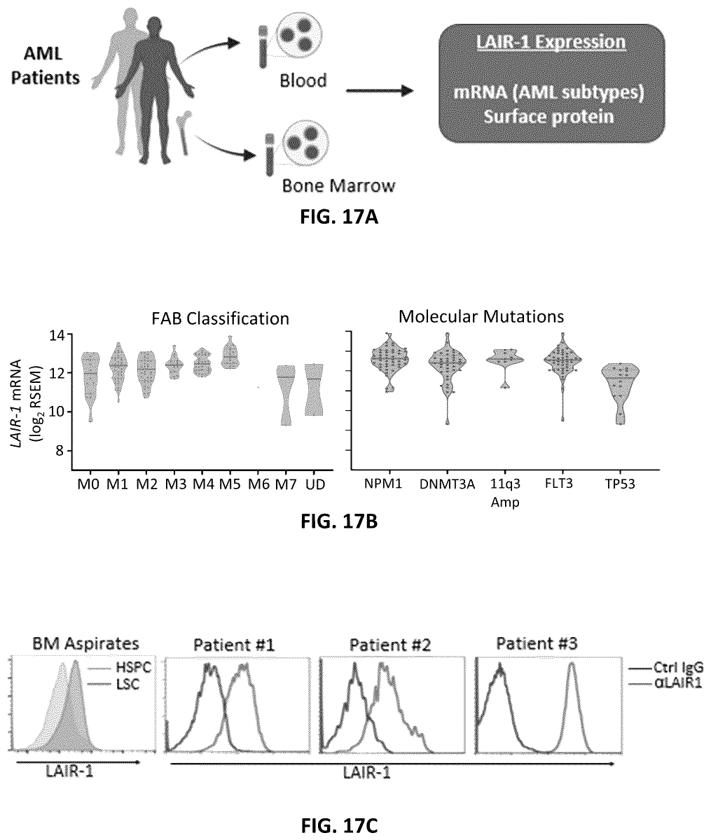

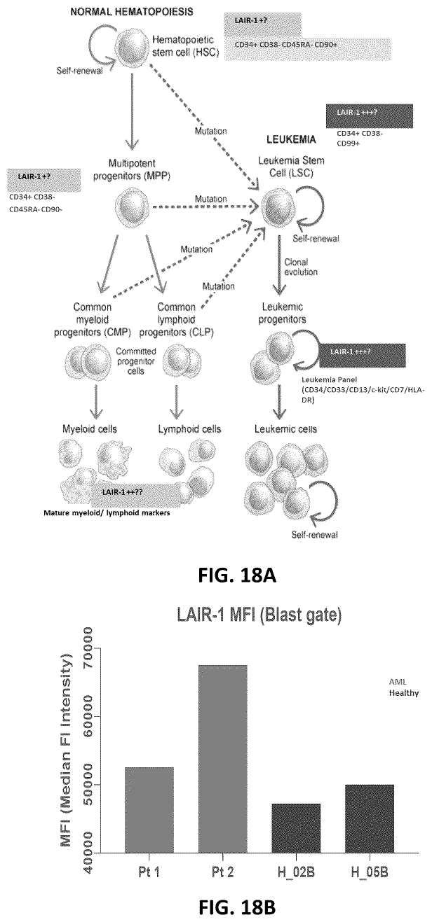

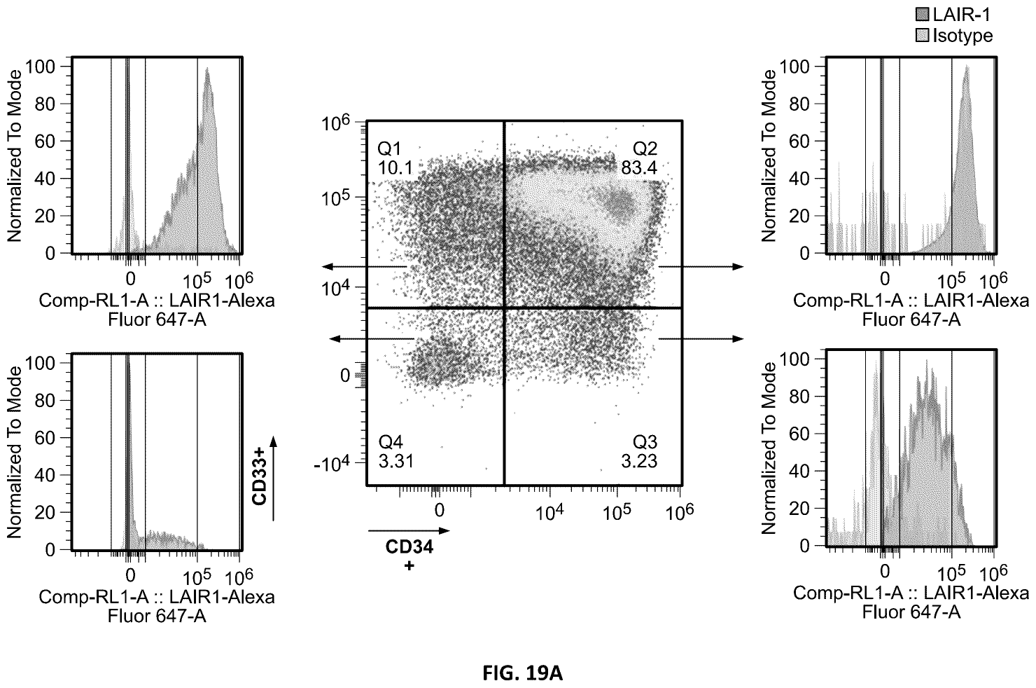

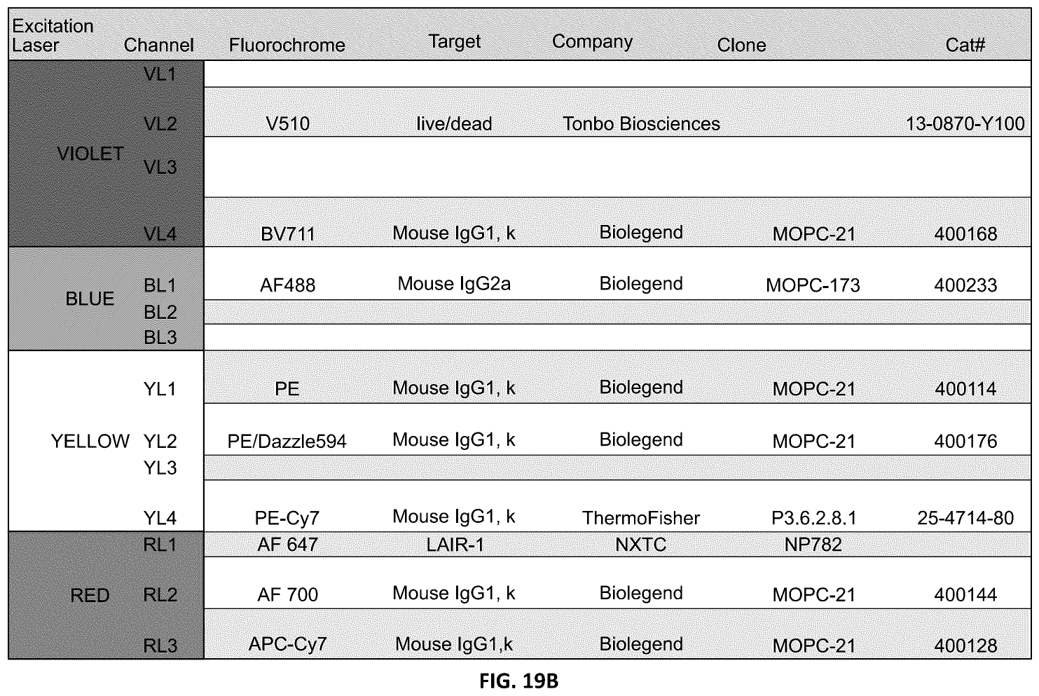

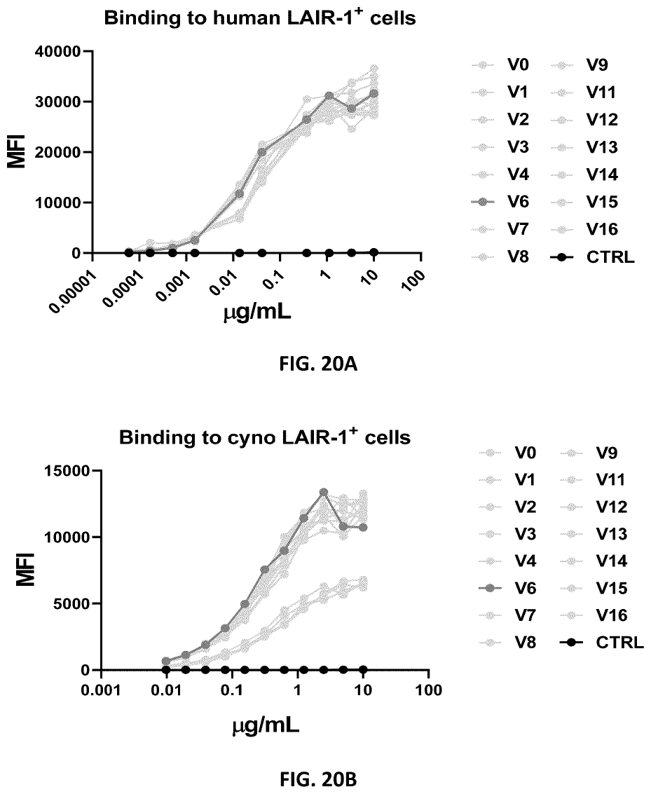

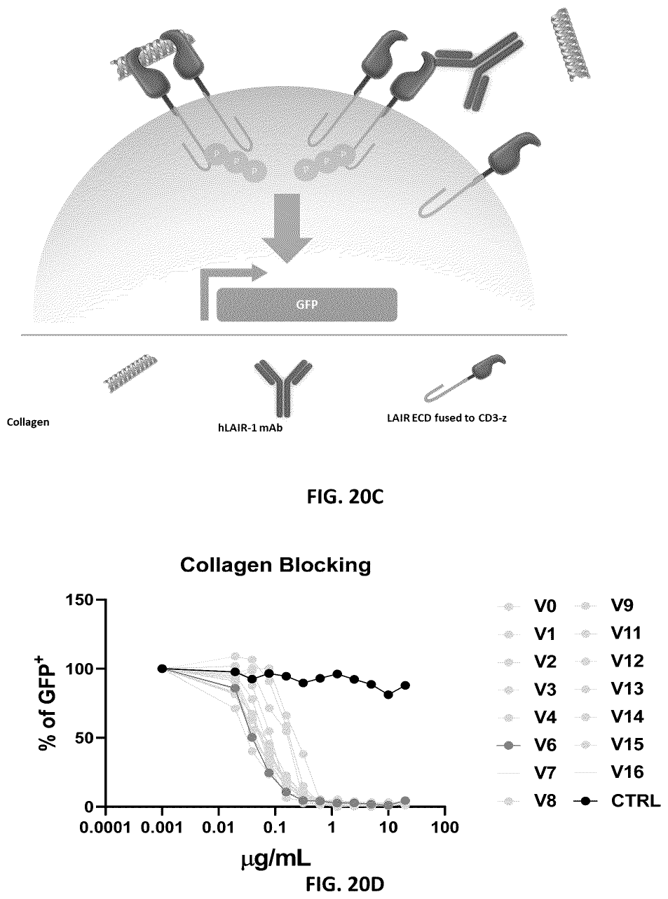

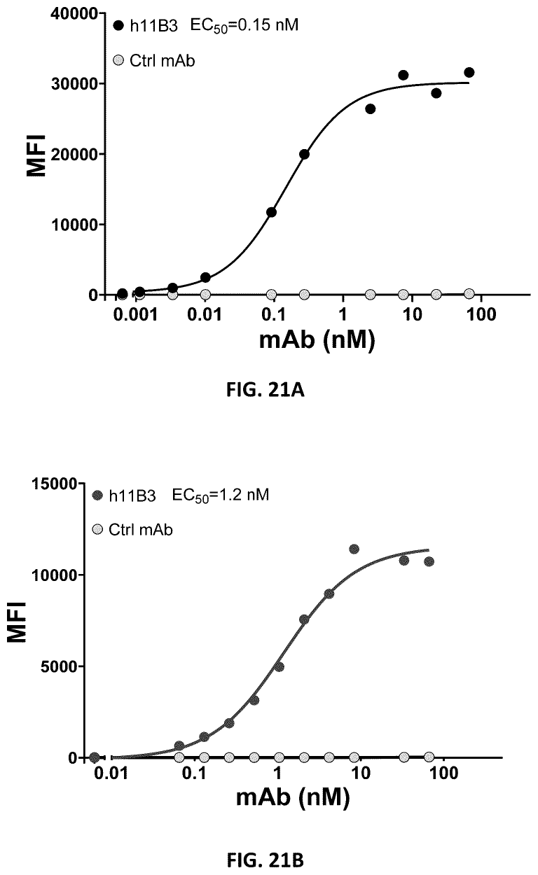

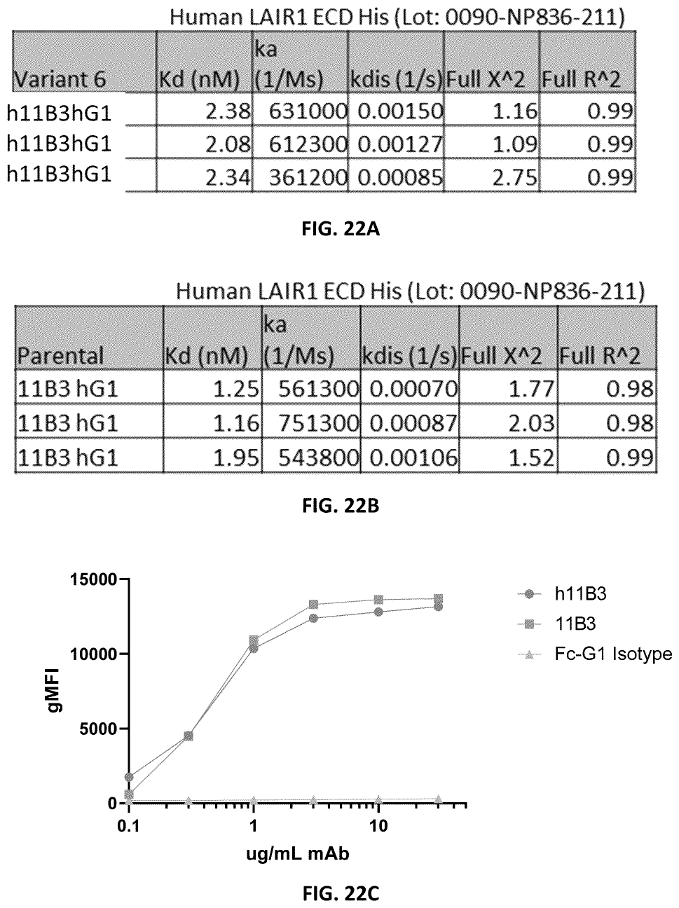

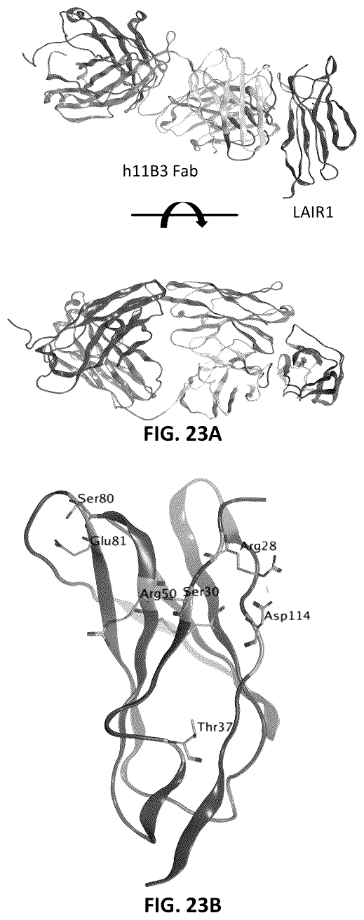

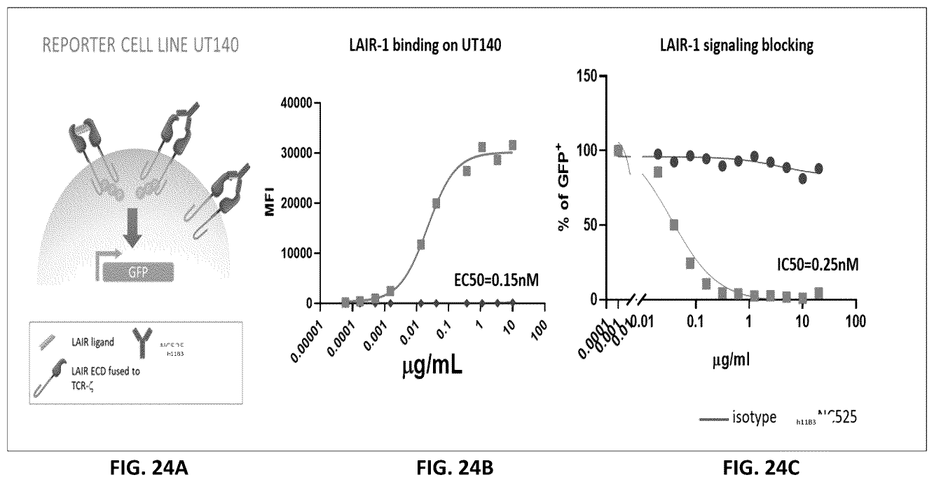

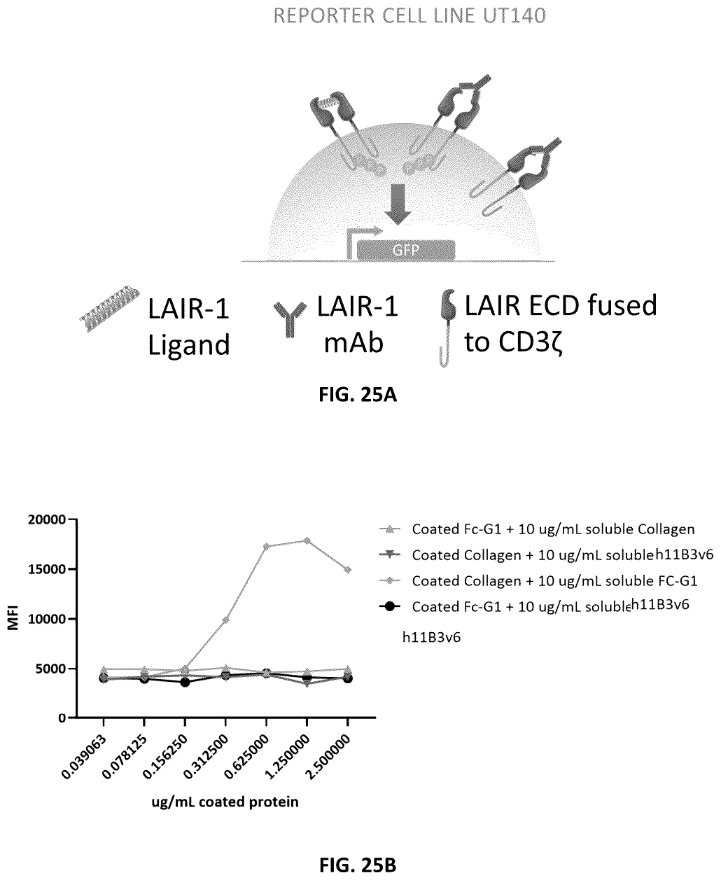

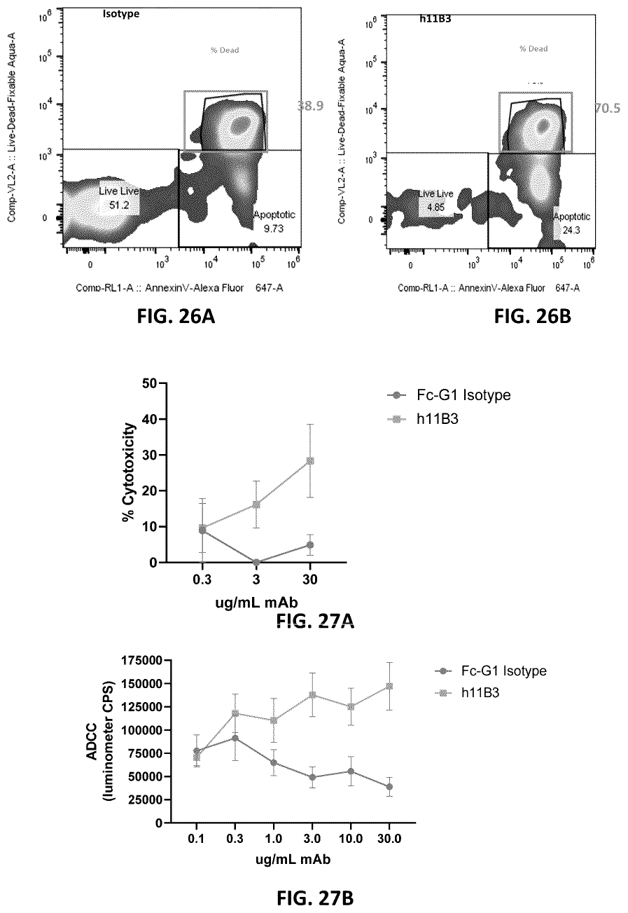

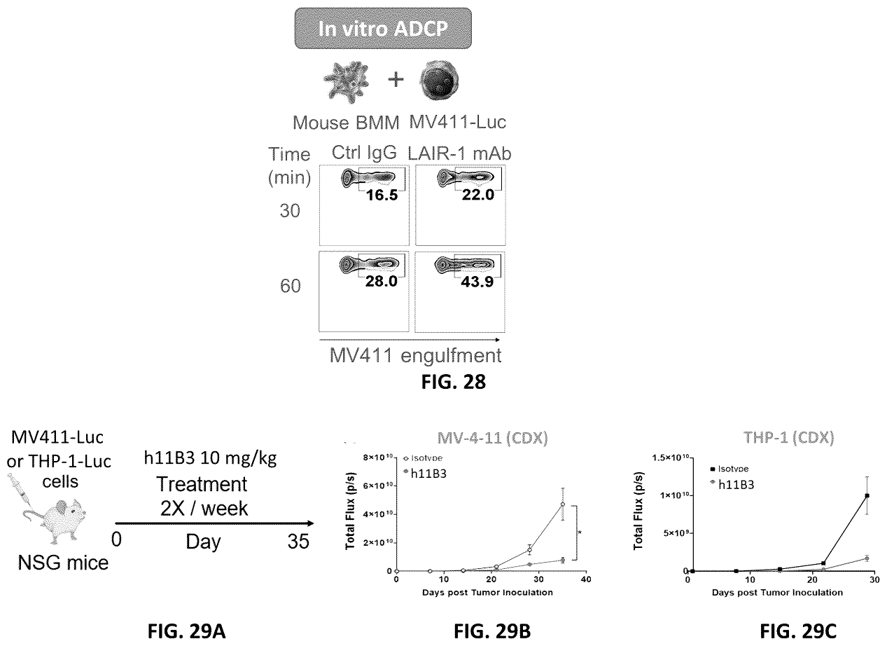

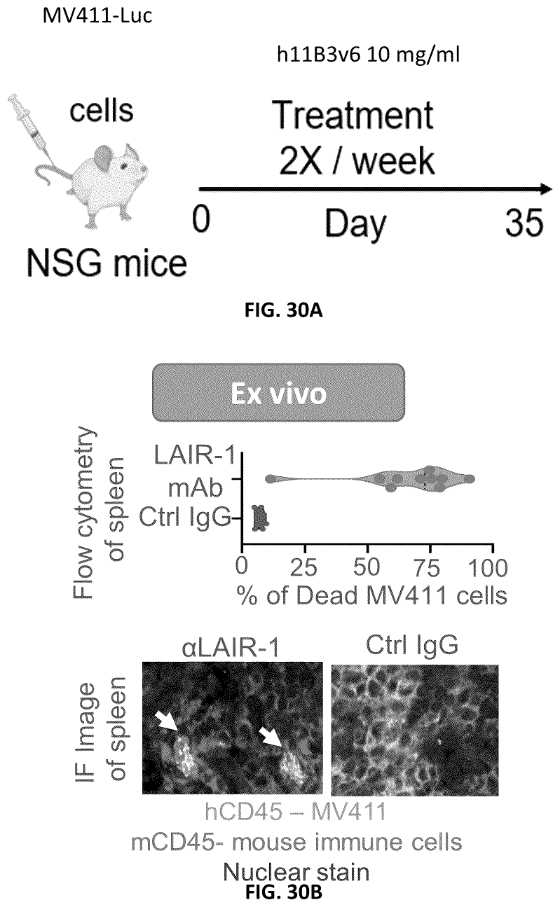

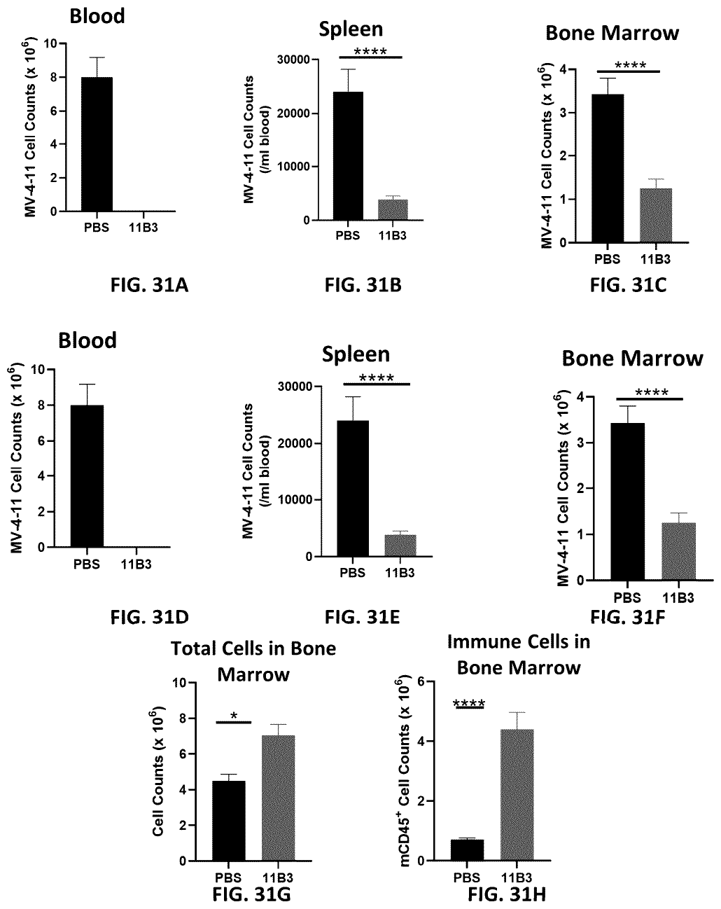

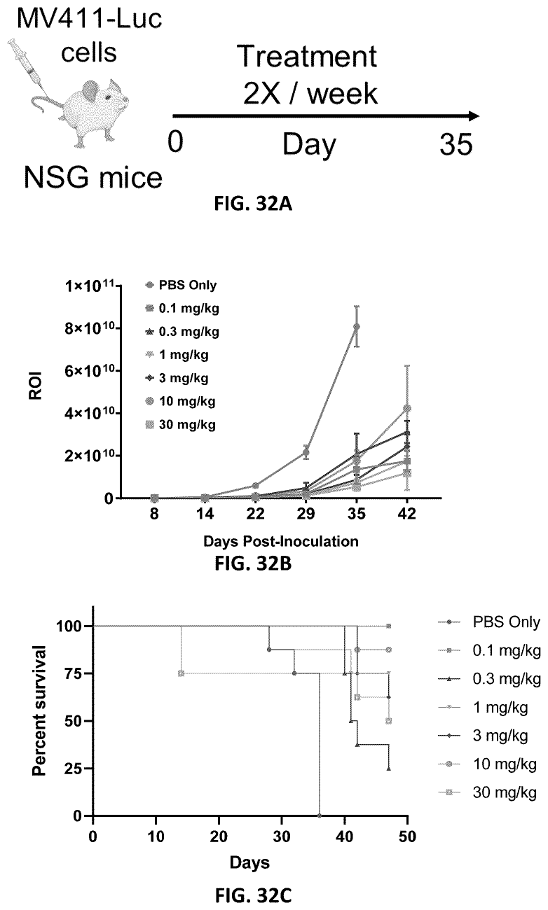

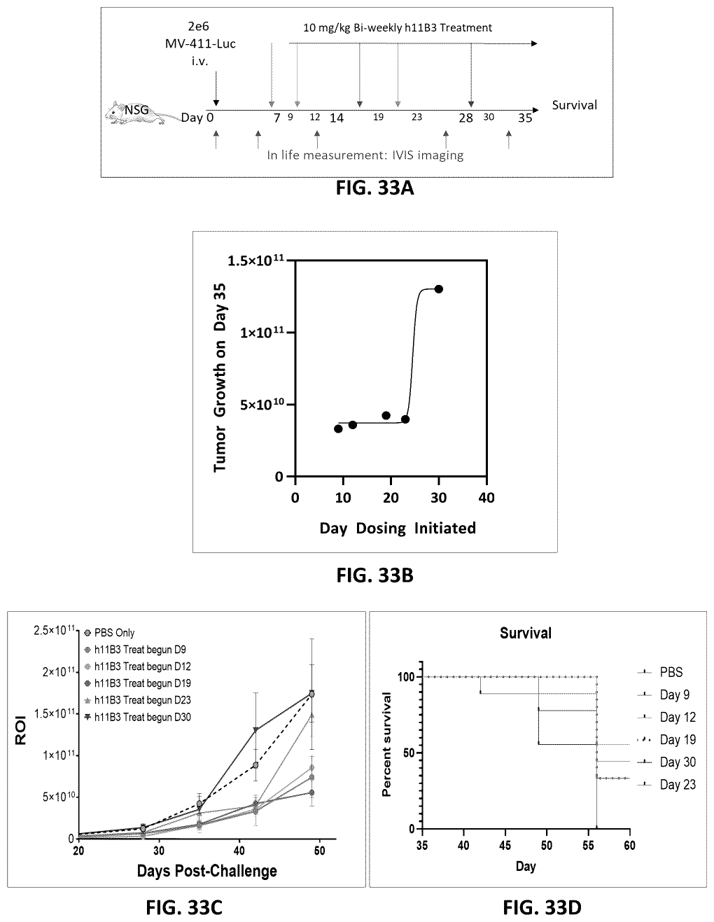

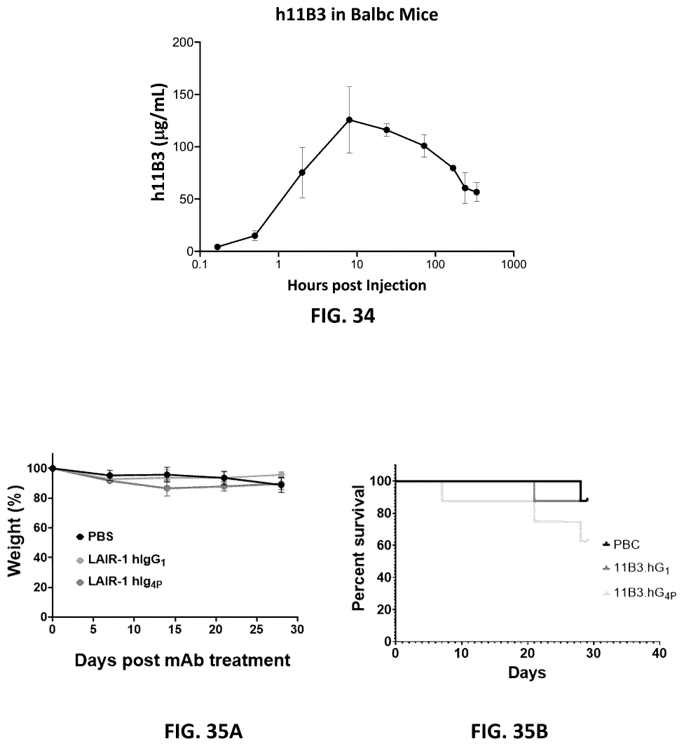

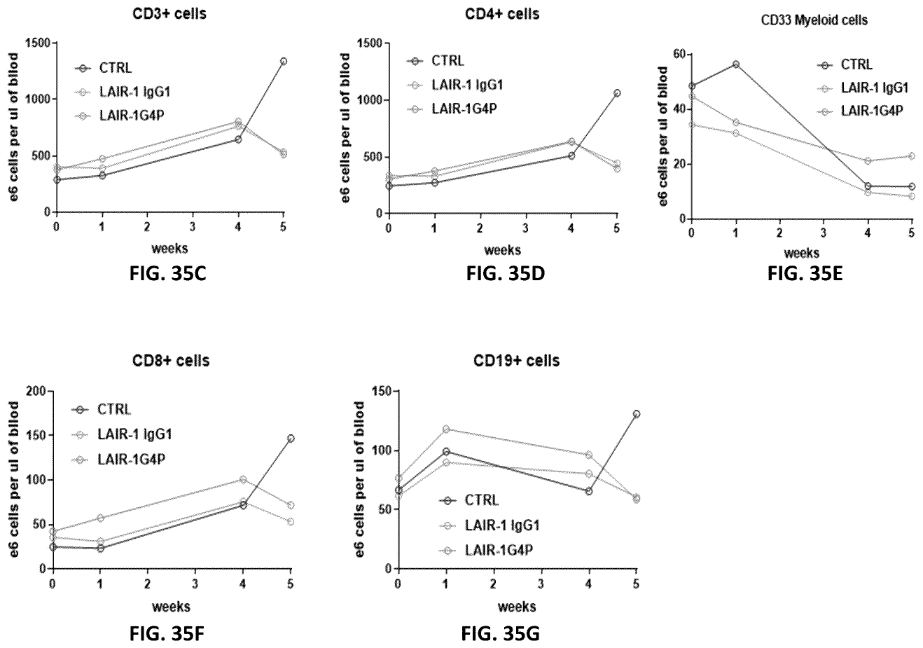

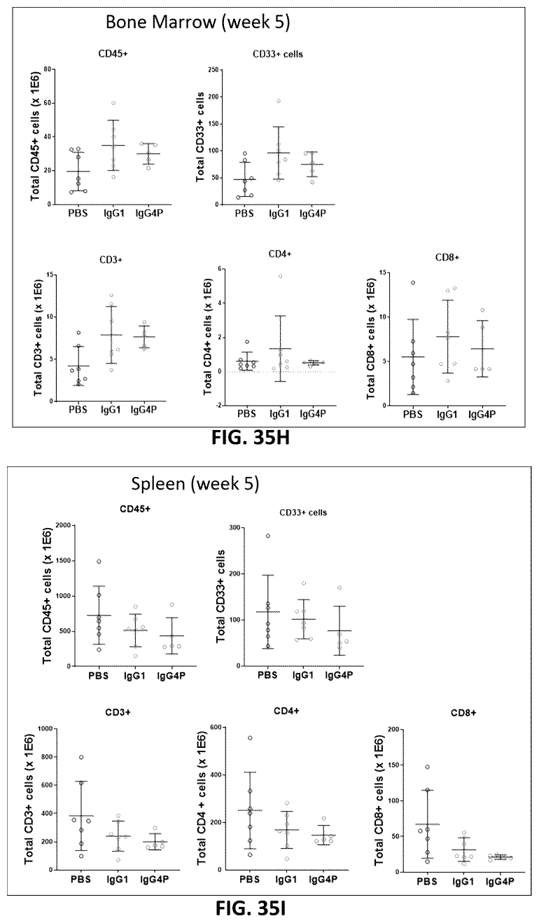

To evaluate the effect of LAIR-1 agonism in the presence of healthy immune cell populations in vivo, LAIR-1 mAb was tested on NSG mice reconstituted for 11-16 weeks with healthy human CD34 stem cells (FIG. 14A). In support of the ex vivo data, this experiment showed that LAIR-1 engagement through LAIR-1 mAb had minimal effects on healthy immune cells in the spleen or the bone-marrow (FIG. 13B).

Because the ex vivo data indicated that a threshold level of LAIR-1 expression was necessary for LAIR-1-induced cell death, it was evaluated whether growth suppression in vivo was also dependent on the level of LAIR-1 surface expression. LAIR-1 knock out (KO) cells were generated in the MV4-11 background—where LAIR-1 is not expressed on the cell surface—and utilized the LAIR-1 overexpressing (OE) cell line—where LAIR-1 is constitutively overexpressed as compared to wild-type (WT) (FIG. 5F). Performing CDX modeling as above (FIG. 5G), it was found that the suppressive effect in vivo was dependent on LAIR-1 surface expression, with LAIR-1 mAb more strongly inhibiting LAIR-1 OE AML growth compared to LAIR-1 WT (FIG. 5H). Inhibition of growth fit a logarithmic curve of LAIR-1 expression y=18−18 ln(x)−77.824, as measured by mean fluorescence intensity (FIG. 5I). These data suggest that arrest of AML cell growth via LAIR-1 signaling is a process regulated through additive signaling dynamics that can be exploited with an agonist mAb such as LAIR-1 mAb.

### LAIR-1 Signaling Restricts AMI, Survival Pathways In Vivo

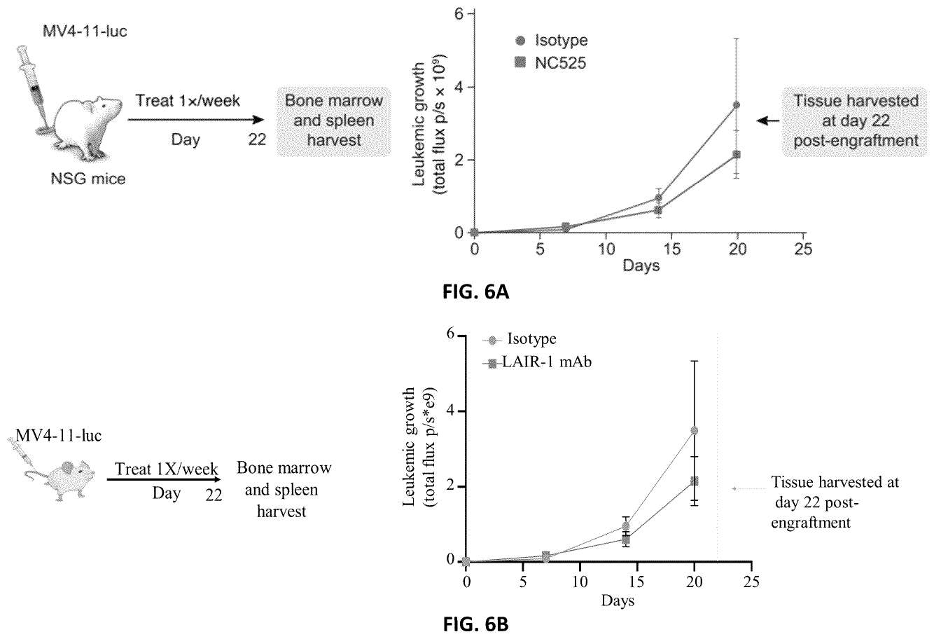

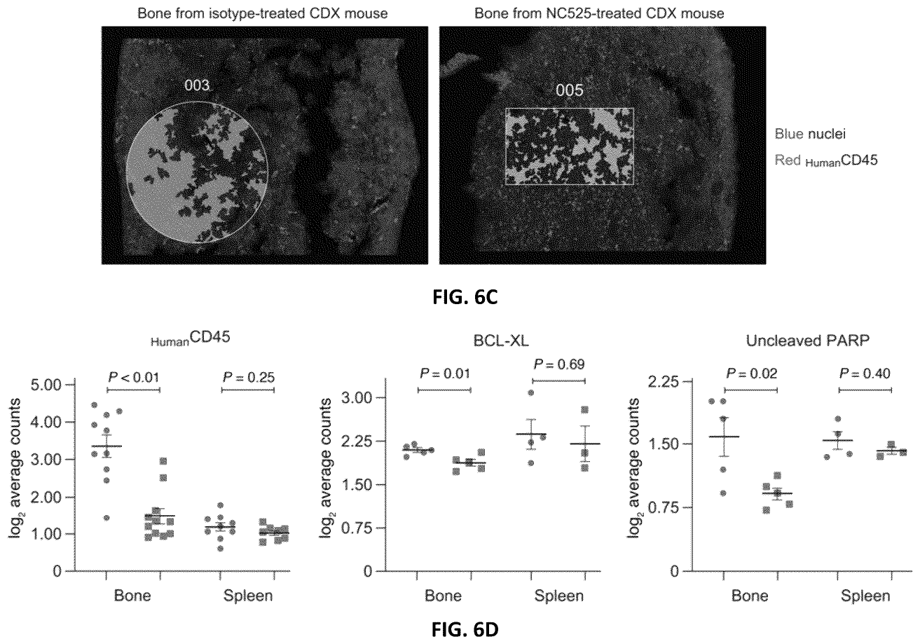

LAIR-1 mAb ligation of LAIR-1 was evaluated in vivo. In order to capture signaling changes at the initiation of leukemic growth suppression, a subcutaneous MV4-11 CDX model was utilized where AML cells could be recovered in sufficient quantity at the timepoint where growth divergence is first observed (FIG. 6A). A phospho-array analysis on in vivo grown MV4-11 cells showed that LAIR-1 mAb significantly suppressed the MAPK pathway and inhibited activation of survival and proliferation molecules mTORC, AKT, and NF-kB in vivo. These data support results with AML patient samples.

While quantifying the phosphorylation status of cell survival molecules is a powerful tool for gaining insight into downstream signaling dynamics, the method is limited to measuring a single posttranslational modification and does not capture non-phosphate mediated signal transduction events which may be important in the leukemic cell fate axis centered on LAIR-1 engagement. To address this, LAIR-1 on leukemic cell homing responses and apoptotic regulation in vivo was evaluated by performing digital spatial imaging on mouse bone-marrow and splenic tissue from LAIR-1 mAb-treated MV4-11 CDX mice. In order to capture temporally dependent cellular changes, murine tissues were harvested at the initiation of MV4-11 growth divergence between treatment groups (FIG. 6A-6B). At this timepoint, LAIR-1 mAb did not elicit any difference in MV4-11 tissue localization (FIG. 6C). However, in support of in vivo CDX disease profiling (FIG. 5B), LAIR-1 engagement did cause a reduction in AML cells (represented by human CD45⁺ cells) in the bones of CDX mice (FIG. 6D). Furthermore, AML cells displayed decreased levels of anti-apoptotic BCL-XL (FIG. 6D) and decreased levels of anti-apoptotic uncleaved PARP (FIG. 6D), though not caspase-9 or BCL6 (data not shown), in bones but not the spleen of mice treated with LAIR-1 agonist mAb. These results indicate that LAIR-1 mAb control of leukemic cell proliferation in vivo likely involves modulation of apoptotic regulators in the collagen-rich bone marrow.

LAIR-1 mAb Synergizes with AMI, Standard-of-Care Therapy

A combination regimen of VEN/AZA, consisting of venetoclax (VEN), which blocks anti-apoptotic B cell lymphoma-2 (Bcl-2) protein, and azacytidine (AZA), which inhibits DNA methyltransferase, has become a standard of care (SoC) for treatment of elderly patient AML. Two of the AML whole-blood samples that responded to ex vivo NC525 treatment were from patients on VEN therapy, and 3 of the samples were from patients previously treated with hypomethylating agents (FIG. 4D and Table 4), supporting the hypothesis that NC525 induces AML cell apoptosis in SoC-treated patient populations. One reason that AML patients become resistant to VEN/AZA is the upregulation of BCL-XL. Because we observed significant reduction in BCL-XL during NC525 treatment, we tested the activity of NC525 with VEN/AZA using ex vivo assays and CDX and PDX in vivo models.

In fresh BM leukemic cells from a patient treated with VEN/AZA, we observed dose-dependent killing of SoC-resistant AML cells by NC525, with up to 70% killing observed at 5 μg/mL of antibody (FIG. 7A). Notably, no impact on healthy T cells or NK cells was observed (FIG. 7B), although BM AML cells had 4-fold higher LAIR-1 expression compared with patient-matched T cells or NK cells (FIG. 7C). Next, the MV4-11 model was used to evaluate NC525 activity in comparison with, and in combination with, AZA or VEN treatments. NC525 monotherapy had significantly better activity than AZA monotherapy at physiologically relevant doses, and AZA cotreatment with NC525 did not inhibit NC525 activity (FIG. 7D). While the physiologically relevant regimen of VEN monotherapy suppressed MV4-11 in vivo growth below the limit of detection within the time frame of the growth curve, survival of mice after challenge was significantly increased in animals treated with the combination of NC525 and VEN over either monotherapy alone (FIG. 7E). To further delineate the potential of NC525 against SoC-resistant AML, PDX modeling using engrafted BM from an additional VEN/AZA-resistant patient was performed. NC525 treatment significantly reduced AML disease as measured by circulating blast cell burden (FIG. 7F) and AML cells in the spleen and BM (FIG. 7G). Combining NC525 with VEN/AZA further reduced AML disease, showing synergistic activity (FIGS. 7F and 7G). These results show not only that LAIR-1 is a viable target for therapeutic intervention of AML, but that the NC525 agonist antibody can work in concert with current clinical therapeutics to eradicate disease and improve patient outcomes.

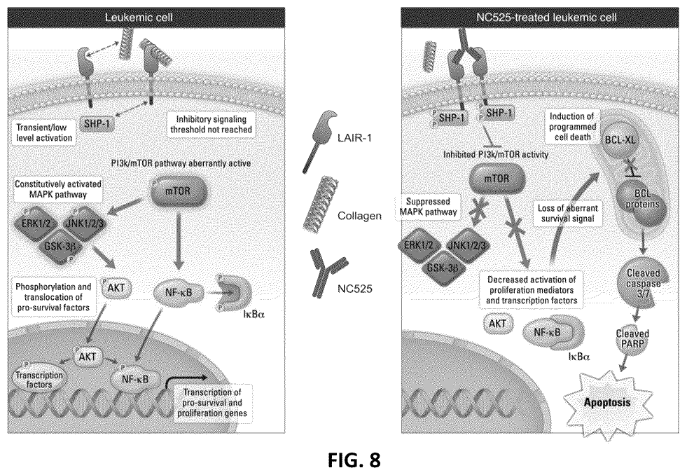

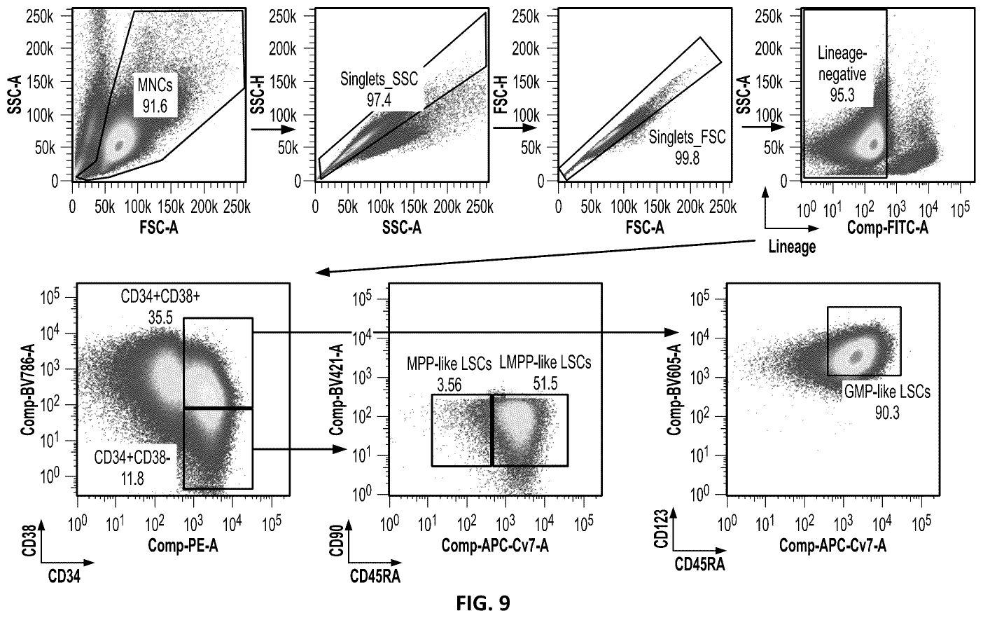

Overall, these collective results indicate that strong LAIR-1 signal induction inhibits growth of leukemic blasts and LSCs, but not immune cells or HSCs, and promotes a programmed cell death phenotype without negatively impacting healthy cells. Importantly, enhanced LAIR-1 signaling in the context of collagen can control AML not only through programmed cell death of blasts, but through LSC depleting. These findings provide critical insight into a novel biological mechanism whereby a collagen matrix may provide survival and maintenance signals to leukemic cells, particularly LSCs in collagen-enriched bone marrow niches. However, enhanced LAIR-1 signaling via an agonist mAb disrupts receptor pathways that are critical to survival, and instead triggers suppressive pathways that downregulate anti-apoptotic stasis, ultimately converging on leukemic cell death (FIG. 8). Agonist targeting of LAIR-1 is thus a unique and promising strategy for AML therapeutic intervention.

TABLE 4
LAIR-1 Expression across AML PDX Models

| | | | | | | | | | FAB |
|---|---|---|---|---|---|---|---|---|---|
| | | | | | | | | | classifi- |
| | | | | Blast II | | CD64+ | | CD117+ | cation/WHO |
| Model | CD45 | CD33 | CD34 | LAIR+ | CD64 | LAIR1+ | CD117 | LAIR1+ | subtype |
| CTG- | + | +/− | + | ++ | weak | ++ | − | ++ | M1 (without |
| 2229 | | | | | | | | | maturation) |
| CTG- | + | + | − | − | ++ | − | − | − | NOS |
| 2232 | | | | | | | | | |
| CTG- | + | +/− | +/− | + | + | + | weak | ++ | AML-MLD; prior |
| 2233 | | | | | | | | | MDS/MPN |
| CTG- | + | + | low/− | − | − | + | − | − | NOS |
| 2234 | | | | | | | | | |
| CTG- | + | +/− | + | − | − | + | ++ | − | AML-MLD; prior |
| 2235 | | | | | | | | | MPN |
| CTG- | + | +/− | + | ++ | ++ | ++ | − | ++ | Biphenotypic |
| 2236 | | | | | | | | | |
| CTG- | + | + | +/− | + | ++ | ++ | weak | + | M4 |
| 2238 | | | | | | | | | (myelomonocytic) |
| CTG- | + | + | − | ++ | ++ | ++ | weak | ++ | AML (11q23 |
| 2240 | | | | | | | | | abnormalities) |
| CTG- | + | weak | + | − | − | − | weak | − | AML-MLD; prior |
| 2453 | | | | | | | | | MDS |
| CTG- | + | + | − | ++ | ++ | ++ | − | ++ | #N/A |
| 2454 | | | | | | | | | |
| CTG- | + | − | + | + | + | + | + | + | NOS |
| 2455 | | | | | | | | | |
| CTG- | + | +/− | + | + | weak | ++ | weak | + | NOS |
| 2456 | | | | | | | | | |
| CTG- | + | − | + | − | − | − | + | − | NOS |
| 2457 | | | | | | | | | |
| CTG- | + | + | − | weak | + | + | − | + | M5 (monocytic; |
| 2701 | | | | | | | | | M5a and M5b) |
| CTG- | + | + | − | + | weak | ++ | − | + | M1 (without |
| 2704 | | | | | | | | | maturation) |
| CTG- | + | + | − | + | ++ | + | − | + | Not available |
| 2774 | | | | | | | | | |
| CTG- | + | − | + | − | − | − | weak | − | AML-MLD; prior |
| 2775 | | | | | | | | | MPN |
| CTG- | + | + | − | + | − | + | weak | ++ | M4 |
| 3438 | | | | | | | | | (myelomonocytic) |
| CTG- | + | + | +/− | ++ | weak | ++ | − | ++ | Not specified |
| 3439 | | | | | | | | | (prior MDS) |
| CTG- | + | + | +/− | + | weak | + | + | + | NOS |
| 3440 | | | | | | | | | |
| CTG- | + | + | − | weak | − | ++ | − | + | Ml (without |
| 3441 | | | | | | | | | maturation) |
| CTG- | + | + | + | weak | weak | + | + | weak | AML with genetic |
| 3659 | | | | | | | | | abnormalities |
| CTG- | + | + | +/− | + | weak | ++ | + | + | AML with genetic |
| 3660 | | | | | | | | | abnormalities |
| CTG- | + | +/− | + | − | − | + | ++ | − | AML-MLD; prior |
| 3661 | | | | | | | | | MPN |
| CTG- | + | +/− | + | − | weak | weak | ++ | weak | AML-MLD; prior |
| 3663 | | | | | | | | | MDS/MPN |
| CTG- | + | low | +/− | weak | − | ++ | + | weak | M0 (minimally |
| 3667 | | | | | | | | | differentiated) |
| CTG- | + | + | + | − | − | weak | weak | weak | M1 (without |
| 3673 | | | | | | | | | maturation) |
| CTG- | + | + | +/− | weak | weak | ++ | weak | + | M1 (without |
| 3674 | | | | | | | | | maturation) |
| CTG- | + | +/− | + | − | weak | weak | weak | − | M0 (minimally |
| 3679 | | | | | | | | | differentiated) |
| CTG- | + | +/− | + | + | − | + | + | + | AML-MLD; prior |
| 3680 | | | | | | | | | MDS/MPN |

| | IDH1/2 | FLT3 | NPM | |
|---|---|---|---|---|
| Model | status | status | status | Diagnosis |
| CTG- | IDH1 | Wild | Wild | Refractory |
| 2229 | mutant | type | type | |
| | (R132C) | | | |
| CTG- | Wild | ITD | Mutant | De novo |
| 2232 | type | mutant | | |
| CTG- | IDH2 | ITD | Wild | Secondary |
| 2233 | mutant | mutant | type | |
| | (R140Q) | | | |
| CTG- | Wild | ITD | Mutant | De novo |
| 2234 | type | mutant | | |
| CTG- | Wild | Wild | Wild | Secondary |
| 2235 | type | type | type | |
| CTG- | Not | ITD | Wild | Not |
| 2236 | available | mutant | type | available |
| CTG- | Wild | ITD | Wild | De novo |
| 2238 | type | mutant | type | |
| CTG- | Wild | Wild | Wild | De novo |
| 2240 | type | type | type | |
| CTG- | Wild | Non-ITD | Mutant | Secondary; |
| 2453 | type | mutant | | refractory |
| | | (V579A) | | |
| CTG- | #N/A | #N/A | #N/A | #N/A |
| 2454 | | | | |
| CTG- | Wild | ITD | Wild | De novo |
| 2455 | type | mutant | type | |
| CTG- | Wild | Wild | Wild | De novo |
| 2456 | type | type | type | |
| CTG- | Wild | Wild | Wild | De novo |
| 2457 | type | type | type | |
| CTG- | Not | ITD | Mutant | De novo |
| 2701 | available | mutant | | |
| CTG- | Not | ITD | Not | De novo |
| 2704 | available | mutant | available | |
| CTG- | Wild | Wild | Not | Recurrent |
| 2774 | type | type | available | |
| CTG- | Not | Not | Not | Secondary |
| 2775 | available | available | available | |
| CTG- | Wild | Wild | Wild | Relapsed |
| 3438 | type | type | type | |
| CTG- | Wild | ITD | Wild | Secondary |
| 3439 | type | mutant | type | |
| CTG- | Wild | ITD | Wild | De novo |
| 3440 | type | mutant | type | |
| CTG- | Wild | ITD | Mutant | De novo |
| 3441 | type | mutant | | |
| CTG- | Wild | Wild | Wild | De novo |
| 3659 | type | type | type | |
| CTG- | Wild | Wild | Wild | De novo |
| 3660 | type | type | type | |
| CTG- | IDH2 | Not | Wild | Secondary |
| 3661 | mutant | available | type | |
| CTG- | Not | Wild | Wild | Secondary |
| 3663 | available | type | type | |
| CTG- | Pending | Pending | Pending | De novo |
| 3667 | | | | |
| CTG- | Not | ITD | Wild | De novo |
| 3673 | available | mutant | type | |
| CTG- | Pending | Non-ITD | Pending | De novo |
| 3674 | | mutant_{(F612delinsYDLKWEFPRENLEF)} | | |
| CTG- | Wild | Non-ITD mutant | Wild | De novo |
| 3679 | type | (T582_E598dup) | type | |
| CTG- | Wild | Wild | Wild | Secondary; |
| 3680 | type | type | type | refractory |

1-92. (canceled)

93. A LAIR-1 antibody comprising:
a) a variable light chain domain having at least 98%, 99% and 100% sequence identity to sequences selected from the group consisting of SEQ ID NOs: 7, 14, 15, 16 and 17; and
b) a variable heavy chain domain having at least 98%, 99% and 100% sequence identity to selected from the group consisting of SEQ ID NOs: 9, 21, 22, 23, 24, 26, 27, 28, 29, 30, 31, 32, 33, 34, 35, 36, 37, 38, 39, 40, 41, 42, 43 and 44.

94. The LAIR-1 antibody of claim 93, wherein the variable light chain domain has at least 98%, 99% and 100% sequence identity to sequences selected from the group consisting of SEQ ID NOs: 14 and 16 and the variable heavy chain domain having at least 98%, 99% and 100% sequence identity to selected from the group consisting of SEQ ID NOs: 21, 24, 27, 30, 33, 36, 39 and 42, or combinations thereof.

95. The LAIR-1 antibody of claim 94, wherein the combinations thereof comprise the variable light chain domain and the variable heavy chain domain having at least 98%, 99% and 100% sequence identity to sequences selected from the group consisting of SEQ ID NOs: 14 and 21, 14 and 24, 14 and 27, 14 and 30, 14 and 33, 14 and 36, 14 and 39, 14 and 42, 16 and 21, 16 and 24, 16 and 27, 16 and 30, 16 and 33, 16 and 36, 16 and 39, and 16 and 42.

96. The LAIR-1 antibody of claim 94, wherein the combinations thereof comprise the variable light chain domain and the variable heavy chain domain having at least 98%, 99% and 100% sequence identity to SEQ ID NOs: 16 and 27.

97. The LAIR-1 antibody of claim 93, wherein the variable light chain domain has at least 98%, 99% and 100% sequence identity to sequences selected from the group consisting of SEQ ID NOs: 15 and 17 and the variable heavy chain domain having at least 98%, 99% and 100% sequence identity to selected from the group consisting of SEQ ID NOs: 22, 23, 25, 26, 28, 29, 31, 32, 34, 35, 37, 38, 40, 41, 43 and 44 or combinations thereof.

98. The LAIR-1 antibody of claim 97, wherein the combinations thereof comprise the variable light chain domain and the variable heavy chain domain having at least 98%, 99% and 100% sequence identity to sequences selected from the group consisting of SEQ ID NOs: 15 and 22, 15 and 23, 15 and 25, 15 and 26, 15 and 28, 15 and 29, 15 and 31, 15 and 32, 15 and 34, 15 and 35, 15 and 37, 15 and 38, 15 and 40, 15 and 41, 15 and 43, 15 and 44, 17 and 22, 17 and 23, 17 and 25, 17 and 26, 17 and 28, 17 and 29, 17 and 31, 17 and 32, 17 and 34, 17 and 35, 17 and 37, 17 and 38, 17 and 40, 17 and 41, 17 and 43, and 17 and 44.

99. The LAIR-1 antibody of claim 93, wherein the variable light chain domain has at least 98%, 99% and 100% sequence identity to SEQ ID NO: 7 and the variable heavy chain domain having at least 98%, 99% and 100% sequence identity to SEQ ID NO: 9.

100. The LAIR-1 antibody of claim 93, wherein the LAIR-1 antibody comprises an antigen binding domain having at least 98%, 99% and 100% sequence identity to SEQ ID NO: 7 or 9.

101. A pharmaceutical composition for treating a subject in need comprising the LAIR-1 antibody of claim 93, wherein the LAIR-1 antibody induces signaling pathways specific to leukemic cells that inhibit growth processes and promote cell death.

102. The pharmaceutical composition of claim 101, further comprising the presence of collagen, wherein the presence of collagen decreases activation of cell survival factors and promotes cell death.

103. The pharmaceutical composition of claim 101 further comprising the combination of the LAIR-1 antibody with drugs used for a standard care of treatment of cancer in the subject in need thereof.

104. The pharmaceutical composition of claim 101, wherein the subject to be treated has carcinoma, squamous cell carcinoma, leukemia, acute lymphocytic leukemia, acute lymphoblastic leukemia, B-cell lymphoma, T-cell lymphoma, Berketts lymphoma, acute myelogenous leukemia, chronic myelogenous leukemia, promyelocytic leukemia, fibrosarcoma, rhabdomyoscarcoma, melanoma, seminoma, tetratocarcinoma, neuroblastoma, glioma, astrocytoma, neuroblastoma, glioma, schwannomas, fibrosarcoma, rhabdomyoscarama, osteosarcoma, xenoderma pegmentosum, keratoactanthoma, seminoma, thyroid follicular cancer, or teratocarcinoma.

105. The pharmaceutical composition of claim 101, wherein the subject to be treated has acute myeloid leukemia.

106. The pharmaceutical composition of claim 101, wherein the LAIR-1 antibody induces the depletion of leukemic stem cells while sparing healthy stem cells and immune cells.

107. The pharmaceutical composition of claim 102, where the cell survival factors are leukemia cell survival factors AKT, mTORC, and NF-kB.

108. The pharmaceutical composition of claim 101, wherein the LAIR-1 antibody alone induces the depletion of acute myeloid leukemia cells that are resistant to acute myeloid leukemia standard of care therapies alone.

109. The pharmaceutical composition of claim 101, wherein the LAIR-1 antibody is combined with drugs used for a standard care of treatment of acute myeloid leukemia, venetoclax and azacytidine, synergistically induces the depletion of acute myeloid leukemia cells.

110. The pharmaceutical composition of claim 109, wherein the LAIR-1 antibody in combination with venetoclax and azacytidine induces the depletion of acute myeloid leukemia cells that are resistant to acute myeloid leukemia standard of care therapies alone.

111. A method of treating a subject having acute myeloid leukemia comprising administering a pharmaceutical composition comprising a LAIR-1 antibody in combination with venetoclax and azacytidine, wherein administering the pharmaceutical composition synergistically induces the depletion of acute myeloid leukemia cells in the subject.

112. The method of claim 105, wherein the pharmaceutical composition is administered parenterally, orally, or topically.
